(19) United States
(12) Patent Application Publication
(10) Pub. No.: US 2026/0263417 A1
(43) Pub. Date: Sep. 10, 2026

(54) STRUCTURE-BASED OPTIMIZATION OF S6K2 INHIBITOR CD02 TO TARGET MELANOMA

(71) Applicants: THE WISTAR INSTITUTE OF ANATOMY AND BIOLOGY, Philadelphia, PA (US); THE TRUSTEES OF THE UNIVERSITY OF PENNSYLVANIA, Philadelphia, PA (US)

(72) Inventors: Ronen MARMORSTEIN, Swarthmore, PA (US); Jessie VILLANUEVA, Philadelphia, PA (US); Jeffrey D. WINKLER, Wynnewood, PA (US)

(73) Assignees: THE WISTAR INSTITUTE OF ANATOMY AND BIOLOGY, Philadelphia, PA (US); THE TRUSTEES OF THE UNIVERSITY OF PENNSYLVANIA, Philadelphia, PA (US)

(21) Appl. No.: 19/153,631

(22) PCT Filed: Apr. 29, 2024
(86) PCT No.: PCT/US2024/026845

§ 371 Date: Aug. 5, 2025

Related U.S. Application Data
(60) Provisional application No. 63/462,478, filed on Apr. 27, 2023.
(60) Provisional application No. 63/501,353, filed on May 10, 2023.

(51) Int. Cl. A61K 31/4025; A61K 31/381; A61K 31/7088; A61P 35/00; C07D 327/04; C07D 411/10; C12N 15/113

(52) U.S. Cl. CPC A61K 31/4025; A61K 31/381; A61K 31/7088; A61P 35/00; C07D 327/04; C07D 411/10; C12N 15/1137; C12N 2310/14

(57) ABSTRACT

Disclosed herein are compounds of the formula: (I) as well as analogs thereof, wherein the variables are defined herein. Also provided are pharmaceutical compositions thereof. In some aspects, the compounds and compositions provided herein may be used to treat cancers, such as melanoma. Also provided are methods of administering compounds and compositions provided herein to a patient in need thereof, for example, for the treatment of NRAS^{mut} melanoma.

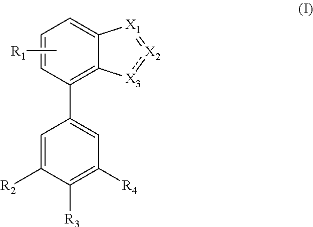

This application claims the benefit of priority to U.S. Provisional Application No. 63/462,478, filed on Apr. 27, 2023, and 63/501,353, filed on May 10, 2023, the entire contents of which are hereby incorporated by reference.

This invention was made with government support under grant CA268510 awarded by the National Institutes of Health. The government has certain rights in the invention.

### BACKGROUND

### I. Field

This disclosure relates to the fields of biology, pharmacology, medicine, and chemistry. In particular, new compounds, compositions, and methods of treatment related to the treatment of melanoma are disclosed.

### II. Description of Related Art

Melanoma is a serious form of skin cancer in humans. It arises from the pigment cells (melanocytes), usually in the skin. The incidence of melanoma is increasing at the fastest rate of all cancers in the United States with a lifetime risk of 1 in 68. Although melanoma accounts for only 4% of all dermatologic cancers, it is responsible for 80% of all deaths from skin cancers.

Although the number of melanoma cancer-related deaths continues to increase and the treatment of advanced melanoma continues to show dismal results, there have been several breakthroughs in the past decade. These breakthroughs include the stratification of melanoma into molecular subtypes, which correlate to prognosis (Viros et al., 2008), as well as targeted therapy, which can be tailored to the specific activated oncogenic pathway (Hodi et al., 2008; and Jiang et al., 2008). For example, specific targeted inhibitors, such as BRAF inhibitors or CKIT inhibitors, such as imatinib mesylate, have been successfully used to treat patients with advanced melanoma (Lutzky et al., 2008). Hence, identifying specific oncogenic pathways in melanoma can help stratify melanoma patients prognostically and can help predict therapeutic results.

A specific subset of melanoma, NRAS^{mut} melanoma, has limited treatment options, and the resulting tumors are extremely aggressive and highly resistant to available target therapies as well as immunotherapies. Oncogenic NRAS activates the MAPK and PI3K pathways, resulting in uncontrolled cellular growth. Inhibiting either pathway alone is insufficient due to feedback activation of the non-inhibited pathway. Cotargeting both pathways, however, leads to toxicities due to the broad range of cellular functions these signaling networks control. Thus, there is a need for small-molecule inhibitors that are able to effectively treat NRAS^{mut} melanoma.

### SUMMARY

In some aspects, the present disclosure provides compounds, pharmaceutical compositions, and methods for their use in the treatment of cancers, such as melanoma.

In some embodiments, the compounds are further defined as:

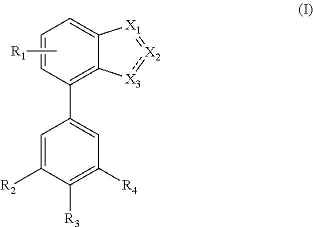

wherein:
   R₁ is hydrogen, hydroxy, halo or amino;
   R₂, R₃, and R₄ are each independently hydrogen, halo, hydroxy, alkylamino_{(C≤8)}, substituted alkylamino_{(C≤8)}, dialkylamino_{(C≤8)}, substituted dialkylamino_{(C≤8)}, or
      —NR_{b}C(X₄)(C(R_{c}R_{d}Rₑ))_{y}, wherein
         R_{b} is hydrogen, alkyl_{(C≤12)}, or substituted alkyl_{(C≤12)};
         R_{c}, R_{d} and Rₑ are each independently absent, halo, hydrogen, hydroxy or amino;
         X₄ is O or NR_{f}, wherein R_{f} is hydrogen, alkyl_{(C≤8)}, or substituted alkyl_{(C≤8)};
         y is 1, 2 or 3; or
      —NR_{b}S(O)₂(C(R_{c}R_{d}Rₑ))_{y}, wherein
         R_{b} is hydrogen, alkyl_{(C≤12)}, or substituted alkyl_{(C≤12)};
         R_{c}, R_{d} and Rₑ are each independently absent, halo, hydrogen, hydroxy or amino;
         y is 1, 2 or 3; or
      —CNR_{b}C(X₄)(C(R_{c}R_{d}Rₑ))_{y}, wherein
         R_{b} is hydrogen, alkyl_{(C≤12)}, or substituted alkyl_{(C≤12)};
         R_{c}, R_{d} and Rₑ are each independently absent, halo, hydrogen, hydroxy or amino;
         X₄ is O or NR_{f}, wherein R_{f} is hydrogen, alkyl_{(C≤8)}, or substituted alkyl_{(C≤8)};
         y is 1, 2 or 3; or
      A group of the formula:

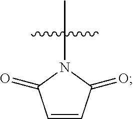 [SMILES: *N1C(=O)C=CC1=O]

provided that R₂, R₃, and R₄ are not each hydrogen; and
X₁, X₂, and X₃ are each independently O, S, C(O), CH, CH₂ or NR_{g}, wherein
   R_{g} is hydrogen, alkyl_{(C≤8)}, or substituted alkyl_{(C≤8)};
or a pharmaceutically acceptable salt thereof.

In some embodiments, the compounds are further defined as:

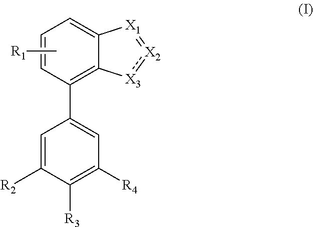

wherein:
   R₁ is hydrogen, hydroxy, halo or amino;
   R₂, R₃, and R₄ are each independently hydrogen, hydroxy, alkylamino_{(C≤8)}, substituted alkylamino_{(C≤8)}, dialkylamino_{(C≤8)}, substituted dialkylamino_{(C≤8)}, or
      —NR_{b}C(X₄)(C(R_{c}R_{d}Rₑ))_{y}, wherein
         R_{b} is hydrogen, alkyl_{(C≤12)}, or substituted alkyl_{(C≤12)};
         R_{c}, R_{d} and Rₑ are each independently absent, halo, hydrogen, hydroxy or amino;
         X₄ is O or NR_{f}, wherein R_{f} is hydrogen, alkyl_{(C≤8)}, or substituted alkyl_{(C≤8)};
         y is 1, 2 or 3; or
      —NR_{b}S(O)₂(C(R_{c}R_{d}Rₑ))_{y}, wherein
         R_{b} is hydrogen, alkyl_{(C≤12)}, or substituted alkyl_{(C≤12)};
         R_{c}, R_{d} and Rₑ are each independently absent, halo, hydrogen, hydroxy or amino;
         y is 1, 2 or 3; or
      —CNR_{b}C(X₄)(C(R_{c}R_{d}Rₑ))_{y}, wherein
         R_{b} is hydrogen, alkyl_{(C≤12)}, or substituted alkyl_{(C≤12)};
         R_{c}, R_{d} and Rₑ are each independently absent, halo, hydrogen, hydroxy or amino;
         X₄ is O or NR_{f}, wherein R_{f} is hydrogen, alkyl_{(C≤8)}, or substituted alkyl_{(C≤8)};
         y is 1, 2 or 3; or
      A group of the formula:

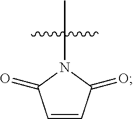 [SMILES: *N1C(=O)C=CC1=O]

provided that R₂, R₃, and R₄ are not each hydrogen; and
X₁, X₂, and X₃ are each independently O, S, C(O), CH, CH₂ or NR_{g}, wherein R_{g} is hydrogen, alkyl_{(C≤8)}, or substituted alkyl_{(C≤8)};
or a pharmaceutically acceptable salt thereof.

In some embodiments, the compounds are further defined as:

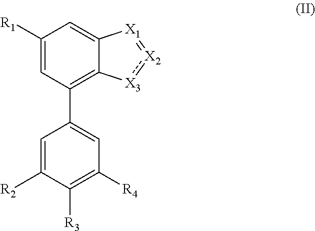

wherein:
   R₁ is hydrogen or hydroxy;
   R₂, R₃, and R₄ are each independently hydrogen, hydroxy, alkylamino_{(C≤8)}, substituted alkylamino_{(C≤8)}, dialkylamino_{(C≤8)}, substituted dialkylamino_{(C≤8)}, or
      —NR_{b}C(X₄)(C(R_{c}R_{d}Rₑ))_{y}, wherein
         R_{b} is hydrogen, alkyl_{(C≤12)}, or substituted alkyl_{(C≤12)};
         R_{c}, R_{d} and Rₑ are each independently absent, halo, hydrogen, hydroxy or amino;
         X₄ is O or NR_{f}, wherein R_{f} is hydrogen, alkyl_{(C≤8)}, or substituted alkyl_{(C≤8)};
         y is 1, 2 or 3; or
      —NR_{b}S(O)₂(C(R_{c}R_{d}Rₑ))_{y}, wherein
         R_{b} is hydrogen, alkyl_{(C≤12)}, or substituted alkyl_{(C≤12)};
         R_{c}, R_{d} and Rₑ are each independently absent, halo, hydrogen, hydroxy or amino;
         y is 1, 2 or 3; or
      —CNR_{b}C(X₄)(C(R_{c}R_{d}Rₑ))_{y}, wherein
         R_{b} is hydrogen, alkyl_{(C≤12)}, or substituted alkyl_{(C≤12)};
         R_{c}, R_{d} and Rₑ are each independently absent, halo, hydrogen, hydroxy or amino;
         X₄ is O or NR_{f}, wherein R_{f} is hydrogen, alkyl_{(C≤8)}, or substituted alkyl_{(C≤8)};
         y is 1, 2 or 3; or
      a group of the formula:

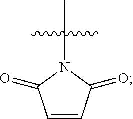 [SMILES: *N1C(=O)C=CC1=O]

provided that R₂, R₃, and R₄ are not each hydrogen; and
X₁, X₂, and X₃ are each independently O, S, C(O), CH or CH₂;
or a pharmaceutically acceptable salt thereof.

In some embodiments, the compounds are further defined as:

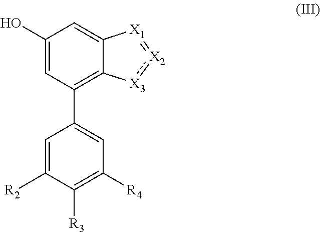

wherein:
   R₂, R₃, and R₄ are each independently hydrogen, hydroxy, alkylamino_{(C≤8)}, substituted alkylamino_{(C≤8)}, dialkylamino_{(C≤8)}, substituted dialkylamino_{(C≤8)}, or
      —NR_{b}C(X₄)(C(R_{c}R_{d}Rₑ))_{y}, wherein
         R_{b} is hydrogen, alkyl_{(C≤12)}, or substituted alkyl_{(C≤12)};
         R_{c}, R_{d} and Rₑ are each independently absent, halo, hydrogen, hydroxy or amino:
         X₄ is O or NR_{f}, wherein R_{f} is hydrogen, alkyl_{(C≤8)}, or substituted alkyl_{(C≤8)};
         y is 1, 2 or 3; or
      —NR_{b}S(O)₂(C(R_{c}R_{d}Rₑ))_{y}, wherein
         R_{b} is hydrogen, alkyl_{(C≤12)}, or substituted alkyl_{(C≤12)};
         R_{c}, R_{d} and Rₑ are each independently absent, halo, hydrogen, hydroxy or amino;
         y is 1, 2 or 3; or
      —CNR_{b}C(X₄)(C(R_{c}R_{d}Rₑ))_{y}, wherein
         R_{b} is hydrogen, alkyl_{(C≤12)}, or substituted alkyl_{(C≤12)};
         R_{c}, R_{d} and Rₑ are each independently absent, halo, hydrogen, hydroxy or amino;
         X₄ is O or NR_{f}, wherein R_{f} is hydrogen, alkyl_{(C≤8)}, or substituted alkyl_{(C≤8)};
         y is 1, 2 or 3; or
      A group of the formula:

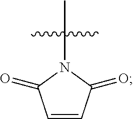 [SMILES: *N1C(=O)C=CC1=O]

provided that R₂, R₃, and R₄ are not each hydrogen; and
X₁, X₂, and X₃ are each independently O, S, C(O), CH or CH₂;
or a pharmaceutically acceptable salt thereof.

In some embodiments, the compounds are further defined as:

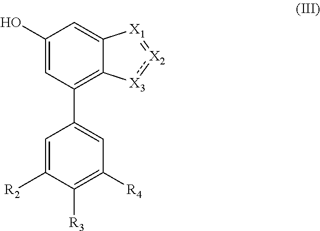

wherein:
   R₂, R₃, and R₄ are each independently hydrogen, hydroxy, alkylamino_{(C≤8)}, substituted alkylamino_{(C≤8)}, dialkylamino_{(C≤8)}, substituted dialkylamino_{(C≤8)}, or
      —NR_{b}C(X₄)(C(R_{c}R_{d}Rₑ))_{y}, wherein
         R_{b} is hydrogen, alkyl_{(C≤12)}, or substituted alkyl_{(C≤12)};
         R_{c}, R_{d} and Rₑ are each independently absent, halo, hydrogen, hydroxy or amino;
         X₄ is O or NR_{f}, wherein R_{f} is hydrogen, alkyl_{(C≤8)}, or substituted alkyl_{(C≤8)};
         y is 1, 2 or 3;
      provided that R₂, R₃, and R₄ are not each hydrogen; and
   X₁, X₂, and X₃ are each independently O, S, C(O), CH or CH₂;
or a pharmaceutically acceptable salt thereof.

In some embodiments, the compounds are further defined as:

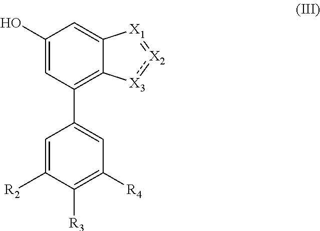

wherein:
   R₂, R₃, and R₄ are each independently hydrogen, hydroxy, alkylamino_{(C≤8)}, substituted alkylamino_{(C≤8)}, dialkylamino_{(C≤8)}, substituted dialkylamino_{(C≤8)}, or
      —NR_{b}S(O)₂(C(R_{c}R_{d}Rₑ))_{y}, wherein
         R_{b} is hydrogen, alkyl_{(C≤12)}, or substituted alkyl_{(C≤12)};
         R_{c}, R_{d} and Rₑ are each independently absent, halo, hydrogen, hydroxy or amino;
         y is 1, 2 or 3;
      provided that R₂, R₃, and R₄ are not each hydrogen; and
   X₁, X₂, and X₃ are each independently O, S, C(O), CH or CH₂;
or a pharmaceutically acceptable salt thereof.

In some embodiments, the compounds are further defined as:

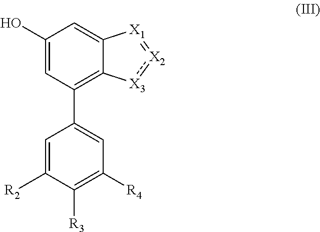

wherein:
   R₂, R₃, and R₄ are each independently hydrogen, hydroxy, alkylamino_{(C≤8)}, substituted alkylamino_{(C≤8)}, dialkylamino_{(C≤8)}, substituted dialkylamino_{(C≤8)}, or
      —NR_{b}C(O)(C(R_{c}R_{d}Rₑ))_{y}, wherein
         R_{b} is hydrogen, alkyl_{(C≤12)}, or substituted alkyl_{(C≤12)};
         R_{c}, R_{d} and Rₑ are each independently absent, halo, hydrogen, hydroxy or amino;
         y is 1, 2 or 3;
   provided that R₂, R₃, and R₄ are not each hydrogen; and
   X₁, X₂, and X₃ are each independently O, S, C(O), CH or CH₂;
or a pharmaceutically acceptable salt thereof.

In some embodiments, R₁ is hydroxy. In some embodiments, R₂ is hydrogen. In other embodiments, R₂ is —NR_{b}C(X₄)(C(R_{c}R_{d}Rₑ))_{y}. In some embodiments, X₄ is O. In some embodiments, R_{b} is hydrogen. In some embodiments, y is 1. In some embodiments, R_{c} and R_{d} are hydrogen. In some embodiments, Rₑ is halo, such as chloro. In some embodiments, R₃ is hydrogen. In other embodiments, R₃ is —NR_{b}C(X₄)(C(R_{c}R_{d}Rₑ))_{y}. In some embodiments, X₄ is O. In some embodiments, R_{b} is hydrogen. In some embodiments, y is 1. In some embodiments, R_{c} and R_{d} are hydrogen. In some embodiments, Rₑ is halo such as chloro. In some embodiments, R₃—N(H)S(O)₂CH₂(Cl). In some embodiments, R₄ is hydrogen. In some embodiments, X₁ is S. In other embodiments, X₁ is O. In yet other embodiments, X₁ is NH. In some embodiments, X₂ is C(O). In other embodiments, X₂ is CH. In some embodiments, X₃ is O. In other embodiments, X₃ is CH.

In some embodiments, the compounds are further defined as:

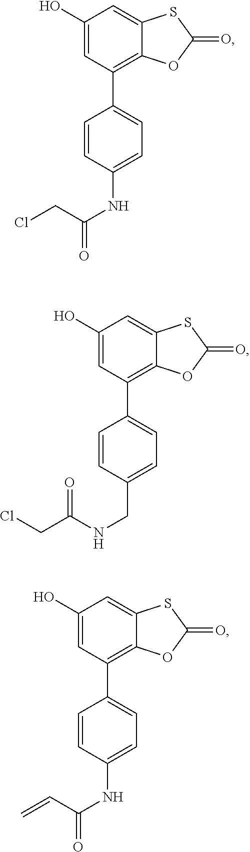 [SMILES: O=C(CCl)Nc1ccc(-c2cc(O)cc3sc(=O)oc23)cc1 ; O=C(CCl)NCc1ccc(-c2cc(O)cc3sc(=O)oc23)cc1 ; C=CC(=O)Nc1ccc(-c2cc(O)cc3sc(=O)oc23)cc1]

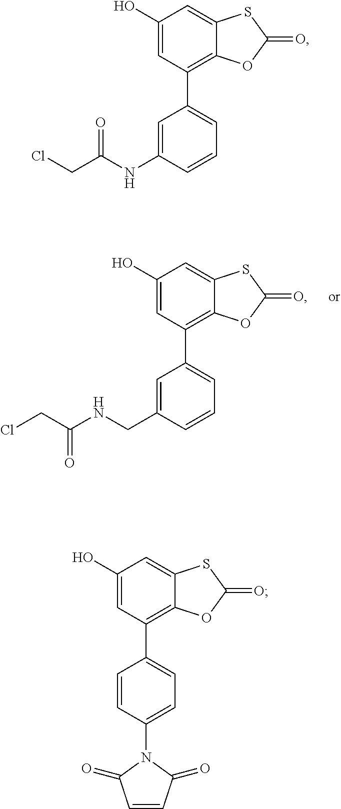 [SMILES: O=C(CCl)Nc1cccc(-c2cc(O)cc3sc(=O)oc23)c1 ; O=C(CCl)NCc1cccc(-c2cc(O)cc3sc(=O)oc23)c1 ; O=C1C=CC(=O)N1c1ccc(-c2cc(O)cc3sc(=O)oc23)cc1]

or a pharmaceutically acceptable salt thereof.

In another aspect, the present disclosure provides pharmaceutical compositions comprising:
(A) a compound described herein; and
(B) a pharmaceutically acceptable excipient.

In some embodiments, the pharmaceutical composition is formulated for administration: orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, parenterally, rectally, subconjunctival, subcutaneously, sublingually, topically, transbuccally, transdermally, vaginally, in cremes, in lipid compositions, via a catheter, via a lavage, via continuous infusion, via infusion, via inhalation, via injection, via local delivery, or via localized perfusion. In some embodiments, the pharmaceutical composition is formulated for oral, intraarterial, or intravenous administration. In some embodiments, the pharmaceutical composition is formulated as a unit dose.

In another aspect, the present disclosure provides a method of treating or preventing a disease or disorder in a patient in need thereof comprising administering to the patient a therapeutically effective amount of a compound or composition described herein. In some embodiments, the disease or disorder is cancer, such as melanoma. In some embodiments, the patient is a mammal, such as a human.

Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description. Note that simply because a particular compound is ascribed to one particular generic formula doesn't mean that it cannot also belong to another generic formula.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1—Inhibitors of MAPK as a single agent elicit limited responses.

FIG. 2—MEK inhibition elicits heterogeneous response in NRAS mutant melanoma.

FIG. 3—S6K1 and S6K2 are highly homologous kinases that have overlapping but distinct biological functions. Both homologs have been implicated in regulating metabolism, including lipid homeostasis and could be good targets for cancer therapy.

FIGS. 4A & 4B—S6K2 expression correlates with poor survival in melanoma. A) S6K2 mRNA levels inversely correlated with S6K1. B) Low expression of S6K2 (not S6K1) associated with survival in patients that received targeted therapies.

FIGS. 5A & 5B—5A Loss of S6K2, but not S6K1 induces cell death in MAPK inhibitor resistant cells. 5B S6K-PPARα subnetwork that is vital for cancer lipid metabolism. Selective inhibition of S6K2 perturbs cellular homeostasis, triggering death of MAPKi-resistant (MAPKi-R) NRAS-mutant melanoma.

FIG. 6—S6K2 inhibitors discovered using ChemBridge Screen.

FIGS. 7A-7C—S6K2i: CD02 induces cell death similar to S6K2 shRNA. A) Loss of S6K2, but not S6K1 by shRNA induces cell death in MAPKi-R cells. B) CD02 induces cell death in MAPKi-R but not MAPKi-S cells. C) RT02 acts as an inactive analog of CD02.

FIGS. 8A & 8B—8A Downstream effects of S6K2i: 1) PPARα upregulation 2) Inhibition of phosphorylation of S6 and 3) Increase in PTGS2 (Marker of ferroptosis) 8B shows the depletion of S6K2 using shRNA.

FIGS. 9A & 9B—S6K2i induces lipid peroxidation similar to S6K2 depletion. A) Induction of Lipid peroxidation (Bodipy C11). B) CD02 induces Lipid Peroxidation in MAPKi-R cell lines.

FIGS. 10A & 10B—S6K2i: CD02, 121 induce ROS similar to S6K2 depletion. A) Induction of ROS. B) CD02 induces ROS in MAPKi-R cell lines.

FIGS. 11A-11C—S6K2i: NAC rescues S6K2i-induced cell death in MAPKi-R cells A) NAC protects M93047 cells from S6K2sh induced cell death. B) Cell death caused by DMSO vs. CD02 and C) GSH, zVAD, and Kira6 attenuate S6K2i-induced cell death with CD02.

FIG. 12—CD02 decreases viability in MAPKi-R 3D Spheroids.

FIG. 13—CD02 induces cell death in MAPKi-R 3D Spheroids.

FIG. 14—S6K2 depletion impairs MAPKi-R NRAS^{mut} 3D Spheroid Formation

FIG. 15—Small molecule library tested for S6K2 activity. A) An initial screen of 44,000 compounds led to the identification of CD02 as an initial hit. B) Modeling of CD02 binding.

FIG. 16—Biological pathway of S6K1/S6K2.

FIG. 17—Alphafold modeling of CD02 docking onto S6K2.

FIG. 18—Mechanism of cysteine interactions with kinase inhibitors.

FIGS. 19A & 19B—Preliminary in vitro studies against MAPKi-R cell lines show more than 10-fold potency for MM-113, in which the chlorine atom of CD02 has been replaced with a chloroacetamide group. Comparative studies show similar inhibition for RT12, a meta analog of MM-113 at 5 μM, but MM-113 shows better potency at lower concentrations. The studies were carried out in M93047 and WM3623 cell lines as shown in FIG. 19B.

FIG. 20—2nd Generation S6K2i, MM-113 is ~10 Fold more potent than CD02.

FIGS. 21A & 21B—The novel S6K2i inhibits viability and induces cell death in acral melanoma. Acral melanoma cells were treated with increasing doses of S6K2i for 72 hours. (A) Viability was assessed by MTT. (B) Cell death measured via Annexin V/PI staining flow cytometry in NRASmut, BRAFmut, and WT/WT acral melanoma cells, respectively.

FIGS. 22A & 22B—Small molecule screen identified nontoxic compounds that synergize with S6K2i. Cells were treated with increasing doses of KDMi+/−S6K2i for 72 hours. Viability was assessed via CellTiterGlo. KDM IC₅₀ was calculated using PRISM. IC₅₀=1.6 μM for the NRAS-mut acral melanoma line WM4235 (A) and an IC₅₀=5 μM for fibroblast (B). Table depicts IC₅₀s for KDMi+/−S6K2i

FIGS. 23A-23F—S6K2i synergizes with KDMi to inhibit viability and induce cell death in acral melanoma. (A, C) S6K2i synergizes with KDMi in both WM4235 (NRASmut) and WM4324 (BRAFmut) acral melanoma cells according to the Bliss independence model. (B, D) S6K2i (12.5 μM)+KDMi (0-5 M) synergistically inhibited cell viability in both WM4235 (NRASmut) and WM4324 (BRAFmut). (E, F) S6K2i and KDMi co-administered synergistically induced cell death as measured via Annexin V/PI staining flow cytometry. Values shown represent n=3±SD. ****, P<0.0001; ***, P<0.001; **, P<0.01; *, P<0.05.

FIGS. 24A & 24B—S6K2i+/−KDMi inhibits acral melanoma colony formation.(A,B) Single agent S6K2i (12.5 and 25 μM) and KDMi (0.25 and 0.5 μM) showed minimal inhibition of WM4235 (A, NRAS^{mut}) and WM4324 (B, BRAFmut); however, S6K2i and KDMi co-administered synergistically inhibited colonies.

FIG. 25—Tumor volume after administration of 50 mg/kg of CD02 using daily ip injections compared to the vehicle administration. N=4

FIGS. 26A-26H—A.-C. NRAS-mutant melanoma cells were treated with MAPK inhibitors to determine dose-response relationships. Cell viability was determined 72 h post-treatment by (A) MTT (trametinib; SCH772984) (mean±SD; n=7) or (B) CellTiter-Glo assays (MEK-162; BVD-523) (mean±SD; n=2). (C) Sensitivity of NRAS-mutant melanoma cells to MAPK inhibitors (A, B) represented as area under the dose-response curve values in a blue-to-red heatmap with white median. D., E. MAPKi-S or MAPKi-R cells were treated with trametinib (100 nM) or SCH772984 (1000 nM) for 72 h. (D) BrdU incorporation relative to DMSO; mean±SD (n=3). The difference in BrdU incorporation between DMSO- and drug-treated cells was calculated for each cell line. MAPKi-S vs. MAPKi-R compared by unpaired, two-tailed Student's t-test. (E) Cell death was assessed by PSVue/PT staining (n=2). Cell death induction was calculated in each cell line and compared in MAPKi-R vs. MAPKi-S cells by unpaired, two-tailed Student's t-test. F. MAPKi-R or MAPKi-S cells were treated with SCH772984 (1000 nM) for 24 h and analyzed by immunoblotting. G. Kinetics of MAPK pathway inhibition. Cells treated trametinib (100 nM) or SCH772984 (1000 nM) were analyzed by immunoblotting. H. Cells were transduced with doxycycline-inducible S6K2^{T388E} and incubated with 0.25 μg/ml doxycycline for 48 h. S6K2 and phospho-S6 (S240/244) levels were determined by immunoblotting i Cells transduced with doxycycline-inducible S6K1T389E were incubated with doxycycline for 24 h and then treated with ERKi SCH984 (1000 nM) or PI3K/mTORi GSK458 (100 nM) for an additional 24 h. Percentage of pS6^{(S240/244)}+ cells was assessed by flow cytometry (n=1).

FIGS. 27A-27D—A. NRAS-mutant melanoma cells were treated for 48 h with the MEKi trametinib (M; 10 nM) or the ERKi SCH772984 (E; 100 nM) and changes in protein abundance or phosphorylation were determined by reverse phase protein array (RPPA) analysis (n=3). Heatmap depicts log 2 fold changes in MAPK, PI3K, mTOR, and S6K pathways relative to vehicle controls. Differences in response between resistant versus sensitive cell lines were calculated with unpaired, two-sided Mann-Whitney tests, indicating differential regulation of S6K signaling between MAPKi-resistant and -sensitive cell lines. B. MAPKi-resistant (M93-047, WM1366) or -sensitive (WM3451, WM4113) melanoma cells were transduced with doxycycline-inducible constitutively active HA-tagged S6K2T388E or empty vector (EV), pre-treated with 0.25 μg/ml doxycycline for 48 h, and then treated with DMSO vehicle (Veh), ERKi SCH984 (1 μM) or PI3K/mTORi GSK2126458 (GSK458, 100 nM) for an additional 24 h. Percentage of pS6(S240/244)+ cells was determined by flow cytometry; bar graphs show mean±SD (n=3); p-values were calculated by unpaired, two-tailed Student's t-tests. C. Cells treated as in (B) were probed by immunoblotting with the indicated antibodies. D. Diagram illustrating that in MAPKi-resistant NRAS-mutant melanoma cells, S6K is primarily regulated by the PI3K/mTOR pathway. In contrast, in MAPKi-sensitive cells, S6K relies on both the MAPK and the PI3K pathways.

FIGS. 28A-28H—A. S6K1 mRNA levels inversely correlate with S6K2 (443 patients; TCGA Skin Cutaneous Melanoma (SKCM); accessed via cBioPortal 5 Feb. 2022). B. Kaplan-Meier survival curve analysis was performed using R₂: Genomics Analysis and Visualization. Overall survival curves are depicted for patients with high vs. low S6K2 or S6K1 expression (TCGA SKCM; n=470; first vs. last quartile cut-off). C. Expression of S6K2 (RPS6KB2) and S6K1 (RPS6KB1) in trametinib-sensitive (MEKi-S; n=12) or -resistant (MEKi-R; n=11) patient-derived NRAS^{mut} melanoma cell lines as determined by RNA sequencing. Three MAPKi-R (D, E) or MAPKi-S (F, G) cell lines were transduced with lentiviruses encoding S6K1 or S6K2 shRNA and analyzed for cell death 7 days post infection (dpi) by Annexin V/PI (D, F) (mean±SD; n=3; unpaired two-tailed Student's t-test) or by immunoblotting (E, G) (n=2). H. M93-047 cells were transduced with constitutively active S6K1^{T389E}, treated with DMSO or the pan-S6K inhibitor LY2584702 (LY4702; 1, 5, 10 μM) for 48 hours and analyzed for cell death (left panel) or by immunoblotting (right panel).

FIGS. 29A-29F—A., B. NRAS-mutant melanoma cells (M93-047) were transduced with lentivirus encoding S6K1 or S6K2 shRNA, or non-targeting vector control (Vec) and subjected to proteomic (A) or lipidomic (B) analysis (4 dpi). A. Heatmap depicting abundance of selected proteins involved in lipid metabolic pathways relative to vector control; p-values denote two-tailed Student's t-tests of shS6K2 vs. shS6K1 adjusted for multiple hypothesis testing at a 10% FDR (Benjamini-Hochberg). B. Box and whiskers plot depicting classes of lipids differentially affected by shS6K2 vs. shS6K1. Median, upper quartile and lower quartiles are shown. Each dot indicates an individual lipid species. Details of lipids are presented in Supplementary Table 4. C-F Three MAPKi-R and three MAPKi-S cell lines were transduced with lentiviruses encoding S6K2 shRNA, S6K1 shRNA or empty vector control (EV). ROS levels (H2DCFDA; C, E) or lipid peroxidation (Bodipy-C11; D, F) were assessed 5 days post infection by flow cytometry. C, D. Representative histograms of three independent experiments (n=3) showing mean fluorescence intensity relative to EV control. E, F Box and whisker plots of pooled MAPKi-R/S data including 3 MAPKi-R and 3 MAPKi-S cell lines transduced with two different shRNAs for S6K1 or S6K2. Median, upper quartile and lower quartile are shown. B, E, F Data are from three independent biological replicates. Kruskal-Wallis ANOVA tests were used to compare groups and corrected for multiple comparisons with Dunn's test.

FIGS. 30A-30H—A. NRAS-mutant melanoma cells (M93-047) were transduced with lentivirus encoding S6K1 shRNA, S6K2 shRNA or non-targeting vector control (Vec) and subjected to LC-MS/MS (A) or reversed phase protein array (RPPA) (B) analysis (4 dpi). A. Schematic depicts the highest ranked proteins differentially regulated by S6K2 vs. S6K1 knockdown, identifying lipid metabolism and redox homeostasis as processes disrupted by S6K2 depletion. Pathways/biological processes for the top proteins were overlaid with IPA canonical pathway analysis, results are color coded. B. Heatmap depicts log 2 mean fold change of 2 shRNA relative to vector for lipid metabolism and oxidative stress response targets in the RPPA dataset. Significant differences between S6K1sh vs. S6K2sh were estimated using paired, two-sided t-tests. C.-G. M93-047 cells were transduced with lentivirus encoding S6K1 or S6K2 shRNA and stained for the lipid peroxidation product 4-hydroxynonenal (4-HNE) (C, E) or the oxidative DNA damage marker 8-OHdG (D, F) 6 dpi. Samples were analyzed by flow cytometry (C-D; mean fluorescence intensity is shown) or immunofluorescence (E-F). G. DNA double strand breaks were assessed by p-gH2AX flow cytometry 6 dpi (mean±SD, n=2). H. Representative Western blots (n=2) showing knockdown of S6K1 or S6K2 (4 dpi) for FIG. 29C-F samples. A, B, G. Statistical significance was assessed by unpaired two-tailed Student's t-tests. A 10% FDR was applied and Benjamini-Hochberg adjusted p values reported (a).

FIGS. 31A-31H—S6K2 depletion activates a terminal unfolded protein response (UPR) and PPARα. MAPKi-R were transduced with S6K1 shRNA, S6K2 shRNA or vector control. A. Cells were analyzed by RNA sequencing (3 dpi). Genes significantly altered by S6K1/2 depletion were analyzed by IPA. Heatmap depicts z-scores for predicted activity of unfolded protein response (UPR) regulators, PPAR genes and PPAR modulators. Positive/red: activated; negative/blue: inhibited by S6K1 or S6K2 shRNA; ns: no significant enrichment. B., C. Endoplasmic reticulum (ER) stress markers were monitored following transduction with shS6K2 or EV control by Western blotting (B; M93-047, 3 dpi) and qRT-PCR (C; M93-047, sh4 3 dpi, sh6 4 dpi; WM1366 4 dpi). D. M93-047 cells transduced with S6K2 shRNA or empty vector (EV) control were treated with the indicated compounds. Cell death was analyzed by Annexin V/PI staining 5 dpi (mean±SD (n=4); unpaired two-sided t-test). E. M93-047 cells transduced with EV/shS6K2 were treated with the IRE1α inhibitor Kira6. Levels of spliced XBP-1 and PPARα were quantified by qRT-PCR (3 dpi, sh4 or 4 dpi, sh6). F, H PPAR gene family (F) and PPAR targets (H) mRNA expression were determined by qRT-PCR 4 dpi with S6K2 shRNA/EV. G. M93-047 cells were transduced with EV, S6K1 or S6K2 shRNA (3 dpi) and analyzed by RNA sequencing. Heatmap depicts mRNA expression levels of selected PPAR targets relative to the mean across samples; p value: Student's t-test. C, E, F, H. Data are shown as mean±SD from three independent experiments. *p≤0.05, **p≤0.01, ***p≤0.001; unpaired two-sided t-tests.

FIGS. 32A-32C—A. M93-047 cells were treated as in FIG. 31D, except media was supplemented with ferrostatin to evaluate ferroptosis dependence. B. S6K2 levels in samples from FIG. 31C were monitored by qRT-PCR. C. qRT-PCR analysis of S6K1 and S6K2 levels from FIG. 31F. A, B, C, Data are mean±SD from three independent experiments. P-values from unpaired two-sided t-tests.

FIGS. 33A-33E—A., B. Interaction of S6K2 with PPARα or NCoR1 was determined by proximity ligation assay (PLA). Representative images. Scale bar=30 μM. C. Bar graphs show quantification of PLA signals for a (top) and b (bottom); mean±SD (n denotes fields quantified). S6K2 Ab, PPARα Ab or NCoR1 Ab indicates single antibody controls. D, E. Transduced cells (4 dpi) were analyzed by PLA. (D) MAPKi-R M93047 cells, (E) MAPKi-S WM3451 cells. Representative images shown, Scale bar=30 μM. F. Bar graphs show quantification of PLA signals for d (top) and e (bottom); mean±SD (n denotes fields quantified).

FIGS. 34A& 34B—A. S6K2 knockdown efficiency was assessed by qRT-PCR (FIG. 33D; 4 dpi); mean±SD (n=2-3) B. S6K2 knockdown efficiency of FIG. 33E (4 dpi) was assessed by western blotting.

FIGS. 35A-35J—A. Relative expression of PPARα in MAPKi-R (red, n=?) and MAPKi-S (blue, n=?) cells determined by immunoblotting (see FIG. 38A), was compared by Student's T-test. Data represent average PPARα levels from two independent experiments. B. AUC for MAPKi-R and MAPKi-S cells (shown in FIG. 38C) were compared by Student's T-test. C. Cells were treated with 50 μM fenofibrate, 7.5 μM DHA, as single agents or in combination for 72 h. Cell death were assessed by Annexin V/PI positivity (mean±SD; n=3; unpaired two-tailed Students t-test). D., E. Cells were treated with 50 μM fenofibrate, 7.5 μM DHA, as single agents or in combination. Lipid peroxidation was assessed by Bodipy-C11. (D) representative histograms and (E) data from three independent experiments showing mean fluorescence intensity relative to vehicle control. F., G. M93-047 cells were pre-treated with the indicated compounds [(F) antioxidant GSH (2 mM), pan-caspase inhibitor (20 μM zVAD) for 24 h or (G) pan-caspase inhibitor (20 μM zVAD), UPR/IRE1 inhibitor 0.2 μM Kira6 for 48 h)] and then treated with 50 μM fenofibrate, and/or 7.5 μM DHA for 24 h. Cell death was analyzed by AnnexinV/PI positivity. Graphs show mean±SD (n=3), individual dots represent independent experiments; unpaired two-sided t-test). H., I. M93-047 cells were treated with 0.2 μM Kira6 for 48 h and then treated with 50 μM fenofibrate, 7.5 μM DHA for 24 h. (H-I) Levels of spliced XBP-1 were quantified by qRT-PCR, mean±SD (n=3); unpaired two-sided t-test or Western blotting (I). J. MAPKi-R M93-047 and WM1366 cells grown as collagen-embedded 3D spheroids were treated with Vehicle (left) 50 μM fenofibrate, 7.5 μM DHA (right) for 5 days. Spheroids were stained with Calcein (AM) (green; live cells) and EtBr (red; dead cells) and imaged with a fluorescence microscope. Representative images of three replicates are shown; the scale bar represents 1000 μM.

FIGS. 36A-36D—A. PPARα levels in MAPKi-R and MAPKi-S cells were determined by western blotting. PPARα levels were normalized to actin; numbers indicate protein levels relative to UACC1273. B. MAPKi-R (red) and MAPKi-S (blue) cells were treated with increasing doses of fenofibrate for 72 h; cell viability was determined by CellTiter-Glo. Data represent average from two independent experiments ±SD. C. Cells were treated with 50 μM fenofibrate, 7.5 μM DHA as single agents or in combination. ROS was assessed by H2DCFDA. Representative histograms (n=3) showing mean fluorescence intensity relative to vehicle control. D. Levels of PPARα were quantified by qRT-PCR mean SD (n=3); unpaired two-sided t test

FIGS. 37A-37D—A. Mice bearing MAPKi-R M93-47-derived subcutaneous tumors were treated with fenofibrate (200 mg/kg), DHA (300 mg/kg) as single agents or in combination. Tumor volume for individual mice (gray lines) or mean (black lines, n=8) is shown. B. C57BI/6j mice bearing MAPKi-R LSL-Nras^{Q61R/Q61R} (TpN^{61R/61R}) syngeneic tumors were treated with vehicle (n=8), fenofibrate (200 mg/kg, n=9), DHA (300 mg/kg, n=8) or fenofibrate+DHA combination (n=8) when tumors reached ~85 mm³ (19 dpi). A-B p-values and FDR adjusted p-values were estimated from a linear mixed-effect model with all follow-up subjects and time points. C., D. Mice bearing PDXs were fed control chow or DHA (0.15% w/w) plus FNB (0.15% w/w) laced chow when tumors reached ~100-150 mm³ (MAPKi-R WM4023, control n=5, DHA+FNB n=4) or (MAPKi-S WM4299-1, control n=5, DHA+FNB n=5). Mice were followed until tumors reached a pre-defined volume (1200 mm³). P values were calculated using log-rank (Mantel-Cox) test.

FIGS. 38A-38C—A. Weight of animals enrolled in the study depicted in FIGS. 37A & 37B at the last follow-up time points (day 24 and 30 respectively). Mice were treated with fenofibrate (200 mg/kg) or DHA (300 mg/kg) as a single agent or in combination. Data are shown as mean±SEM. B., C. Mice bearing WM4023 or WM4229-1 patient derived xenografts (~85 or 70 mm³) were treated with the MEK inhibitor (PD0325901 (1.5 or 5 mg/kg) and classified as MAPKi-resistant PDXs; mean±SEM, n=4 mice/treatment group. FDR adjusted p-values were estimated from a linear mixed-effect model with all follow-up subjects and time points.

FIGS. 39A-39E—Tumors derived from Mice bearing MAPKi-R WM4023 (PDX), or MAPKi-S WM3623 xenografts treated with fenofibrate (200 mg/kg) or DHA (300 mg/kg) as single agents or in combination for 7 days were analyzed by immunohistochemistry for (A) 4-HNE (C) 8-OHdG. A., C. Representative images. Scale bar=25 mm. B., D. Tumors were scored using QuPath software. Bar graphs show H-score from representative fields (Mean±SD; WM4023 n=2, WM3623 n=3) t-test; n.s., not significant. E. Graphical model (created with BioRender.com) depicting MAPKi-R NRAS-mutant melanoma when S6K2 active and present (left panel) and when S6K2 is depleted (right panel).

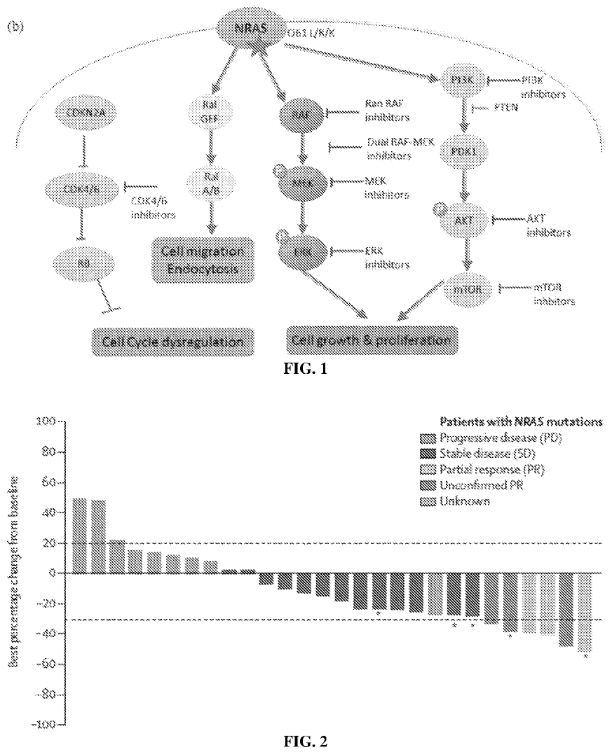

### DESCRIPTION OF ILLUSTRATIVE EMBODIMENTS

Provided herein are compounds and compositions that may be used treat one or more conditions associated with cancers such as melanoma. In particular, described herein are small-molecule inhibitors that could be important leads for the treatment of NRAS^{mut} melanoma. These compounds may target S6K, a Ser/Thr kinase downstream and a node of convergence for both the MAPK and PI3K pathways. S6K consists of two paralogs S6K1 and S6K2, and previous biological studies have established that selective inhibition of S6K2 without suppression of S6K1 leads to melanoma cell death, revealing a therapeutic vulnerability that could be targeted by selective small-molecule inhibitors. An initial screen of 44,000 compounds led to the identification of CD02 as an initial hit. These and more details will be described below.

### I. COMPOUNDS AND FORMULATIONS THEREOF

### A. Compounds of the Present Disclosure

The compounds of the present disclosure are shown, for example, in the summary section above and in Table 1 and the claims below. They may be made using the synthetic methods outlined in the Examples section. These methods can be further modified and optimized using the principles and techniques of organic chemistry as applied by a person skilled in the art. Such principles and techniques are taught, for example, in Smith, March's Advanced Organic Chemistry: Reactions, Mechanisms, and Structure, (2013), which is incorporated by reference herein. In addition, the synthetic methods may be further modified and optimized for preparative, pilot- or large-scale production, either batch or continuous, using the principles and techniques of process chemistry as applied by a person skilled in the art. Such principles and techniques are taught, for example, in Anderson, Practical Process Research & Development—A Guide for Organic Chemists (2012), which is incorporated by reference herein.

TABLE 1
Compounds of the Present Disclosure

| Compound | |
|---|---|
| ID | Structure |
| MM-113 | 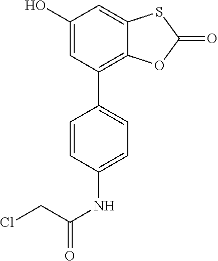 [SMILES: O=C(CCl)Nc1ccc(-c2cc(O)cc3sc(=O)oc23)cc1] |
| RT13 | 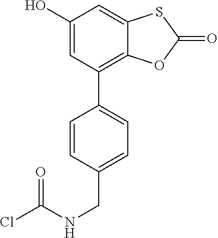 |
| MM-106 | 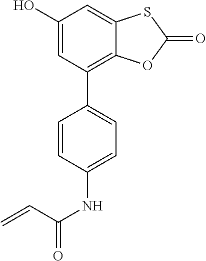 [SMILES: C=CC(=O)Nc1ccc(-c2cc(O)cc3sc(=O)oc23)cc1] |
| RT12 | 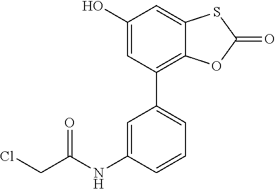 |
| RT14 | 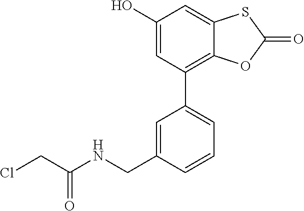 |
| RT10 | 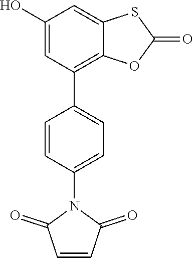 |

All the compounds of the present disclosure may in some embodiments be used for the prevention and treatment of one or more diseases or disorders discussed herein or otherwise. In some embodiments, one or more of the compounds characterized or exemplified herein as an intermediate, a metabolite, and/or prodrug, may nevertheless also be useful for the prevention and treatment of one or more diseases or disorders. As such unless explicitly stated to the contrary, all the compounds of the present disclosure are deemed “active compounds” and “therapeutic compounds” that are contemplated for use as active pharmaceutical ingredients (APIs). Actual suitability for human or veterinary use is typically determined using a combination of clinical trial protocols and regulatory procedures, such as those administered by the Food and Drug Administration (FDA). In the United States, the FDA is responsible for protecting the public health by assuring the safety, effectiveness, quality, and security of human and veterinary drugs, vaccines and other biological products, and medical devices.

In some embodiments, the compounds of the present disclosure have the advantage that they may be more efficacious than, be less toxic than, be longer acting than, be more potent than, produce fewer side effects than, be more easily absorbed than, more metabolically stable than, more lipophilic than, more hydrophilic than, and/or have a better pharmacokinetic profile (e.g., higher oral bioavailability and/or lower clearance) than, and/or have other useful pharmacological, physical, or chemical properties over, compounds known in the prior art, whether for use in the indications stated herein or otherwise.

Compounds of the present disclosure may contain one or more asymmetrically-substituted carbon or nitrogen atom and may be isolated in optically active or racemic form. Thus, all chiral, diastereomeric, racemic form, epimeric form, and all geometric isomeric forms of a chemical formula are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the compounds of the present disclosure can have the S or the R configuration. In some embodiments, the present compounds may contain two or more atoms which have a defined stereochemical orientation.

Chemical formulas used to represent compounds of the present disclosure will typically only show one of possibly several different tautomers. For example, many types of ketone groups are known to exist in equilibrium with corresponding enol groups. Similarly, many types of imine groups exist in equilibrium with enamine groups. Regardless of which tautomer is depicted for a given compound, and regardless of which one is most prevalent, all tautomers of a given chemical formula are intended.

In addition, atoms making up the compounds of the present disclosure are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

In some embodiments, compounds of the present disclosure exist in salt or non-salt form. With regard to the salt form(s), in some embodiments the particular anion or cation forming a part of any salt form of a compound provided herein is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

### B. Pharmaceutical Formulations and Routes of Administration

In another aspect, for administration to a patient in need of such treatment, pharmaceutical formulations (also referred to as a pharmaceutical preparations, pharmaceutical compositions, pharmaceutical products, medicinal products, medicines, medications, or medicaments) comprise a therapeutically effective amount of a compound disclosed herein formulated with one or more excipients and/or drug carriers appropriate to the indicated route of administration. In some embodiments, the compounds disclosed herein are formulated in a manner amenable for the treatment of human and/or veterinary patients. In some embodiments, formulation comprises admixing or combining one or more of the compounds disclosed herein with one or more of the following excipients: lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia, sodium alginate, polyvinylpyrrolidone, and/or polyvinyl alcohol. In some embodiments, e.g., for oral administration, the pharmaceutical formulation may be tableted or encapsulated. In some embodiments, the compounds may be dissolved or slurried in water, polyethylene glycol, propylene glycol, ethanol, corn oil, cottonseed oil, peanut oil, sesame oil, benzyl alcohol, sodium chloride, and/or various buffers. In some embodiments, the pharmaceutical formulations may be subjected to pharmaceutical operations, such as sterilization, and/or may contain drug carriers and/or excipients such as preservatives, stabilizers, wetting agents, emulsifiers, encapsulating agents such as lipids, dendrimers, polymers, proteins such as albumin, nucleic acids, and buffers.

Pharmaceutical formulations may be administered by a variety of methods, e.g., orally or by injection (e.g. subcutaneous, intravenous, and intraperitoneal). Depending on the route of administration, the compounds disclosed herein may be coated in a material to protect the compound from the action of acids and other natural conditions which may inactivate the compound. To administer the active compound by other than parenteral administration, it may be necessary to coat the compound with, or co-administer the compound with, a material to prevent its inactivation. In some embodiments, the active compound may be administered to a patient in an appropriate carrier, for example, liposomes, or a diluent. Pharmaceutically acceptable diluents include saline and aqueous buffer solutions. Liposomes include water-in-oil-in-water CGF emulsions as well as conventional liposomes.

The compounds disclosed herein may also be administered parenterally, intraperitoneally, intraspinally, or intracerebrally. Dispersions can be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. The carrier can be a solvent or dispersion medium containing, for example, water, ethanol, polyol (such as, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

The compounds disclosed herein can be administered orally, for example, with an inert diluent or an assimilable edible carrier. The compounds and other ingredients may also be enclosed in a hard or soft-shell gelatin capsule, compressed into tablets, or incorporated directly into the patient's diet. For oral therapeutic administration, the compounds disclosed herein may be incorporated with excipients and used in the form of ingestible tablets, buccal tablets, troches, capsules, elixirs, suspensions, syrups, wafers, and the like. The percentage of the therapeutic compound in the compositions and preparations may, of course, be varied. The amount of the therapeutic compound in such pharmaceutical formulations is such that a suitable dosage will be obtained.

The therapeutic compound may also be administered topically to the skin, eye, ear, or mucosal membranes. Administration of the therapeutic compound topically may include formulations of the compounds as a topical solution, lotion, cream, ointment, gel, foam, transdermal patch, or tincture. When the therapeutic compound is formulated for topical administration, the compound may be combined with one or more agents that increase the permeability of the compound through the tissue to which it is administered. In other embodiments, it is contemplated that the topical administration is administered to the eye. Such administration may be applied to the surface of the cornea, conjunctiva, or sclera. Without wishing to be bound by any theory, it is believed that administration to the surface of the eye allows the therapeutic compound to reach the posterior portion of the eye. Ophthalmic topical administration can be formulated as a solution, suspension, ointment, gel, or emulsion. Finally, topical administration may also include administration to the mucosa membranes such as the inside of the mouth. Such administration can be directly to a particular location within the mucosal membrane such as a tooth, a sore, or an ulcer. Alternatively, if local delivery to the lungs is desired the therapeutic compound may be administered by inhalation in a dry-powder or aerosol formulation.

In some embodiments, it may be advantageous to formulate parenteral compositions in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical carrier. In some embodiments, the specification for the dosage unit forms of the disclosure are dictated by and directly dependent on (a) the unique characteristics of the therapeutic compound and the particular therapeutic effect to be achieved, and (b) the limitations inherent in the art of compounding such a therapeutic compound for the treatment of a selected condition in a patient. In some embodiments, active compounds are administered at a therapeutically effective dosage sufficient to treat a condition associated with a condition in a patient. For example, the efficacy of a compound can be evaluated in an animal model system that may be predictive of efficacy in treating the disease in a human or another animal.

In some embodiments, the effective dose range for the therapeutic compound can be extrapolated from effective doses determined in animal studies for a variety of different animals. In some embodiments, the human equivalent dose (HED) in mg/kg can be calculated in accordance with the following formula (see, e.g., Reagan-Shaw et al., FASEB J., 22(3):659-661, 2008, which is incorporated herein by reference):

HED (mg/kg)=Animal dosee (mg/kg)×(Animal Kₘ/Human Kₘ)

Use of the Kₘ factors in conversion results in HED values based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are specific to each individual. Nonetheless, a calculated HED dose provides a general guide. Other factors affecting the dose include the physical and clinical state of the patient, the route of administration, the intended goal of treatment and the potency, stability and toxicity of the particular therapeutic formulation.

The actual dosage amount of a compound of the present disclosure or composition comprising a compound of the present disclosure administered to a patient may be determined by physical and physiological factors such as type of animal treated, age, sex, body weight, severity of condition, the type of disease being treated, previous or concurrent therapeutic interventions, idiopathy of the patient and on the route of administration. These factors may be determined by a skilled artisan. The practitioner responsible for administration will typically determine the concentration of active ingredient(s) in a composition and appropriate dose(s) for the individual patient. The dosage may be adjusted by the individual physician in the event of any complication.

In some embodiments, the therapeutically effective amount typically will vary from about 0.001 mg/kg to about 1000 mg/kg, from about 0.01 mg/kg to about 750 mg/kg, from about 100 mg/kg to about 500 mg/kg, from about 1 mg/kg to about 250 mg/kg, from about 10 mg/kg to about 150 mg/kg in one or more dose administrations daily, for one or several days (depending of course of the mode of administration and the factors discussed above). Other suitable dose ranges include 1 mg to 10,000 mg per day, 100 mg to 10,000 mg per day, 500 mg to 10,000 mg per day, and 500 mg to 1,000 mg per day. In some embodiments, the amount is less than 10,000 mg per day with a range of 750 mg to 9,000 mg per day.

In some embodiments, the amount of the active compound in the pharmaceutical formulation is from about 2 to about 75 weight percent. In some of these embodiments, the amount is from about 25 to about 60 weight percent.

Single or multiple doses of the agents are contemplated. Desired time intervals for delivery of multiple doses can be determined by one of ordinary skill in the art employing no more than routine experimentation. As an example, patients may be administered two doses daily at approximately 12-hour intervals. In some embodiments, the agent is administered once a day.

The agent(s) may be administered on a routine schedule. As used herein a routine schedule refers to a predetermined designated period of time. The routine schedule may encompass periods of time which are identical, or which differ in length, as long as the schedule is predetermined. For instance, the routine schedule may involve administration twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc. In other embodiments, the disclosure provides that the agent(s) may be taken orally and that the timing of which is or is not dependent upon food intake. Thus, for example, the agent can be taken every morning and/or every evening, regardless of when the patient has eaten or will eat.

### II. METHODS OF TREATMENT AND COMBINATION THERAPIES

### A. Methods of Treatment

In particular the compositions that may be used in treating a disease or disorder in a subject (e.g., a human subject) are disclosed herein. The compositions described above are preferably administered to a mammal (e.g., rodent, human, non-human primates, canine, bovine, ovine, equine, feline, etc.) in an effective amount, that is, an amount capable of producing a desirable result in a treated subject (e.g., slowing, stopping, reducing or eliminating one or more symptoms or underlying causes of disease). Toxicity and therapeutic efficacy of the compositions utilized in methods of the disclosure can be determined by standard pharmaceutical procedures. As is well known in the medical and veterinary arts, dosage for any one animal depends on many factors, including the subject's size, body surface area, body weight, age, the particular composition to be administered, time and route of administration, general health, the clinical symptoms and other drugs being administered concurrently. In some embodiments, the amount of the compounds used is calculated to be from about 0.01 mg to about 10,000 mg/day. In some embodiments, the amount is from about 1 mg to about 1,000 mg/day. In some embodiments, the compounds may be administered for 1 day to 20 days. In further embodiments, it is contemplated that the compounds may be administered for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, or 20 days, or any range derivable therein. In some embodiments, the compounds may be administered for between 3 and 5 days, inclusive. In some embodiments, the compounds may be administered once. It is also contemplated that in some embodiments, the compounds disclosed herein may be administered two or more times. In some embodiments, these dosings may be reduced or increased based upon the biological factors of a particular patient such as increased or decreased metabolic breakdown of the drug or decreased uptake by the digestive tract if administered orally. Additionally, the compounds may be more efficacious and thus a smaller dose is required to achieve a similar effect. Such a dose is typically administered once a day for a few weeks or until sufficient achieve clinical benefit.

The therapeutic methods of the disclosure (which include prophylactic treatment) in general include administration of a therapeutically effective amount of the compositions described herein to a subject in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, family history, and the like).

### B. Combination Therapies

In addition to being used as a monotherapy, the compounds of the present invention may also find use in combination therapies. Effective combination therapy may be achieved with a single composition or pharmacological formulation that includes both agents, or with two distinct compositions or formulations, administered at the same time, wherein one composition includes a compound of this invention, and the other includes the second agent(s). Alternatively, the therapy may precede or follow the other agent treatment by intervals ranging from minutes to months.

To treat diseases or disorders using the methods and compositions of the present disclosure, one would generally contact a cell or a subject with a compound and at least one other therapy. These therapies would be provided in a combined amount effective to achieve a reduction in one or more disease parameter. This process may involve contacting the cells/subjects with both agents/therapies at the same time, e.g., using a single composition or pharmacological formulation that includes both agents, or by contacting the cell/subject with two distinct compositions or formulations, at the same time, wherein one composition includes the compound and the other includes the other agent. In some embodiments, the compounds of the present disclosure or any therapies used in conjunction with the compounds of the present disclosure may be administered in a less than therapeutically effective dose when used either alone or in combination.

Non-limiting examples of such combination therapy include combination of one or more compounds of the invention with another anti-inflammatory agent, an immunosuppressant agent, a chemotherapeutic agent, radiation therapy, an antidepressant, an antipsychotic agent, an anticonvulsant, a mood stabilizer, an anti-infective agent, an antihypertensive agent, a cholesterol-lowering agent or other modulator of blood lipids, an agent for promoting weight loss, an antithrombotic agent, an agent for treating or preventing cardiovascular events such as myocardial infarction or stroke, an antidiabetic agent, an agent for reducing transplant rejection or graft-versus-host disease, an anti-arthritic agent, an analgesic agent, an anti-asthmatic agent or other treatment for respiratory diseases, or an agent for treatment or prevention of skin disorders. Compounds of the invention may be combined with agents designed to improve a patient's immune response to cancer, including (but not limited to) cancer vaccines. See Lu et al. (2011), which is incorporated herein by reference.

Cancer therapies also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein transferase inhibitors, transplatinum, 5-fluorouracil, vincristine, vinblastine and methotrexate, Temazolomide (an aqueous form of DTIC), or any analog or derivative variant of the foregoing. The combination of chemotherapy with biological therapy is known as biochemotherapy. The present invention contemplates any chemotherapeutic agent that may be employed or known in the art for treating or preventing cancers.

In particular, the present disclosure relates to the use of compounds of the present disclosure with one or more inhibitors of histone demethylase. In particular, the present disclosure contemplates that the compounds may be used in combination with either lysine-specific histone demethylase 1A (LSD1), lysine (K)-specific demethylase 1A (KDM1A), or lysine-specific demethylase 4A (KDM4A). In particular, the compounds of the present disclosure may be used with an inhibitor such as a small molecule inhibitor, an antibody, or a nucleic acid. Some examples of histone demethylase inhibitors can be found such as in Kaniskan et al., 2018, which is incorporated by reference.

Other factors that cause DNA damage and have been used extensively include what are commonly known as 7-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic agent and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

It is also conceivable that more than one administration of either the compound or the other therapy will be desired. Various combinations may be employed, where a compound of the present disclosure is “A,” and the other therapy is “B,” as exemplified below:
A/B/A B/A/B B/B/A A/A/B B/A/A A/B/B B/B/B/A B/B/A/B A/A/B/B A/B/A/B A/B/B/A B/B/A/A B/A/B/A B/A/A/B B/B/B/A A/A/A/B B/A/A/A A/B/A/A A/A/B/A A/B/B/B B/A/B/B B/B/A/B
   Other combinations are also contemplated.

### III. Melanoma

In some embodiments, the compounds and compositions of the present disclosure may be used to treat melanoma. Melanoma, also known as malignant melanoma, is a type of skin cancer that develops from the pigment-producing cells known as melanocytes. Melanomas typically occur in the skin, but may rarely occur in the mouth, intestines, or eye (uveal melanoma). In women, they most commonly occur on the legs, while in men, they most commonly occur on the back. About 25% of melanomas develop from moles. Changes in a mole that can indicate melanoma include an increase in size, irregular edges, change in color, itchiness, or skin breakdown.

The primary cause of melanoma is ultraviolet light (UV) exposure in those with low levels of the skin pigment melanin. The UV light may be from the sun or other sources, such as tanning devices. Those with many moles, a history of affected family members, and poor immune function are at greater risk. A number of rare genetic conditions, such as xeroderma pigmentosum, also increase the risk. Diagnosis is by biopsy and analysis of any skin lesion that has signs of being potentially cancerous.

Melanomas are graded according to the deepest depth to which they penetrate the skin tissue. The depth may be described in terms of millimeters (Breslow) or by the depth level of various structures in the skin tissue to which the melanoma has penetrated. Melanoma is curable in its earliest phases and can arise on its own or from an atypical or unusual mole. More than 90% of melanomas occur on the visible portions of the body, that is on the skin.

The incidence and mortality rates of malignant melanoma continues to rise dramatically throughout the World. In the United States, it is estimated that nearly one in 90 Americans will develop melanoma. Melanoma is one of the most feared neoplasms because of the high mortality associated with metastasis. Melanomas usually metastasize first via the lymphatic system, with involvement of regional nodes, and then via blood vessels, with dissemination to subcutaneous tissue and to the liver, lungs, and brain. The presence of regional lymph node metastasis is predictive of a poor prognosis.

As set forth, melanomas arise from melanocytes and are typically pigmented (melanotic) due to accumulation of melanin, which imparts a dark color to melanomas. Some melanocytes may be less well differentiated and therefore produce little or no melanin. Melanomas arising from melanocytes can be nonpigmented or amelanotic. Approximately 70 percent of melanomas are of the superficial spreading type. Generally, this type of melanoma is characterized by a pre-existing mole that slowly changes over a period of one to five years which is then followed by a period of rapid changes close to the time that the melanoma is diagnosed. Typically, this type of melanoma first appears as a very dark area in an existing mole. As the pre-melanoma enlarges, the edges usually appear notched or indented. Superficial spreading melanoma may appear any-time after puberty and is seen more often in women than in men.

Nodular melanoma is the second most common type of melanoma, accounting for approximately 15 to 30 percent of cases. These melanomas usually do not appear in an existing nevi. Instead, they start as melanomas and are often darker in color than the superficial spreading type. Nodular melanomas typically take on a blue-black appearance and look like blood blisters. Some may have shades of purple, gray or red in them, or lack color and have a fleshy appearance. They are usually 1 cm to 2 cm or larger in diameter. Nodular melanomas appear more often in men and are usually seen in middle age, although they may appear at any age.

A small percentage of melanomas, 4 to 10 percent, are lentigo maligna melanomas. They differ greatly from other types in that they usually do not metastasize. They are seen most often on the face and neck, are typically large (greater than 3 cm) and are tan-colored with shades of brown. They appear most often in Caucasian women who are over the age of 50. Acral lentiginous melanomas generally occur on the palms of the hand or the soles of the feet. They may also be seen under a fingernail or toenail. Their incidence is low in Caucasians (only 2 to 8 percent of those having melanoma), but substantially higher among darker-skinned individuals with melanoma (approximately 35 to 60 percent). They appear on average over a two- and one-half-year period and resemble a flat tan or brown stain. It is common for this type of melanoma to break into a sore. Acral lentiginous melanoma is usually seen in persons who are over the age of 60. Physicians currently use CT scans, radionuclide bone scans, liver and spleen scintigraphy, and magnetic resonance imaging to evaluate the extent of melanoma. Usually these tests are conducted when specific symptoms require investigation. However, a biopsy is currently the only recognized definitive means of identifying the melanoma.

Melanoma usually occurs in adults, but it may occasionally be found in children and adolescents. Men most often get melanoma on the trunk (the area of the body between the shoulders and hips) shoulders and hips) or on the head or neck, and women most often get melanoma on the arms and legs. In rare cases, it can develop in parts of the body not covered by the skin, such as the eyes, mouth, large intestine, or vagina.

Melanoma can be difficult to cure, and is often lethal. In the U.S. there are about 38,000 new cases of melanoma a year and approximately 7,000 melanoma deaths occur every year in the U.S. Current therapy for melanoma has many deficiencies and drawbacks. The first line therapy of choice is to remove the melanoma tissue as well as a substantial area of tissue surrounding the melanoma so as to also remove precancerous, premelanoma cells which typically exist in proximity to the melanoma. Pre-melanoma cells are those melanocytes in proximity to a melanoma, such as, for example, a lentigo maligna melanoma, which while not malignant, are irregular and/or atypical melanocytes which if left untreated often become malignant over time. Surgical removal of the melanoma and surrounding tissue results in scaring and the removal of a large areas of tissue can necessitate use of skin grafts. Additionally, where a facial melanoma is removed a highly undesirable cosmetic out-come can result due to the scaring and discoloration of the new skin graft.

Early treatment of melanoma is highly desirable for patient prognosis and there is a need for an effective treatment of melanoma while it is still a pre-melanoma condition. Additionally, there is a need for an effective treatment of pre-melanoma cells in the vicinity of excised melanoma tissue. Thus it is very preferable to treat melanoma in the pre-melanoma stage. Treatment of melanoma at the pre-melanoma stage may greatly increase the prognosis for a patient. Therefore, what is needed are effective methods to treat pre-melanoma. While it is known to treat certain cancerous tissues with PDT and to treat discernable and superficial precancerous tissues (such as AKs) by PDT, there exists no PDT therapy for precancerous pre-melanoma tissues. Note that pre-melanoma cannot be discerned visual or by a tactile examination.

### A. Acral Melanoma

Acral lentiginous melanoma (ALM) is a particular type of malignant melanoma, which generally occurs on the palms or soles of the hands and feet. The word “lentiginous” in the melanoma type means that the spot of melanoma is much darker than the surrounding skin. Generally, ALM or simply AM also has a sharp border between the dark skin and the lighter skin around it. This contrast in color is one of the most noticeable symptoms of this type of melanoma.

While ALM can occur in a person of any ALM is the most common type of melanoma in people with darker skin and those of Asian descent. ALM may be hard to recognize at first, when the patch of darkened skin is small and looks like little more than a stain or bruise.

Generally, the most common symptom of AML is a dark spot of skin that remains the normal skin color with a clear border between the dark skin and the lighter skin around it. These spots are generally found on the hands and feet or the spots are found in the nail beds. While the spots are generally dark, the spots can also be red or orange in color, which are referred to as amelanotic. Common melanoma signs can be used to determine the presence of melanoma: ABCDE.
Asymmetry: The two halves of the spot are not the same as each other, meaning that they may vary in size or shape. Non-cancerous moles are usually round in shape or are the same size and shape on both sides.
Border irregularity: The border around the spot is uneven or jagged. Non-cancerous moles usually have borders that are straight, clearly defined, and solid.
Color variation: The spot is made up of areas of multiple colors of brown, blue, black, or other similar colors. Non-cancerous moles are normally just one color (usually brown).
Large diameter: The spot is larger than a quarter of an inch (0.25 inch, or 6 millimeters) around. Non-cancerous moles are typically much smaller.
Evolving: The spot has gotten bigger or has more colors than when it originally appeared on your skin. Non-cancerous moles usually don't grow or change color as drastically as a spot of melanoma.

Generally, AML starts out smooth and often become bumpier or rougher as it evolves and grows. It may also grow under a fingernail or toenail, which is known as subungual melanoma. In subungual acral melanomas, the nail itself may also be discolored or have lines of discolored, which is known as Hutchinson's sign.

Acral lentiginous melanoma (ALM) or simply acral melanoma (AM) is typically not associated with UV exposure. This type of melanoma generally is associated with genetic mutations or with traumatic injuries.

i. Biological Understanding of ALM/AM

Skin from acral sites is unique and shows striking differences from skin from other anatomical sites, including the absence of hair follicles and sebaceous glands, a compact, thick stratum corneum, hypopigmentation and the presence of encapsulated sense organs. Another peculiarity of acral skin is the presence of a grooved surface continuously alternating ridges and sulci, with eccrine ducts opening at the center of the ridges (Calonje, Brenn, Lazar, & Mckee, 2011). The number of melanocytes in different areas of the body varies substantially (Szabo, 1954). In acral skin of palms and soles, between 40 and 270 melanocytes per 1 mm length of the epidermal/dermal junction are observed. In the nail bed, this number is reduced to less than 20 melanocytes per 1 mm length of the epidermal/dermal junction (Fernandez-Flores & Cassarino, 2017). Melanocytes in the acral skin are normally located in the two lowest layers of the epidermis, in single units, equidistant from each other. The origin of adult melanocytes on acral skin appears to be different from that of melanocytes on non-glabrous sites. All melanocyte precursors are derived from the neural crest, where melanoblasts originate and migrate to colonize the developing epidermis. In non-glabrous skin, they are mainly detected in hair follicles, where they can differentiate into melanocytes or remain as melanocyte stem cells (Gola, Czajkowski, Bajek, Dura, & Drewa, 2012; Lang, Mascarenhas, & Shea, 2013). Differentiated melanocytes have also been identified in sebaceous glands, but whether these differentiate from resident melanoblasts or from other sources such as hair follicle precursors remains to be demonstrated (Jang, Kim, Lee, et al., 2014). In glabrous skin, melanocyte precursors have been detected in the secretory portion of eccrine glands, providing a source of differentiated melanocytes to the epidermis (Okamoto et al., 2014). Interestingly, melanoblasts harboring CCND1 amplification have been detected in the secretory portion of eccrine glands among human melanoma cells, suggesting that these cells could originate acral melanomas. CCND1 encodes Cyclin D1, a protein that forms a complex with cyclin-dependent kinase 4 (CDK4) or 6 (CDK6), and whose activity is required for G1/S transition. This cell distribution may explain the characteristic parallel ridge pattern of acral melanoma (Okamoto et al., 2014). Epidermal appendages are not the only source of melanocytes. Glover and colleagues revealed the presence of distinct populations of melanocytes in the epidermis of mice lacking hair follicles and eccrine glands. These were characterized as pigment-producing melanocytes with proliferative potential interspersed with a small percentage of amelanotic melanocytes, suggesting that these populations could serve as melanocyte reserves (Glover et al., 2015). Another alternative source of melanocytes already described in glabrous skin is the dermis. When embedded in the dermal compartment of skin reconstructs, low-affinity nerve growth factor receptor (NGFR)-positive dermal stem cells isolated from glabrous human foreskin differentiate into melanocytes and migrate from the dermis to the epidermis, aligning singly along the basal layer similar to what would be expected for melanocytes of non-glabrous skin (Li et al., 2010). These results were confirmed by Kumar, Parsad, Rani, Bhardwaj, and Srivastav (2016), who isolated NGFR-positive dermal stem cells from glabrous skin of vitiligo patients and demonstrated that these cells can be differentiated into melanocytes in vitro (Kumar et al., 2016). NGFR has been used as a marker for the isolation of neural crest stem cells, given that NGFR-positive cells have self-renewal capacity and display multipotent differentiation properties (Li et al., 2010). Whether this could also be a source of melanocytes of other anatomical locations remains to be addressed. Apart from melanocyte differentiation, other major differences between acral and non-acral skin relate to the cells and factors that constitute the tissue microenvironment. Skin from palms and soles, for instance, shows distinct gene expression profiles compared with non-acral skin including enhanced expression of the genes encoding keratin 9, 6 and 16 (Bissonnette et al., 2016). Interestingly, keratin 9 is exclusively expressed in acral skin, and its downregulation leads to hyperpigmentation of plantar acral skin in mice, suggesting a role of the microenvironment in regulating melanogenesis and, perhaps, impacting melanocyte function and/or proliferation (Fu et al., 2014). A decrease in CCL27 gene expression was also observed in acral skin (Bissonnette et al., 2016). CCL27 is a chemokine produced by keratinocytes that attracts T cells to the skin, where they play a role in inflammation (Homey et al., 2002). Given the well-studied role of the microenvironment in melanoma development, progression and therapy response, it is conceivable to speculate that melanomas arising in acral locations may be influenced by these microenvironment differences. But cancer development is also influenced by external environmental factors, which act differently according to the anatomical region. For instance, both trauma and UV can influence the structure and composition of the skin, including by generating an inflammatory environment (Liu, Zhang, Gao, & Li, 2016). Interestingly, cutaneous inflammation mediated by mast cells is sustained for longer in hairy skin compared with glabrous skin after chronic exposure to stress, illustrating how external factors may have different impacts depending on the type of skin (reviewed in Căruntu, Boda, Musat, Căruntu, & Mandache, 2014). When looking at the characteristics of the immune infiltrate, the microenvironment can exhibit an immune desert (immunological ignorance), immune excluded or inflamed phenotype. Their cellular and molecular properties are determinants of melanoma progression and therapeutic response (Chen & Mellman, 2017). Differences in the presence of immune cells in ALM compared with other cutaneous melanoma subtypes have also been reported. It has been recently demonstrated that ALM presents increased numbers of M2 macrophages in both peritumoral and intratumoral areas compared with SSM and this was significantly associated with several histopathological characteristics predictive of adverse prognosis including tumor thickness, ulceration, mitotic rate and metastasis (Zúñiga-Castillo, Pereira, & Sotto, 2018). Regarding tumor-infiltrating lymphocytes (TILs), lower levels were associated with ALM compared with other cutaneous melanomas, as well as with higher Breslow, Clark levels and longer overall survival (Castaneda et al., 2017). In addition, an association between lower levels of the tumor-suppressor protein, p16(INK4A) in tumor cells, and lower density of CD3+ and CD8+ TILs was found, suggesting a probable relationship between p16(INK4A) and TIL immune response in ALM (Castaneda et al., 2019). p16(INK4A) is involved in the tumor cell intrinsic regulation of cell cycle progression by blocking the activity of CDK4/6. Park and Kim (2017), however, found no association between the different cutaneous melanoma subtypes and the levels of TILs, but observed that high levels of peritumoral and intratumoral lymphocytes were associated with less recurrence and metastasis and with longer disease-free survival only in ALM.

ii. Genetic Markers of ALM/AM

Elucidating the biological pathways involved in ALM pathogenesis is key to the development of new molecular-based targeted therapies (Shim et al., 2017). Unfortunately, compared with CM, very few molecular studies have been performed on ALM tumors. Although limited, this body of literature already suggests compelling molecular differences between ALM and other melanoma subtypes.

### A. Genetic Drivers

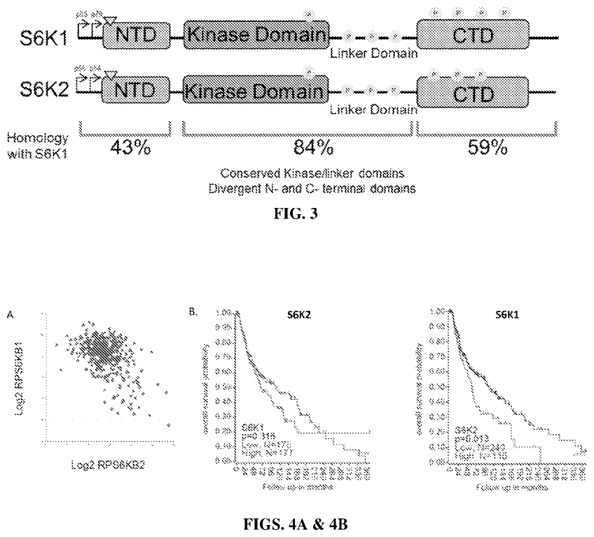

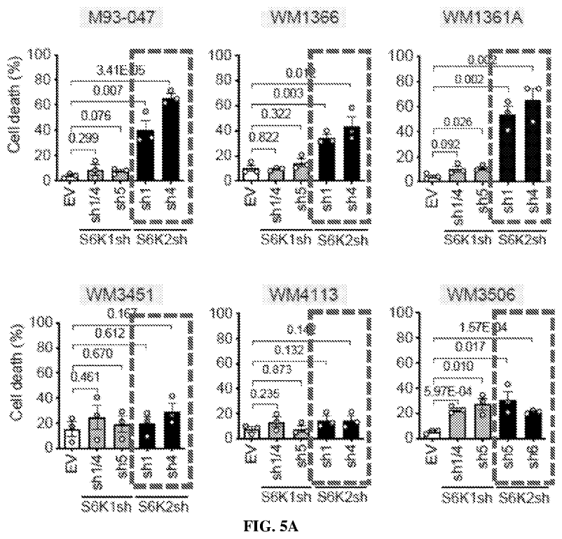

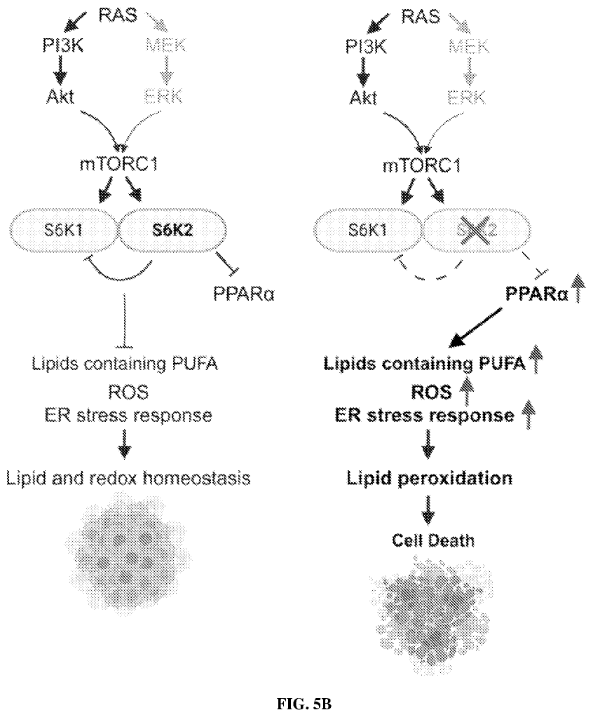

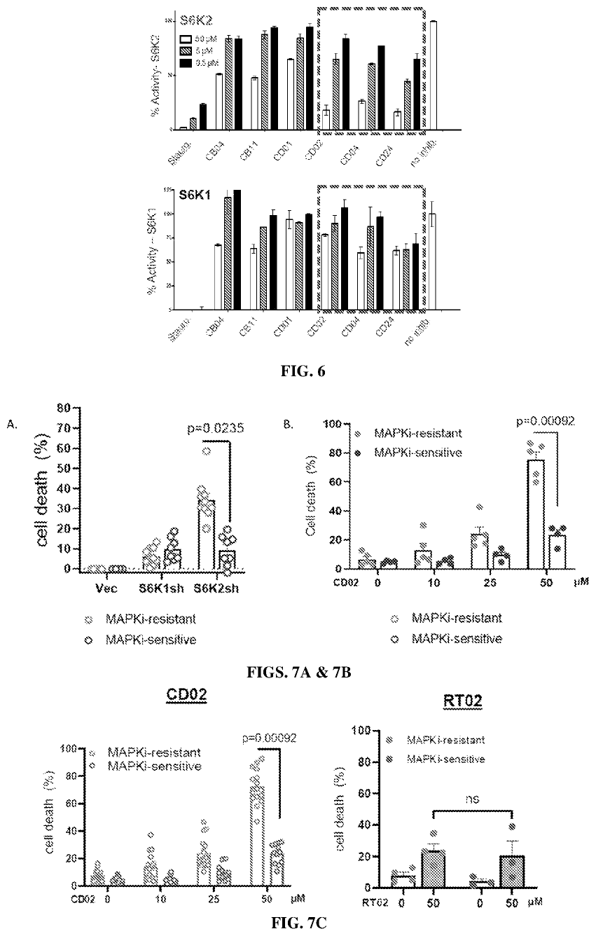

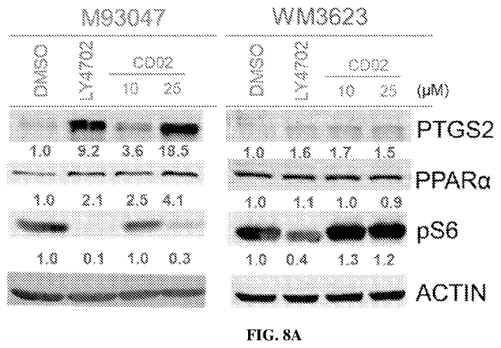

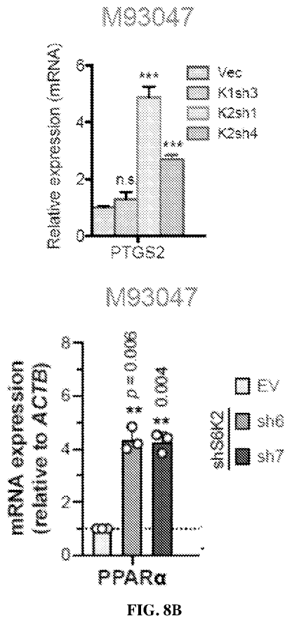

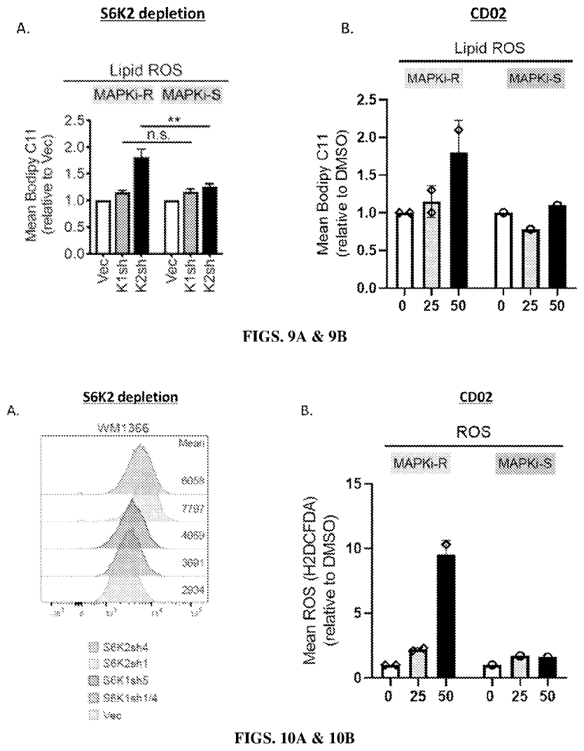

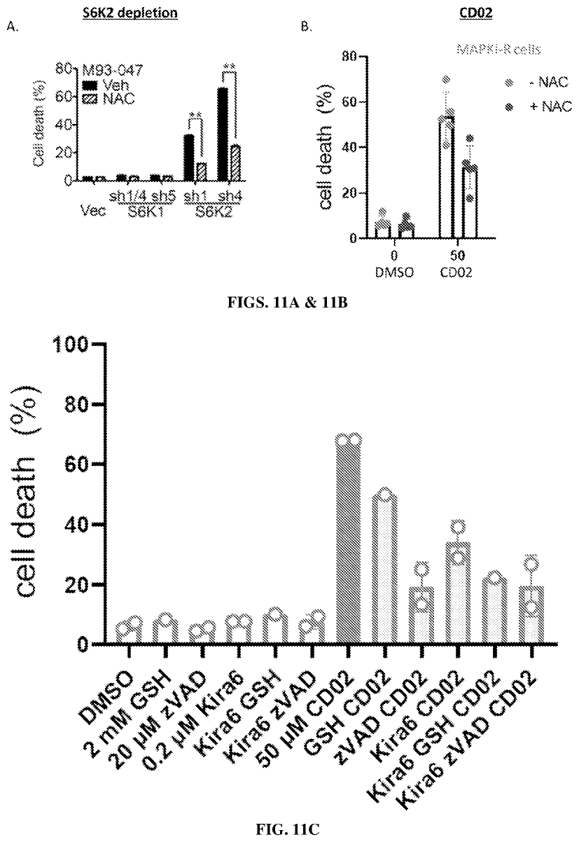

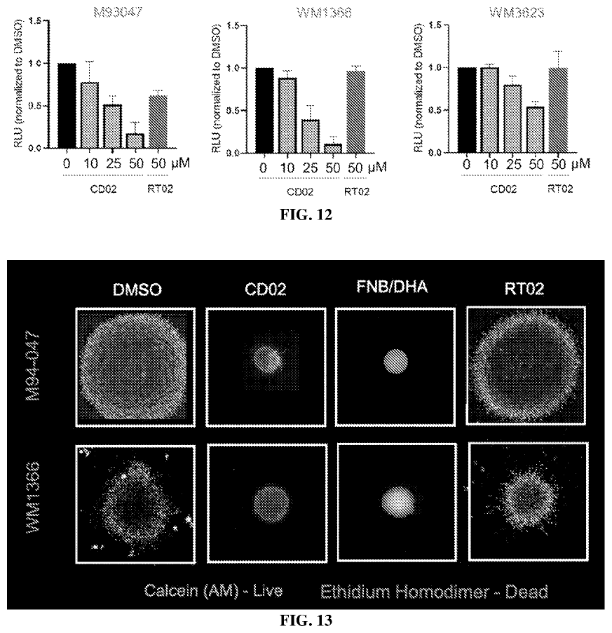

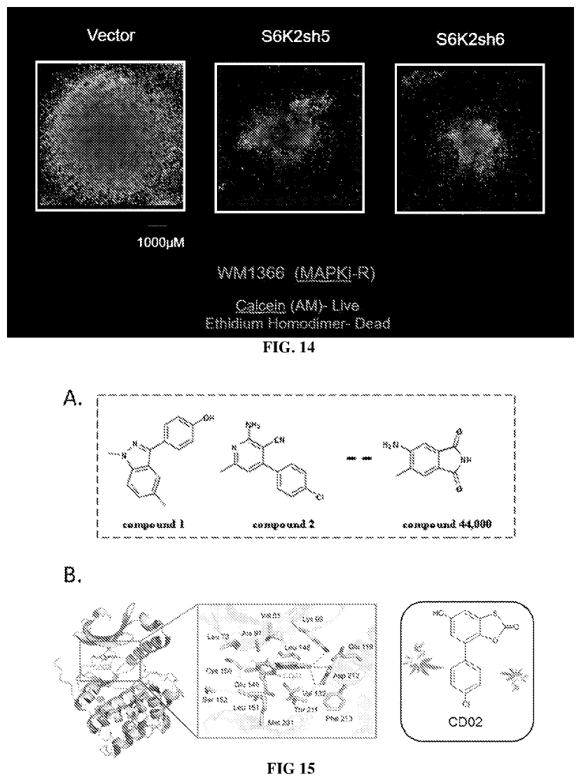

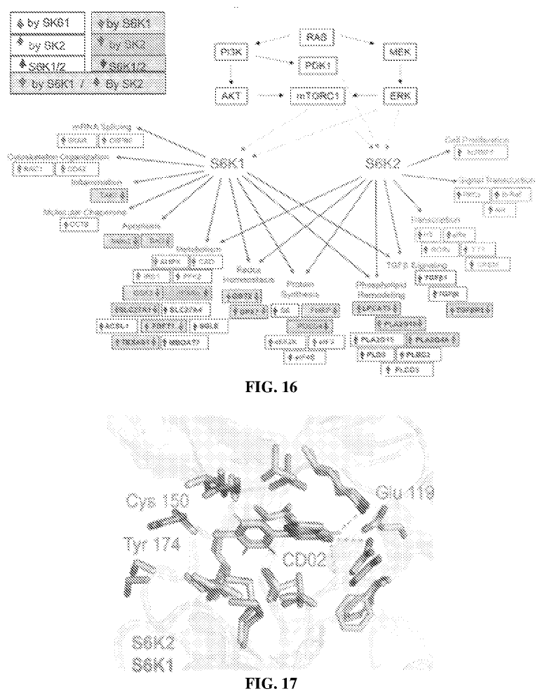

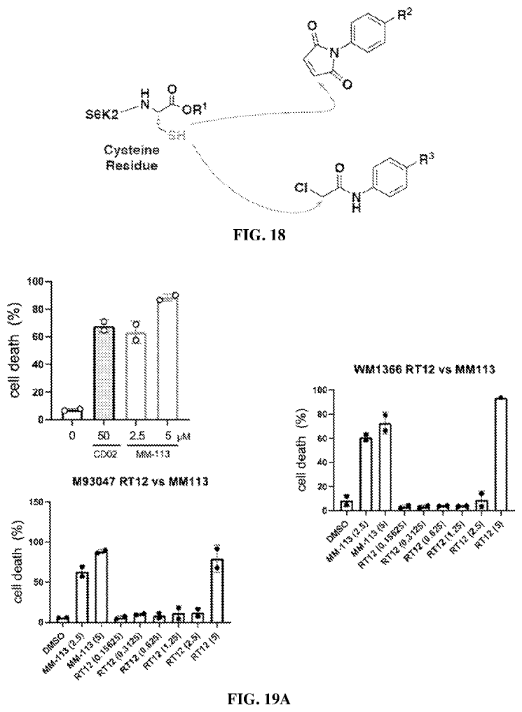

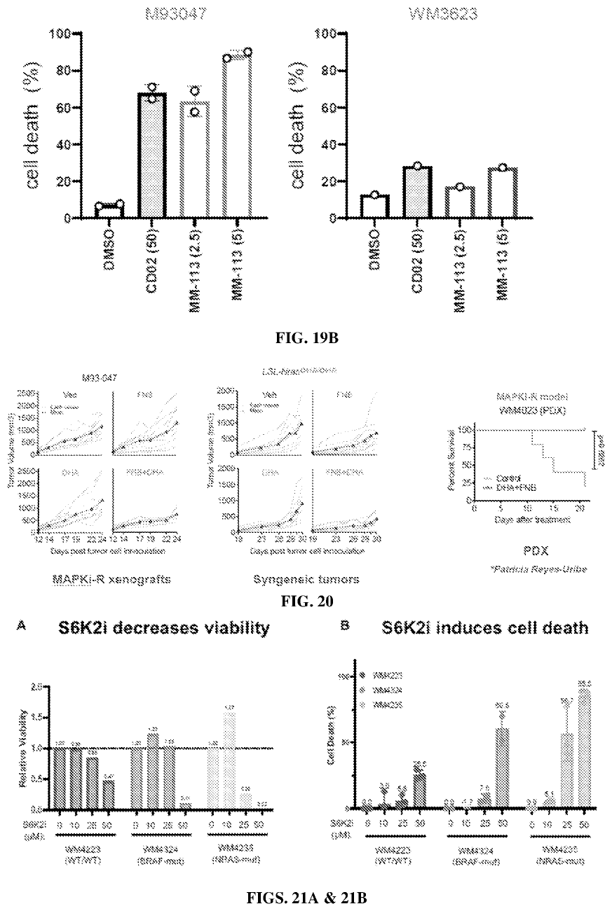

Sequencing and copy-number profiling studies of ALM tumors have identified characteristic tumor-promoting mutations implicating several genes. Examples of these include the KIT proto-oncogene (KIT), which encodes a type III receptor tyrosine kinase that can stimulate cell growth, division, survival and migration; Cyclin D1 (CCND1) and cyclin-dependent kinase 4 (CDK4), which encode a cyclin and a kinase that when bound to each other promote the transition from G1 to S phase; cyclin-dependent kinase inhibitor 2A (CDKN2A), which encodes for two proteins: p16(INK4A) and p14(ARF), the latter of which helps stabilize TP53; telomerase (TERT), encoding an enzyme crucial for telomere maintenance; and aurora kinase A (AURKA), which is involved in regulation of cell cycle progression by playing critical roles in mitosis (Curtin, Busam, Pinkel, & Bastian, 2006; Hayward et al., 2017; Puig-Butillé) In this regard, a study over 514 ALM samples identified that these tumors frequently display a dysregulated CDK4 pathway, a product of either gains of CDK4/CCND1 or loss of CDKN2A that, in turn, promote G1 to S cell cycle transition and thus contribute to tumor proliferation (Kong et al., 2017). Further, as in other melanoma subtypes, TERT or its promoter are frequently mutated; for example, Liang et al. (2017) observed TERT aberrations in 41% of their patients with acral melanoma. In their study, Yeh et al. (2019) observed a low frequency of TERT promoter mutations but amplification of the TERT locus in 10.3% of the cases, and 3.3% cases with copy-number transitions with relative gain of TERT, although the comprehensive role of telomere regulation specific to ALM has not been clarified (Hayward et al., 2017; Liang et al., 2017). ALM stands out from other melanoma subtypes as it carries a large number of structural variants including duplications, deletions, translocations and inversions. Through whole-genome sequencing of 35 ALM and 8 mucosal melanoma (MM) samples, Hayward et al. (2017) identified that, compared with any other melanoma subtype, both ALM and MMs carry significantly higher numbers of genomic rearrangements, in accordance with previous studies (Curtin et al., 2005). Furthermore, these alterations show evidence of being caused by repeated events of breakage-fusion-bridge (BFB) and chromothripsis, and were found in ~68% of the acral samples studied. Such catastrophic events, although found in other cancer types, rarely reach the prevalence found in ALM, which highlights the uniqueness of the molecular etiology of this disease (Rode, Maass, Willmund, Lichter, & Ernst, 2016). Most studies of the molecular profile of ALM have been done in European-descent populations, but there are a few from other populations available. A study from Shim et al. (2017) analyzed the mutational profile of acral melanomas in a Korean population and reported mutational frequencies of 8.5%, 4.3% and 6.4% of KIT, NRAS proto-oncogene (NRAS) and the BRAF proto-oncogene (BRAF), respectively, and noted that their results were similar to those described in other Korean and Asian populations, but that mutation rates were lower than those observed in other studies. As NRAS and BRAF are among the most commonly mutated genes in other melanoma subtypes due to their crucial roles in triggering the mitogen-activated protein kinase (MAPK) pathway, Shim et al. (2017) speculates that the differences between the mutation rates could reflect a different genetic and/or environmental cause between Asian and European populations. Overall, the frequency of RAS, BRAF or neurofibromin 1 (NF1) mutations resulting in the activation of the MAPK pathway seems to be lower in ALM than in other subtypes of melanoma (Hayward et al., 2017; Vazquez et al., 2015; Yun et al., 2011), and other drivers such as KIT, PAK1 and SPRED1 may be more important (Curtin et al., 2006; Liang et al., 2017; Yeh et al., 2019) (FIG. 3). NF1 and SPRED1 encode proteins involved in the regulation of the MAPK signaling pathway, while PAK1 codes for a protein kinase involved in signaling pathways important for cell migration, proliferation, apoptosis, and mitosis. However, a significant proportion of tumors still remain with no known drivers. These studies also suggest that chromosomal instability is a contributing factor to the etiology of ALM and that it is different from that of melanomas in sun-exposed skin.

### B. Mutational Signatures

The identification of mutational signatures from a collection of cancer genomes can pinpoint the processes, both environmental and endogenous, that were operative during tumor development and consequently that caused mutation accumulation. As expected, several mutational signature analyses on CM have identified a high prevalence of the UV-induced signature dominated by C>T transitions (termed Signature 7) (Alexandrov et al., 2013). Unfortunately, few whole ALM genomes and exomes have been analyzed in this way, as this subtype of melanoma is not well represented in some of the largest tumor collections such as The Cancer Genome Atlas (TCGA) (Cancer Genoma Atlas Network, 2015; Hayward et al., 2017), which only has 2 out of 470 samples classified as ALM. From the available analysis, however, we can gather that the dominant signature is not UV-induced, as expected, but rather that two other signatures dominate the ALM mutational landscape, both of which are age-related (Signatures 1 and 5) (Hayward et al., 2017). Regarding structural rearrangements, a recent study that analyzed 256 cutaneous melanomas observed that acral melanomas had an enrichment of a type of genomic rearrangement that the authors named “tyfonas” (Greek word meaning typhoon), which are extremely large amplicons comprising >100 Mb of genomic material and present in high copy number (often in more than 50 copies) (Hadi et al., 2019). It is speculated that these rearrangements may be able to generate neoantigens, explaining the similar response rate to immune checkpoint inhibition therapy between acral and cutaneous melanoma (Shoushtari et al., 2016). However, these conclusions were derived from a small number of tumors; therefore, the knowledge of the molecular events leading to ALM remains limited.

### IV. CHEMICAL DEFINITIONS

When used in the context of a chemical group: “hydrogen” means —H; “hydroxy” means —OH; “oxo” me [symbol] s=O; “c [symbol] bonyl” me [symbol] s —C(═O)—; “c [symbol] boxy” me [symbol] s —C(═O)OH ( [symbol] so written as —COOH or —CO₂H); “halo” means independently —F, —Cl, —Br or —I; “ [symbol] ino” me [symbol] s —NH₂; “hydroxyamino” means —NHOH; “nitro” me [symbol] s —NO₂; imino means ═NH; “cyano” means —CN; “isocy [symbol] yl” me [symbol] s —N═C═O; “ [symbol] ido” me [symbol] s —N₃; in a monovalent context “phosphate” means —OP(O)(OH)₂ or a deprotonated form thereof; in a divalent context “phosphate” means —OP(O)(OH)O— or □deproton [symbol] ed form thereof, “merc [symbol] to” me [symbol] s —SH; and “thio” means ═S; “thiocarbonyl” means —C(═S)—; “sulfonyl” me [symbol] s —S(O)₂—; [symbol] d “sulfinyl” means —S(O)—.

In the context of chemical formulas, the symbol “—” means a single bond, “═” means a double bond, and “≡” means triple bond. The symbol “----” represents an optional bond, which if present is either single or double. The symbol “ [symbol] ” represents a single bond or a double bond. Thus, the formula

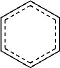

covers, for example

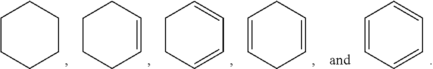

And it is understood that no one such ring atom forms part of more than one double bond. Furthermore, it is noted that the covalent bond symbol “—”, when connecting one or two stereogenic atoms, does not indicate any preferred stereochemistry. Instead, it covers all stereoisomers as well as mixtures thereof. The symbol “ [symbol] ”, when drawn perpendicularly across a bond

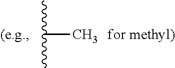

indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a singe bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a variable is depicted as a “floating group” on a ring system, for example, the group “R” in the formula:

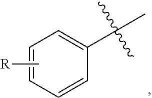

then the variable may replace any hydrogen atom attached to any of the ring atoms, including a depicted, implied, or expressly defined hydrogen, so long as a stable structure is formed. When a variable is depicted as a “floating group” on a fused ring system, as for example the group “R” in the formula:

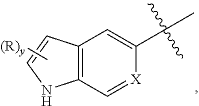

then the variable may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the R enclosed in parentheses, represents a numeric variable. Unless specified otherwise, this variable can be 0, 1, 2, or any integer greater than 2, only limited by the maximum number of replaceable hydrogen atoms of the ring or ring system.

For the chemical groups and compound classes, the number of carbon atoms in the group or class is as indicated as follows: “Cn” or “C=n” defines the exact number (n) of carbon atoms in the group/class. “C≤n” defines the maximum number (n) of carbon atoms that can be in the group/class, with the minimum number as small as possible for the group/class in question. For example, it is understood that the minimum number of carbon atoms in the groups “alkyl_{(C≤8)}”, “cycloalkanediyl_{(C≤8)}”, “heteroaryl_{(C≤8)}”, and “acyl_{(C≤8)}” is one, the minimum number of carbon atoms in the groups “alkenyl_{(C≤8)}”, “alkynyl_{(C≤8)}”, and “heterocycloalkyl_{(C≤8)}” is two, the minimum number of carbon atoms in the group “cycloalkyl_{(C≤8)}” is three, and the minimum number of carbon atoms in the groups “aryl_{(C≤8)}” and “arenediyl_{(C≤8)}” is six. “Cn-n′” defines both the minimum (n) and maximum number (n′) of carbon atoms in the group. Thus, “alkyl_{(C2-10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C5 olefin”, “C5-olefin”, “olefin_{(C5)}”, and “olefin_{C5}” are all synonymous. Except as noted below, every carbon atom is counted to determine whether the group or compound falls with the specified number of carbon atoms. For example, the group dihexylamino is an example of a dialkylamino_{(C=12)} group; however, it is not an example of a dialkylamino_{(C=6)} group. Likewise, phenylethyl is an example of an aralkyl_{(C=8)} group. When any of the chemical groups or compound classes defined herein is modified by the term “substituted”, any carbon atom in the moiety replacing the hydrogen atom is not counted. Thus methoxyhexyl, which has a total of seven carbon atoms, is an example of a substituted alkyl_{(C=6)}. Unless specified otherwise, any chemical group or compound class listed in a claim set without a carbon atom limit has a carbon atom limit of less than or equal to twelve.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” signifies that the compound or chemical group so modified is an acyclic or cyclic, but non-aromatic compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” signifies that the compound or chemical group so modified has a planar unsaturated ring of atoms with 4n+2 electrons in a fully conjugated cyclic π system. An aromatic compound or chemical group may be depicted as a single resonance structure; however, depiction of one resonance structure is taken to also refer to any other resonance structure. For example:

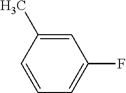

is also taken to refer to

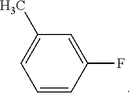

Aromatic compounds may also be depicted using a circle to represent the delocalized nature of the electrons in the fully conjugated cyclic 71 system, two non-limiting examples of which are shown below:

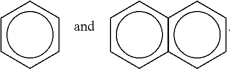

The term “alkyl” refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂ (i-Pr, ⁱPr or isopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tert-butyl, t-butyl, t-Bu or ^{t}Bu), and —CH₂C(CH₃)₃ (neo-pentyl) are non-limiting examples of alkyl groups. The term “alkanediyl” refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂— (methylene), —CH₂CH₂—, —CH₂C(CH₃)₂CH₂—, and —CH₂CH₂CH₂— are non-limiting examples of alkanediyl groups. The term “alkylidene” refers to the divalent group ═CRR′ in which R and R′ are independently hydrogen or alkyl. Non-limiting examples of alkylidene groups include: ═CH₂, ═CH(CH₂CH₃), and ═C(CH₃)₂. An “alkane” refers to the class of compounds having the formula H-R, wherein R is alkyl as this term is defined above.

The term “cycloalkyl” refers to a monovalent saturated aliphatic group with a carbon atom as the point of attachment, said carbon atom forming part of one or more non-aromatic ring structures, no carbon-carbon double or triple bonds, and no atoms other than carbon and hydrogen. Non-limiting examples include: —CH(CH₂)₂ (cyclopropyl), cyclobutyl, cyclopentyl, or cyclohexyl (Cy). As used herein, the term does not preclude the presence of one or more alkyl groups (carbon number limitation permitting) attached to a carbon atom of the non-aromatic ring structure. The term “cycloalkanediyl” refers to a divalent saturated aliphatic group with two carbon atoms as points of attachment, no carbon-carbon double or triple bonds, and no atoms other than carbon and hydrogen. The group

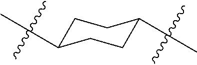 [SMILES: *C1CCC(*)CC1]

is a non-limiting example of cycloalkanediyl group. A “cycloalkane” refers to the class of compounds having the formula H-R, wherein R is cycloalkyl as this term is defined above.

The term “alkenyl” refers to a monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” refers to a divalent unsaturated aliphatic group, with two carbon atoms as points of attachment, a linear or branched acyclic structure, at least one nonaromatic carbon-carbon double bond, no carbon-carbon triple bonds, and no atoms other than carbon and hydrogen. The groups —CH═CH—, —CH═C(CH₃)CH₂—, —CH═CHCH₂—, and —CH₂CH═CHCH₂— are non-limiting examples of alkenediyl groups. It is noted that while the alkenediyl group is aliphatic, once connected at both ends, this group is not precluded from forming part of an aromatic structure. The terms “alkene” and “olefin” are synonymous and refer to the class of compounds having the formula H-R, wherein R is alkenyl as this term is defined above. Similarly, the terms “terminal alkene” and “α-olefin” are synonymous and refer to an alkene having just one carbon-carbon double bond, wherein that bond is part of a vinyl group at an end of the molecule.

The term “alkynyl” refers to a monovalent unsaturated aliphatic group with a carbon atom as the point of attachment, a linear or branched acyclic structure, at least one carbon-carbon triple bond, and no atoms other than carbon and hydrogen. As used herein, the term alkynyl does not preclude the presence of one or more non-aromatic carbon-carbon double bonds. The groups —C≡CH, —C≡CCH₃, and —CH₂C≡CCH₃ are non-limiting examples of alkynyl groups. The term “alkynediyl” refers to a divalent unsaturated aliphatic group, with two carbon atoms as points of attachment, a linear or branched acyclic structure, at least one nonaromatic carbon-carbon triple bond, no carbon-carbon double bonds, and no atoms other than carbon and hydrogen. The groups —C≡C—, —C≡CCH₂—, and —CH₂C═CCH₂— are non-limiting examples of alkynediyl groups. It is noted that while the alkynediyl group is aliphatic, once connected at both ends, this group is not precluded from forming part of an aromatic structure. An “alkyne” refers to the class of compounds having the formula H-R, wherein R is alkynyl.

The term “aryl” refers to a monovalent unsaturated aromatic group with an aromatic carbon atom as the point of attachment, said carbon atom forming part of a one or more aromatic ring structures, each with six ring atoms that are all carbon, and wherein the group consists of no atoms other than carbon and hydrogen. If more than one ring is present, the rings may be fused or unfused. Unfused rings are connected with a covalent bond. As used herein, the term aryl does not preclude the presence of one or more alkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl (e.g., 4-phenylphenyl). The term “arenediyl” refers to a divalent aromatic group with two aromatic carbon atoms as points of attachment, said carbon atoms forming part of one or more six-membered aromatic ring structures, each with six ring atoms that are all carbon, and wherein the divalent group consists of no atoms other than carbon and hydrogen. As used herein, the term arenediyl does not preclude the presence of one or more alkyl groups (carbon number limitation permitting) attached to the first aromatic ring or any additional aromatic ring present. If more than one ring is present, the rings may be fused or unfused. Unfused rings are connected with a covalent bond. Non-limiting examples of arenediyl groups include:

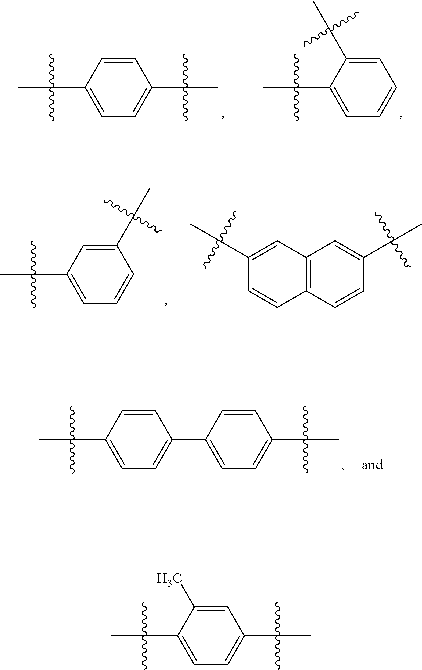

An “arene” refers to the class of compounds having the formula H-R, wherein R is aryl as that term is defined above. Benzene and toluene are non-limiting examples of arenes.

The term “aralkyl” refers to the monovalent group -alkanediyl-aryl, in which the terms alkanediyl and aryl are each used in a manner consistent with the definitions provided above. Non-limiting examples are: phenylmethyl (benzyl, Bn) and 2-phenyl-ethyl.

The term “heteroaryl” refers to a monovalent aromatic group with an aromatic carbon atom or nitrogen atom as the point of attachment, said carbon atom or nitrogen atom forming part of one or more aromatic ring structures, each with three to eight ring atoms, wherein at least one of the ring atoms of the aromatic ring structure(s) is nitrogen, oxygen or sulfur, and wherein the heteroaryl group consists of no atoms other than carbon, hydrogen, aromatic nitrogen, aromatic oxygen and aromatic sulfur. If more than one ring is present, the rings are fused; however, the term heteroaryl does not preclude the presence of one or more alkyl or aryl groups (carbon number limitation permitting) attached to one or more ring atoms. Non-limiting examples of heteroaryl groups include benzoxazolyl, benzimidazolyl, furanyl, imidazolyl (Im), indolyl, indazolyl (Im), isoxazolyl, methylpyridinyl, oxazolyl, oxadiazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. A “heteroarene” refers to the class of compounds having the formula H-R, wherein R is heteroaryl. Pyridine and quinoline are non-limiting examples of heteroarenes.

The term “heterocycloalkyl” refers to a monovalent non-aromatic group with a carbon atom or nitrogen atom as the point of attachment, said carbon atom or nitrogen atom forming part of one or more non-aromatic ring structures, each with three to eight ring atoms, wherein at least one of the ring atoms of the non-aromatic ring structure(s) is nitrogen, oxygen or sulfur, and wherein the heterocycloalkyl group consists of no atoms other than carbon, hydrogen, nitrogen, oxygen and sulfur. If more than one ring is present, the rings are fused. As used herein, the term does not preclude the presence of one or more alkyl groups (carbon number limitation permitting) attached to one or more ring atoms. Also, the term does not preclude the presence of one or more double bonds in the ring or ring system, provided that the resulting group remains non-aromatic. Non-limiting examples of heterocycloalkyl groups include aziridinyl, azetidinyl, pyrrolidinyl, piperidinyl, piperazinyl, morpholinyl, thiomorpholinyl, tetrahydrofuranyl, tetrahydrothiofuranyl, tetrahydropyranyl, pyranyl, oxiranyl, and oxetanyl. The term “N-heterocycloalkyl” refers to a heterocycloalkyl group with a nitrogen atom as the point of attachment. N-pyrrolidinyl is an example of such a group.

The term “acyl” refers to the group —C(O)R, in which R is a hydrogen, alkyl, cycloalkyl, or aryl as those terms are defined above. The groups, —CHO, —C(O)CH₃ (acetyl, Ac), —C(O)CH₂CH₃, —C(O)CH(CH₃)₂, —C(O)CH(CH₂)₂, —C(O)C₆H₅, and —C(O)C₆H₄CH₃ are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkyl group, as defined above, attached to a —CHO group.

The term “alkoxy” refers to the group —OR, in which R is an alkyl, as that term is defined above. Non-limiting examples include: —OCH₃ (methoxy), —OCH₂CH₃ (ethoxy), —OCH₂CH₂CH₃, —OCH(CH₃)₂ (isopropoxy), or —OC(CH₃)₃ (tert-butoxy). The terms “cycloalkoxy”, “alkenyloxy”, “alkynyloxy”, “aryloxy”, “aralkoxy”, “heteroaryloxy”, “heterocycloalkoxy”, and “acyloxy”, when used without the “substituted” modifier, refers to groups, defined as —OR, in which R is cycloalkyl, alkenyl, alkynyl, aryl, aralkyl, heteroaryl, heterocycloalkyl, and acyl, respectively. The term “alkylthio” and “acylthio” refers to the group —SR, in which R is an alkyl and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group.

The term “alkylamino” refers to the group —NHR, in which R is an alkyl, as that term is defined above. Non-limiting examples include: —NHCH₃ and —NHCH₂CH₃. The term “dialkylamino” refers to the group —NRR′, in which R and R′ can be the same or different alkyl groups. Non-limiting examples of dialkylamino groups include: —N(CH₃)₂ and —N(CH₃)(CH₂CH₃). The terms “cycloalkylamino”, “alkenylamino”, “alkynylamino”, “arylamino”, “aralkylamino”, “heteroarylamino”, “heterocycloalkylamino”, and “alkoxyamino” when used without the “substituted” modifier, refers to groups, defined as —NHR, in which R is cycloalkyl, alkenyl, alkynyl, aryl, aralkyl, heteroaryl, heterocycloalkyl, and alkoxy, respectively. A non-limiting example of an arylamino group is —NHC₆H₅. The terms “dicycloalkylamino”, “dialkenylamino”, “dialkynylamino”, “diarylamino”, “diaralkylamino”, “diheteroarylamino”, “diheterocycloalkylamino”, and “dialkoxyamino”, refers to groups, defined as —NRR′, in which R and R′ are both cycloalkyl, alkenyl, alkynyl, aryl, aralkyl, heteroaryl, heterocycloalkyl, and alkoxy, respectively. Similarly, the term alkyl(cycloalkyl)amino refers to a group defined as —NRR′, in which R is alkyl and R′ is cycloalkyl. The term “amido” (acylamino), when used without the “substituted” modifier, refers to the group —NHR, in which R is acyl, as that term is defined above. A non-limiting example of an amido group is —NHC(O)CH₃.

An “amine protecting group” or “amino protecting group” is well understood in the art. An amine protecting group is a group which prevents the reactivity of the amine group during a reaction which modifies some other portion of the molecule and can be easily removed to generate the desired amine. Amine protecting groups can be found at least in Greene and Wuts, 1999, which is incorporated herein by reference. Some non-limiting examples of amino protecting groups include formyl, acetyl, propionyl, pivaloyl, t-butylacetyl, 2-chloroacetyl, 2-bromoacetyl, trifluoroacetyl, trichloroacetyl, o-nitrophenoxyacetyl, α-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like; alkoxy- or aryloxycarbonyl groups (which form urethanes with the protected amine) such as benzyloxycarbonyl (Cbz), p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxy-benzyloxycarbonyl, 1-(p-biphenylyl)-1-methylethoxycarbonyl, α,α-dimethyl-3,5-dimethoxybenzyloxycarbonyl, benzhydryloxycarbonyl, t-butyloxycarbonyl (Boc), diisopropylmethoxycarbonyl, isopropyloxycarbonyl, ethoxycarbonyl, methoxycarbonyl, allyloxycarbonyl (Alloc), 2,2,2-trichloroethoxycarbonyl, 2-trimethylsilylethyloxycarbonyl (Teoc), phenoxycarbonyl, 4-nitrophenoxycarbonyl, fluorenyl-9-methoxycarbonyl (Fmoc), cyclopentyloxycarbonyl, adamantyloxycarbonyl, cyclohexyloxycarbonyl, phenylthiocarbonyl and the like; aralkyl groups such as benzyl, triphenylmethyl, benzyloxymethyl and the like; and silyl groups such as trimethylsilyl and the like. Additionally, the “amine protecting group” can be a divalent protecting group such that both hydrogen atoms on a primary amine are replaced with a single protecting group. In such a situation the amine protecting group can be phthalimide (phth) or a substituted derivative thereof wherein the term “substituted” is as defined above. In some embodiments, the halogenated phthalimide derivative may be tetrachlorophthalimide (TCphth). When used herein, a “protected amino group”, is a group of the formula PG_{MA}NH— or PG_{DA}N— wherein PG_{MA} is a monovalent amine protecting group, which may also be described as a “monvalently protected amino group” and PG_{DA} is a divalent amine protecting group as described above, which may also be described as a “divalently protected amino group”.

When a chemical group is used with the “substituted” modifier, one or more hydrogen atom has been replaced, independently at each instance, by —OH, —F, —Cl, —Br, —I, —NH₂, —NO₂, —CO₂H, —CO₂CH₃, —CO₂CH₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. For example, the following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups. Non-limiting examples of substituted aralkyls are: (3-chlorophenyl)-methyl, and 2-chloro-2-phenyl-eth-1-yl. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups. The groups —NHC(O)OCH₃ and —NHC(O)NHCH₃ are non-limiting examples of substituted amido groups.

As used herein, the term “functional group” refers to any chemical group or substituent covalently bound to a core structure. Functional groups may include but are not limited to hydrogen, alkyl, alkenyl, alkynyl, cycloalkyl, heterocycloalkyl, aryl, heteroaryl, aralkyl, —OH, —F, —Cl, —Br, —I, —NH₂, —NO₂, —CO₂H, —CO₂CH₃, —CO₂CH₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, —S(O)₂NH₂ or a combination or substituted version of any of these groups.

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects or patients.

An “active ingredient” (AI) or active pharmaceutical ingredient (API) (also referred to as an active compound, active substance, active agent, pharmaceutical agent, agent, biologically active molecule, or a therapeutic compound) is the ingredient in a pharmaceutical drug that is biologically active.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to a subject or patient for treating or preventing a disease, is an amount sufficient to effect such treatment or prevention of the disease.

An “excipient” is a pharmaceutically acceptable substance formulated along with the active ingredient(s) of a medication, pharmaceutical composition, formulation, or drug delivery system. Excipients may be used, for example, to stabilize the composition, to bulk up the composition (thus often referred to as “bulking agents,” “fillers,” or “diluents” when used for this purpose), or to confer a therapeutic enhancement on the active ingredient in the final dosage form, such as facilitating drug absorption, reducing viscosity, or enhancing solubility. Excipients include pharmaceutically acceptable versions of antiadherents, binders, coatings, colors, disintegrants, flavors, glidants, lubricants, preservatives, sorbents, sweeteners, and vehicles. The main excipient that serves as a medium for conveying the active ingredient is usually called the vehicle. Excipients may also be used in the manufacturing process, for example, to aid in the handling of the active substance, such as by facilitating powder flowability or non-stick properties, in addition to aiding in vitro stability such as prevention of denaturation or aggregation over the expected shelf life. The suitability of an excipient will typically vary depending on the route of administration, the dosage form, the active ingredient, as well as other factors.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e. an enzyme, cell, cell receptor or microorganism) by half.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human patients are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds disclosed herein which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this disclosure is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

A “pharmaceutically acceptable carrier,” “drug carrier,” or simply “carrier” is a pharmaceutically acceptable substance formulated along with the active ingredient medication that is involved in carrying, delivering and/or transporting a chemical agent. Drug carriers may be used to improve the delivery and the effectiveness of drugs, including for example, controlled-release technology to modulate drug bioavailability, decrease drug metabolism, and/or reduce drug toxicity. Some drug carriers may increase the effectiveness of drug delivery to the specific target sites. Examples of carriers include: liposomes, microspheres (e.g., made of poly(lactic-co-glycolic) acid), albumin microspheres, synthetic polymers, nanofibers, protein-DNA complexes, protein conjugates, erythrocytes, virosomes, and dendrimers.

A “pharmaceutical drug” (also referred to as a pharmaceutical, pharmaceutical preparation, pharmaceutical composition, pharmaceutical formulation, pharmaceutical product, medicinal product, medicine, medication, medicament, or simply a drug, agent, or preparation) is a composition used to diagnose, cure, treat, or prevent disease, which comprises an active pharmaceutical ingredient (API) (defined above) and optionally contains one or more inactive ingredients, which are also referred to as excipients (defined above).

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2ⁿ, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease or symptom thereof in a subject or patient that is experiencing or displaying the pathology or symptomatology of the disease.

The term “unit dose” refers to a formulation of the compound or composition such that the formulation is prepared in a manner sufficient to provide a single therapeutically effective dose of the active ingredient to a patient in a single administration. Such unit dose formulations that may be used include but are not limited to a single tablet, capsule, or other oral formulations, or a single vial with a syringeable liquid or other injectable formulations.

The above definitions supersede any conflicting definition in any reference that is incorporated by reference herein. The fact that certain terms are defined, however, should not be considered as indicative that any term that is undefined is indefinite. Rather, all terms used are believed to describe the disclosure in terms such that one of ordinary skill can appreciate the scope and practice the present disclosure.

### V. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Synthetic Methodology

### A. Synthesis of 4-(5-hydroxy-2-oxobenzo[d][1,3]oxathiol-7-yl)benzenaminium

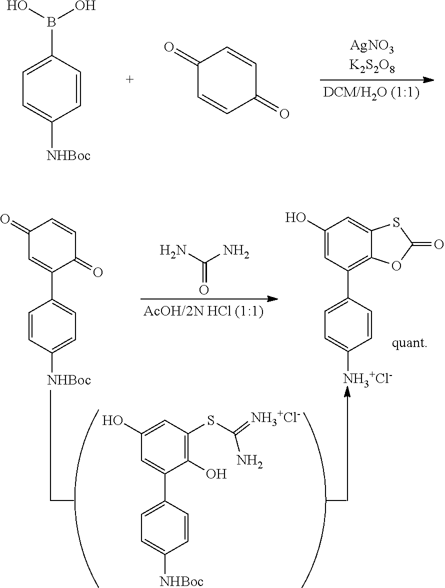

### B. Synthesis of CD02 Analogues

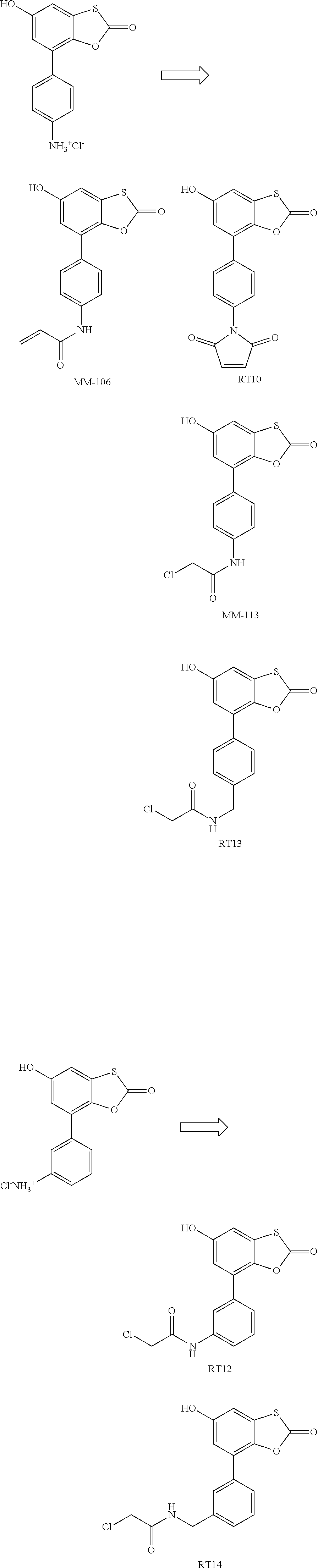

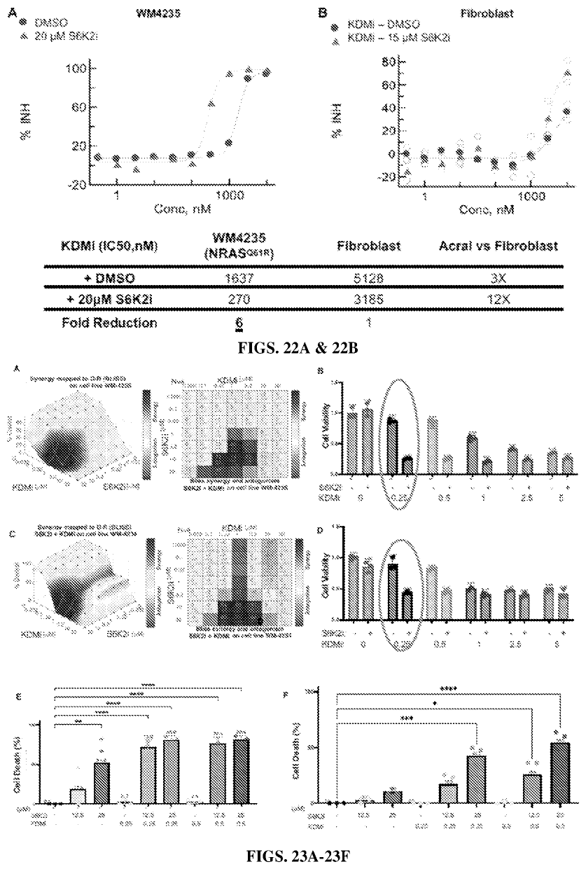

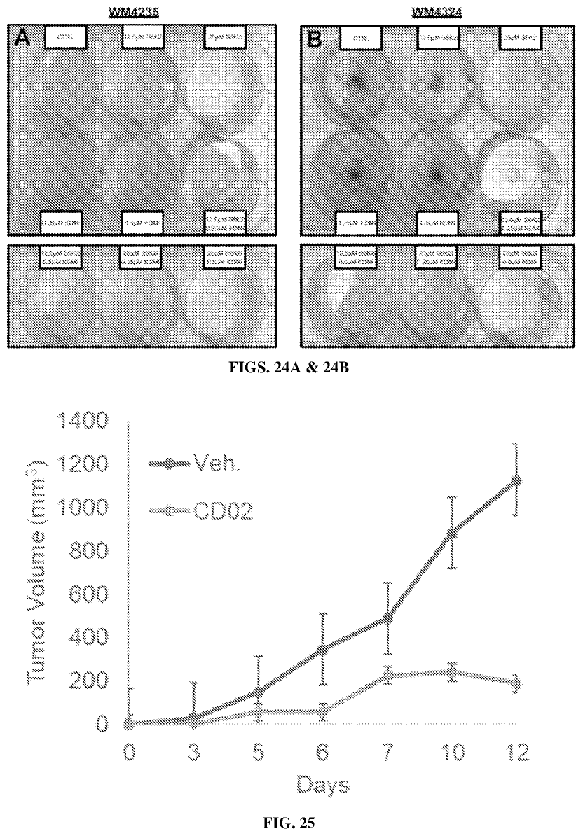

Similarly, the compounds herein maybe used to treat cancers such as acral melanoma. In particular, the treatment of acral melanoma may be enhanced through the use of a KDM inhibitor. See, FIGS. 21-25.

### Example 2—S6K2 is a Therapeutic Target for Cancer Such as NRAS Mutant Melanoma

a. S6K2 is a Vulnerability in NRAS-Mutant Melanoma Resistant to MAPK Inhibitors

Since MAPK inhibition elicits heterogeneous effects in NRAS-mutant melanoma (Ascierto et al., 2013), we sought to identify potential vulnerabilities in MAPK inhibitor (MAPKi)-resistant tumor cells. The effect of MEK (trametinib and MEK162) or ERK (SCH-984 and BVD-523) inhibitors, collectively referred to as MAPKi, was first analyzed on viability of NRAS-mutant melanoma cells. MAPKi induced variable effects on viability, proliferation, and cell death (FIG. 26A-E), despite effective and persistent inhibition of the MAPK pathway (FIG. 26F). Cells were classified as MAPKi-sensitive (MAPKi-S) based on their response to MAPKi, defined as ≥75% suppression of BrdU incorporation and ≥35% cell death (FIGS. 26D & 26E). Conversely, MAPKi elicited modest or no suppression of BrdU (<75%; FIG. 26D) and marginal induction of cell death (<35%; FIG. 26E) in the MAPKi-resistant (MAPKi-R) cells.

To further explore signaling perturbations in MAPKi-R vs. MAPKi-S cells, cells treated with either the MEK inhibitor trametinib (Tram) or the ERK inhibitor SCH772984 (SCH984) were analyzed by Western blotting and RPPA (FIG. 27A, FIG. 26F-G). MAPKi similarly suppressed the MAPK pathway in both sensitive and resistant cells. MAPKi-treatment did not induce significant differential activation of PI3K/AKT (FIG. 27A). In contrast, MAPKi treatment blocked phosphorylation of S6 kinase (S6K) and its substrate S6 in MAPKi-S, but not in MAPKi-R melanoma cells (FIG. 27A and FIG. 26F).

Since S6K is also a bona fide effector of the PI3K/mTOR pathway, it was further examined the impact of MAPK or PI3K/mTOR signaling on S6K. Constitutively active S6K(15) (S6K1^{T389E} or S6K2^{T388E}) constructs were ectopically expressed in MAPKi-sensitive or resistant cells and assessed levels of pS6^{S240/244}, a site exclusively phosphorylated by S6K(15) (FIG. 27B-C and FIG. 26H-1). Treatment of MAPKi-S cells with MAPKi (SCH984) or PI3K/mTORi (GSK458) decreased phosphorylation of S6; ectopic expression of S6K1^{T389E} or S6K2^{T388E} rescued pS6 levels in cells treated with SCH984 or GSK458 (FIG. 27B-C and FIG. 26I). These data indicate that S6K mediates both MAPK and PI3K/mTOR signaling in MAPKi-S cells. In contrast, treatment of MAPKi-R cells with MAPKi had no effect on phosphorylation of S6, whereas treatment with PI3K/mTORi effectively suppressed pS6 (FIG. 27B-C and FIG. 26I). Ectopic expression of S6K1^{T389E} or S6K2^{T388E} rescued pS6 levels in MAPKi-R cells treated with PI3K/mTORi (FIG. 27B and FIG. 26I). Together, these data indicate that S6K activity is regulated by the PI3K/mTOR pathway in MAPKi-R cells. Moreover, these findings highlight that different upstream pathways differentially impinge on S6 kinase in MAPKi-R vs. MAPKi-S cells (FIG. 27D).

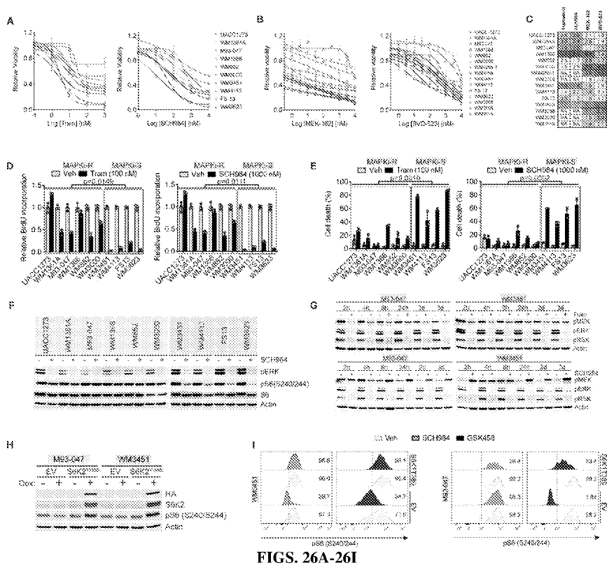

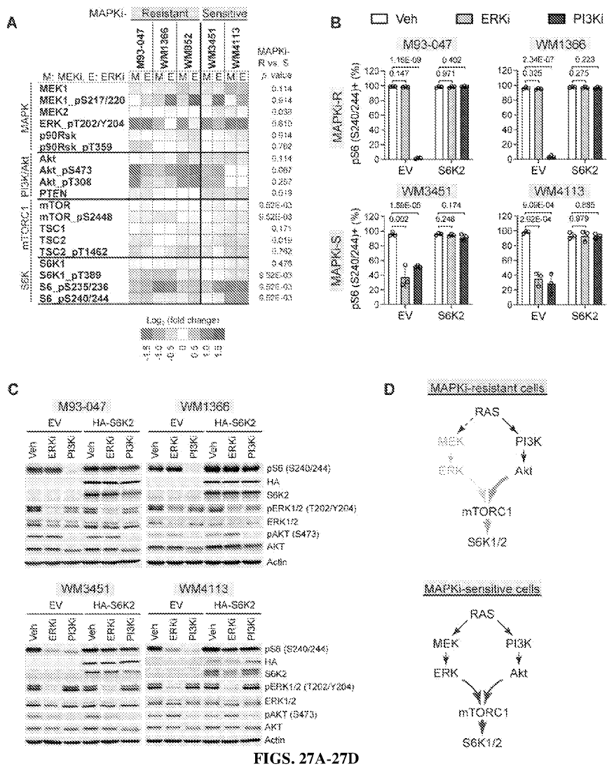

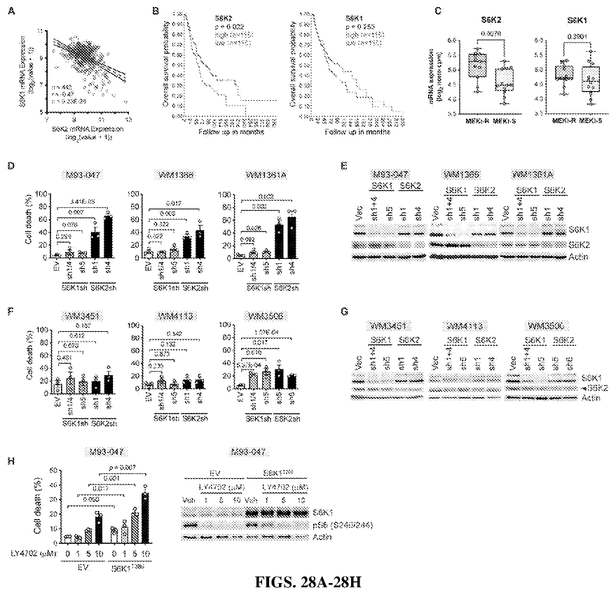

The expression of S6K1/2 in patient samples was next examined by interrogating the TCGA skin cutaneous melanoma dataset. It was noted that S6K2 mRNA levels were inversely correlated with S6K1 (n=443, R=−0.47, p val=9.23 e-26; FIG. 28A). It was also noted that in melanoma patients, high expression of S6K2 (but not S6K1) was associated with poor survival (FIG. 28B). Furthermore, analysis of RNA-seq data from NRAS^{mut} melanoma patient-derived cells (Nagler et al., 2020) (FIG. 28C) revealed that S6K2 mRNA levels are significantly higher in MEKi-R tumor cells than in MEKi-S tumor cells. S6K1 mRNA levels were not significantly different in MEKi-S vs. MEKi-R-patient derived tumor cells. These data support the notion that S6K2 could have an important role in melanoma and MAPKi resistance, and thereby constitutes a vulnerability in this tumor type.

To assess the role of S6K in NRAS-mutant melanoma cells, S6K1 or S6K2 were silenced in 6 NRAS-mutant melanoma lines (3 MAPKi-S and 3 MAPKi-R) using multiple hairpins. Depletion of S6K2, but not S6K1, induced cell death in MAPKi-R cells (FIG. 28D, E). Notably, neither depletion of S6K1 nor S6K2 triggered substantial cell death in MAPKi-S NRAS-mutant melanoma cells (FIG. 28F, G). These data suggest S6K2 is a vulnerability in NRAS-mutant melanoma cells that are resistant to MAPKi. To mimic the effects of an S6K2-specific inhibitor, S6K1 in cells was ectopically expressed activated and treated with the pan-S6K inhibitor LY2584702 (LY4702) (FIG. 28H). Activated S6K1 enhanced LY4702 cytotoxicity, rather than compensating for it, suggesting that sensitivity to pan-S6K inhibition may be mediated via S6K2 inhibition. Collectively, these results indicate that selective inhibition of S6K2, in the context of active S6K1, induces death of MAPKi-R NRAS-mutant melanoma cells.

c. Depletion of S6K2 Triggers Lipid Peroxidation and Facilitates Cell Death

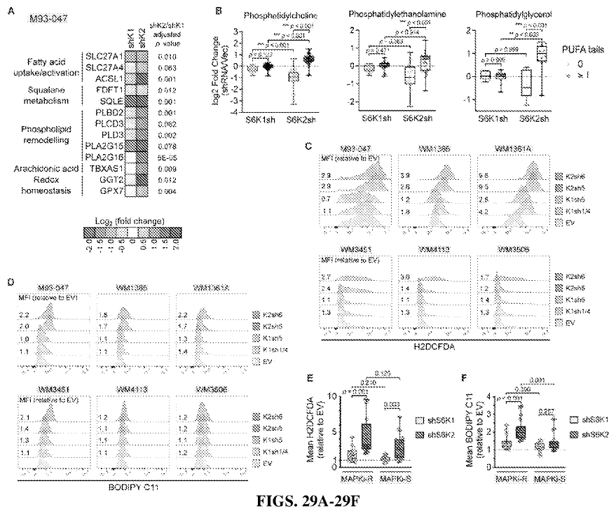

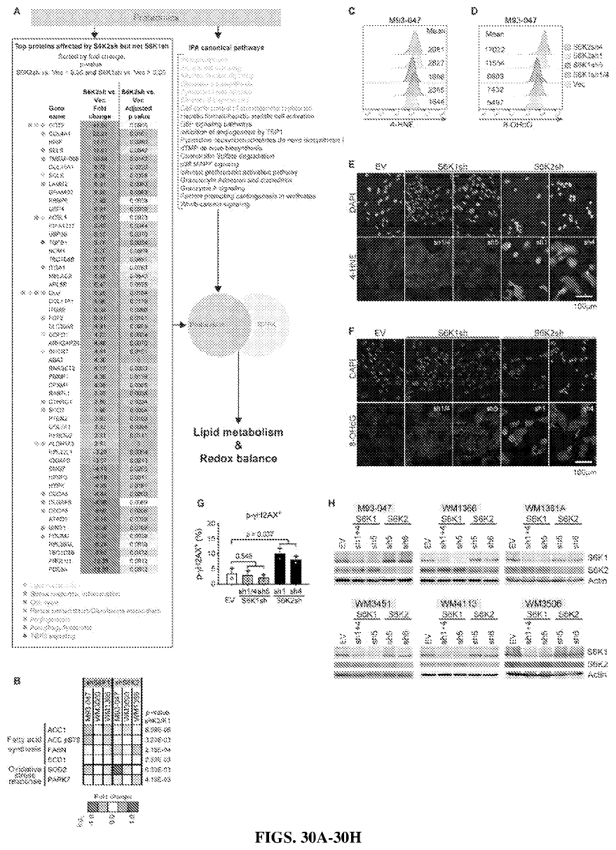

To investigate the mechanism(s) whereby S6K2 depletion triggers cell death in MAPKi-R melanoma cells, the proteome was surveyed to identify proteins that were differentially affected by depletion of S6K2 vs. S6K1 in MAPKi-R melanoma cells. Depletion of S6K2 was coupled to enhanced expression of proteins involved in lipid synthesis, fatty acid synthesis, uptake and activation, and phospholipid remodeling (FIG. 29A and FIG. 30A). To further assess if lipid metabolism was perturbed by S6K2 depletion, unbiased global lipidomic analysis in S6K2 or S6K1-depleted cells was performed. Notably, depletion of S6K2 led to an enrichment of specific lipid species containing polyunsaturated fatty acyl chains (PUFAs) such as phosphatidylcholine, phosphatidylethanolamine and phosphatidylglycerol (FIG. 29B), consistent with disrupted lipid homeostasis. These results suggest that S6K2 blockade is coupled to an enhanced degree of fatty acid unsaturation in some lipid classes.

It was also noted that depletion of S6K1 or S6K2 led to increased levels of proteins involved in the oxidative stress response, including ROS sensing/detoxifying enzymes such as GGT2, SOD2 and PARK7 (FIG. 29A and FIG. 30A-B). While depletion of either S6K1 or S6K2 (FIG. 30H) led to increased ROS levels (FIG. 29C, E), depletion of S6K2 induced significantly higher relative ROS levels compared to S6K1 depletion in both MAPKi-R cells and MAPKi-S cells (FIG. 29C, E). Although depletion of S6K2 increased ROS levels in MAPKi-S cells, these cells did not undergo cell death (FIG. 28F), implying that ROS alone are not sufficient to induce cell death or that MAPKi-S cells are able to counteract redox imbalance. The perturbation of redox and lipid homeostasis upon S6K2 knockdown raised the possibility that S6K2 depletion could be triggering lipid peroxidation (Ayala et al., 2014). Indeed, depletion of S6K2 (but not S6K1) substantially enhanced lipid peroxidation and oxidative stress, as indicated by the lipid ROS sensor Bodipy-C11 (FIG. 29D, F), generation of 4-HNE adducts (FIG. 30C, E), and increased levels of nucleic acid oxidative damage (8-OHdG; FIG. 30D, F) in MAPKi-R cells. Whereas general S6K2 depletion induced similar increase in general ROS levels in both MAPKi-S and MAPKi-R cells, lipid ROS were preferentially induced in MAPKi-R cells (FIG. 29C-F). Together, these results support the premise that depletion of S6K2 enhances lipid metabolism and ROS levels, which jointly trigger lethal lipid peroxidation and oxidative cell death in MAPKi-R NRAS-mutant melanoma.

d. S6K2 Depletion Activates a Terminal Unfolded Protein Response (UPR) with Concomitant Upregulation of PPARα

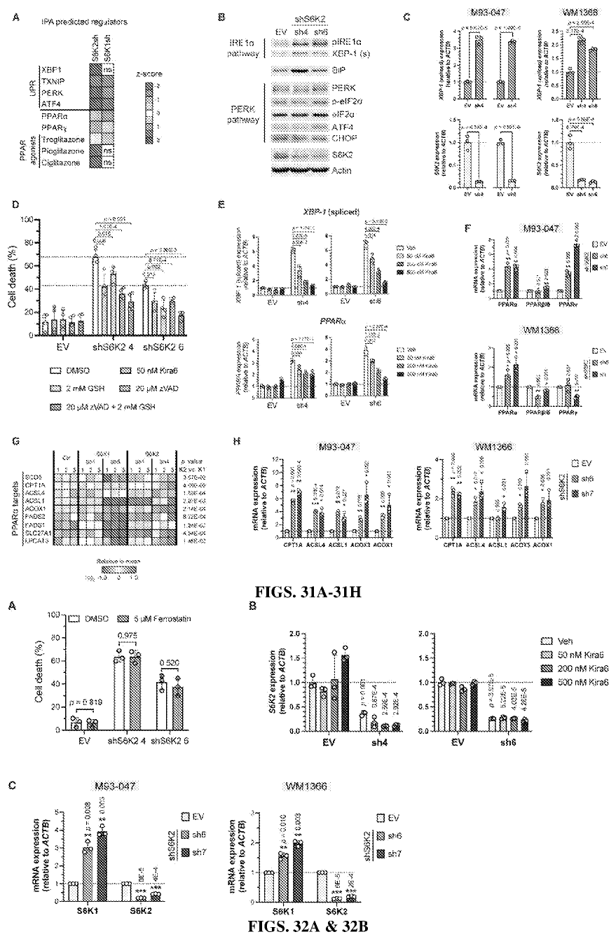

Excessive lipid peroxidation has been linked to different types of cell death including apoptosis and ferroptosis (Ayala et al., 2014; Magtanog et al., 2016; Dixon et al., 2012; Yang et al., 2014; Viswanathan et al., 2017; Shimada et al., 2016; Friedmann Angeli et al., 2014). To identify S6K2 effectors that could trigger cytotoxicity, global transcriptional changes induced by S6K2 depletion were assessed. Transcriptomic analysis indicated a general enrichment of unfolded protein response (UPR) regulators in both S6K1- and S6K2-depleted cells, whereas XBP-1 and target genes of the lipid metabolism regulator PPAR (Kersten, 2014; Rakhshandehroo et al., 2010) were selectively increased in S6K2-depleted cells (FIG. 31A, G). Oxidative or metabolic stress can disrupt endoplasmic reticulum (ER) homeostasis, promoting the aberrant accumulation of misfolded proteins, inducing ER stress and further dysfunction of lipid metabolism (Han and Kaufman, 2016). ER stress initiates the unfolded protein response (UPR) through BiP-mediated activation of three canonical pathways: IRE1-XBP-1, PERK-CHOP and ATF6 (Almanza et al., 2019), either restoring ER homeostasis under conditions of mild ER stress or triggering apoptosis when ER stress is severe and persistent. Increased levels of activated, spliced XBP-1 were found and its regulator phospho-IRE1α in S6K2-depleted cells (FIG. 31A-C). In contrast, S6K2 depletion had a negligible effect on PERK-CHOP signaling (FIG. 31B). Treatment of MAPKi-R NRAS-mutant melanoma cells with the IRE1α inhibitor Kira6 partially attenuated cell death induced by S6K2 depletion, indicating that the cytotoxic effects of S6K2 depletion are partially mediated by a terminal UPR that activates apoptosis. Additionally, treatment with the pan-caspase inhibitor zVAD or the antioxidant glutathione (GSH) also attenuated cell death (FIG. 31D). In contrast, treatment of MAPKi-R NRAS-mutant melanoma cells with the ferroptosis inhibitor, ferrostatin-1, did not attenuate cell death triggered by S6K2 depletion (FIG. 32A). Upregulation of XBP-1 splicing and PPARα expression caused by S6K2 knockdown was suppressed by IRE1α inhibition with Kira6 in a dose-dependent manner, suggesting that PPARα could be a key effector downstream of ER stress (FIG. 31E, FIG. 32B). Since XBP-1 directly binds to and activates PPARα and PPARγ (Sengupta et al., 2013; Kim et al., 2012), the expression of the PPAR gene family were assessed following S6K2 knockdown (FIG. 31F, FIG. 32C). PPARα expression was consistently elevated by S6K2 depletion in MAPKi-R cells, whereas PPARγ was upregulated in a single cell line. Further, S6K2 depletion led to upregulation of representative PPAR targets involved in lipid metabolism in both MAPKi-R cell lines (FIG. 31G-H). Together, these data suggest that S6K2 depletion causes ER stress and that the IRE1α-XBP-1-PPARα signaling network contributes to cell death and lipid imbalance triggered by S6K2 depletion.

e. S6K2 Negatively Regulates PPARα

It was next sought to define the mechanism whereby S6K2 could be regulating PPARα. The transcriptional activity of PPARα is inhibited by the nuclear receptor corepressor NCoR1 (Sengupta et al., 2013), and S6K2 has been implicated in this regulation (Kim et al., 2012). Hence, whether S6K2 could regulate this interaction in NRAS-mutant melanoma cells was examined using the technique of proximity ligation assays (PLA). PLA supported the existence of a complex between S6K2, PPARα and its co-repressor NCoR1 in MAPKi-R cells (FIG. 33A-C). S6K2 depletion markedly diminished the interaction of NCoR1 with PPARα in MAPKi-R cells (FIG. 33D, F, FIG. 34A), indicating that S6K2 blockade is coupled to PPARα activation. In comparison, a weak and diffuse PPARα-NCOR1 interaction was noted in MAPKi-S cells, which was slightly enhanced following S6K2 depletion (FIG. 33E, F, FIG. 34B). Together, these data support a model whereby S6K2 regulates the interaction between of NCoR1 and PPARα; S6K2 depletion disrupts this complex leading to activation of the PPARα axis, thereby facilitating transcription of genes encoding proteins involved in lipid peroxidation and oxidative cell death in MAPKi-R NRAS-mutant melanoma.

f. PUFAs Potentiate the Oxidative Cell Death Inducing Effect of PPARα Agonists

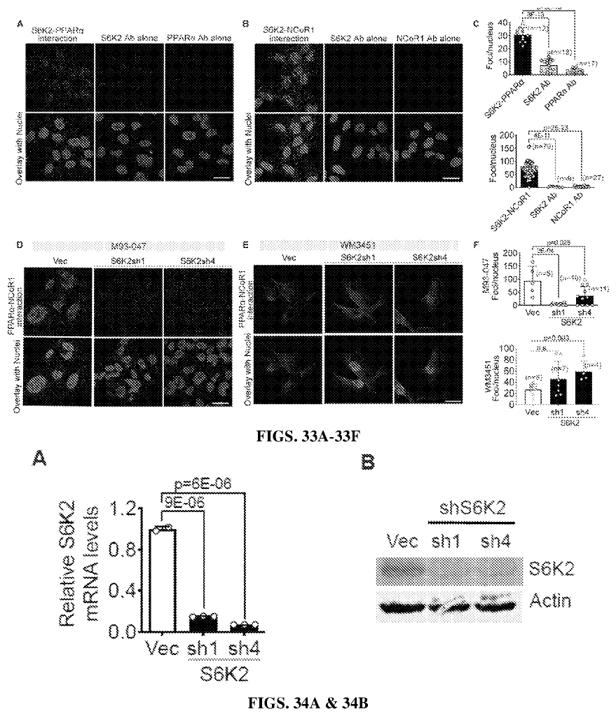

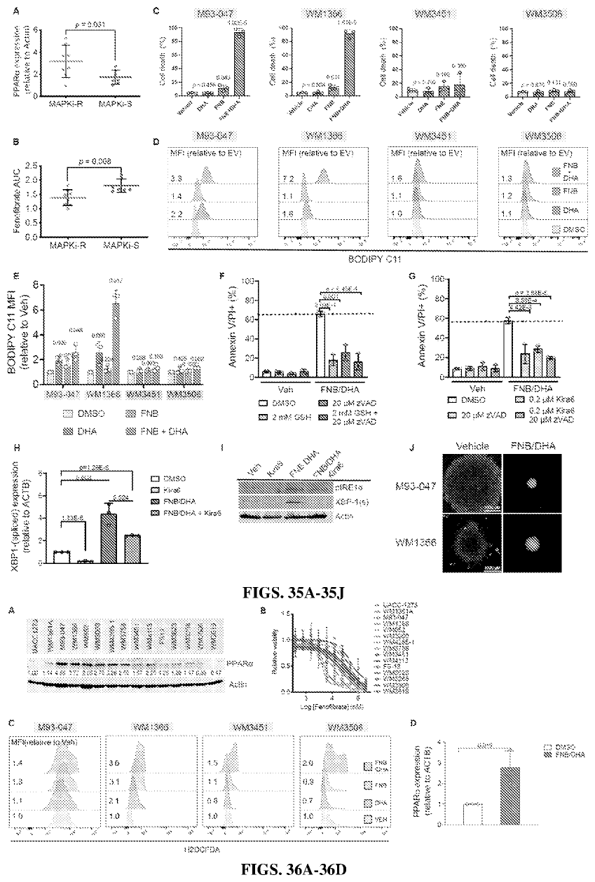

Based on these data indicating that PPARα facilitates lethal lipid peroxidation, it was next asked if PPARα agonists could induce anti-melanoma effects. It was noted that PPARα protein levels were higher in MAPKi-R NRAS-mutant melanoma cells (FIG. 35A and FIG. 36A) and that MAPKi-R cells were more sensitive to the PPARα agonist fenofibrate (FNB) than MAPKi-S/PPARα-low cells (FIG. 35B and FIG. 36B). FNB is a weak PPARα agonist, which can decrease GSH levels and has been shown to modify lipid and lipoprotein composition and metabolism by a variety of mechanisms (Lian et al., 2018). Treatment with FNB alone did not induce cell death in MAPKi-R cells (FIG. 35C). Since these lipidomic data revealed S6K2 depletion induces accumulation of PUFAs (FIG. 29B), it was wondered whether addition of docosahexaenoic acid (DHA) as a source of PUFAs could potentiate the effect of FNB in inducing lethal lipid peroxidation and cell death. To this end, MAPKi-R and MAPKi-S cells were treated with FNB+/−DHA. While neither FNB nor DHA as single agents induced significant cell death, the combination of FNB+DHA triggered significant cell death in MAPKi-R cells, but not in MAPKi-S NRAS-mutant melanoma cells (FIG. 35C). Similar to S6K2 depletion, the FNB/DHA combination induced ROS levels (FIG. 36C). Consistent with the effects of S6K2 depletion, treatment with FNB+DHA induced lipid peroxidation selectively in MAPKi-R cells (FIG. 35D, E). Further supporting the notion that the FNB/DHA combination phenocopies S6K2 depletion, the antioxidant GSH, pan-caspase inhibitor, zVAD and IRE1α inhibitor, Kira6 attenuated FNB/DHA-induced cell death (FIG. 35F, G). Additionally, the FNB/DHA combination induced both spliced XBP-1 and PPARα (FIG. 36D, FIG. 35H, I), and cotreatment with Kira6 suppressed the activation of XBP-1. Moreover, the FNB/DHA combination induced cell death in MAPKi-R 3D melanoma spheroids (FIG. 35J). Taken together, these results indicate that MAPKi-R melanoma cells are sensitive to pharmacological activation of PPARα in combination with enhanced fatty acid desaturation and lipid peroxidation.

g. MAPKi-Resistant NRAS-Mutant Melanoma is Sensitive to the Combination of PPARα Agonists and PUFAs

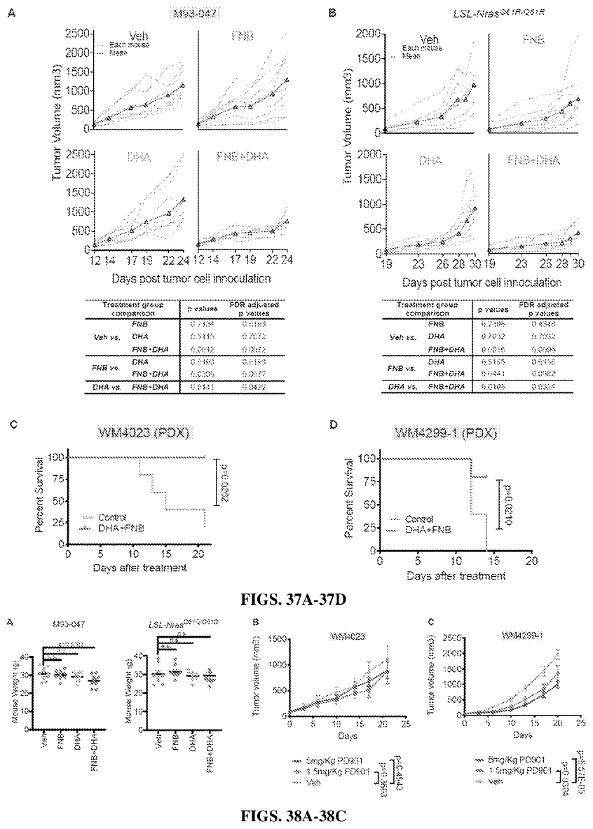

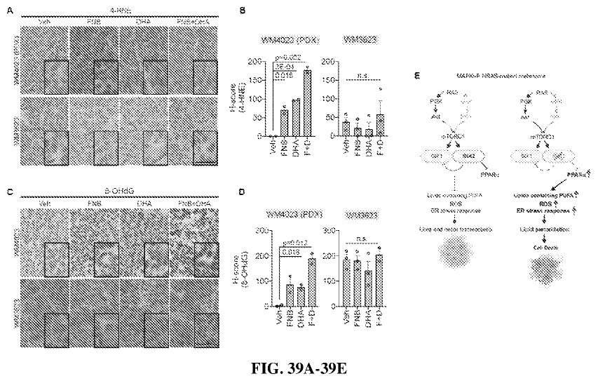

It was next evaluated the efficacy of combining PPARα agonists and DHA in vivo. Combining FNB with DHA suppressed the growth of established NRAS-mutant MAPKi-R xenografts (FIG. 37A) and syngeneic tumors (FIG. 37B) (Burd et al., 2014; Echevarria-Vargas et al., 2018) with no appreciable toxicity (FIG. 38A). Furthermore, FNB+DHA significantly increased the survival of mice bearing MAPKi-R PDXs (FIG. 37C, D, FIG. 38B). Importantly, FNB+DHA induced substantial lipid peroxidation and oxidative damage in MAPKi-R xenografts and PDXs (FIG. 39A-D). These results provide proof-of-principle that combining PPARα agonists with PUFAs facilitates lipid peroxidation, elicits anti-tumor activity, and restrains MAPKi-R NRAS-mutant melanoma. By selectively blocking S6K2 or harnessing the S6K2 subnetwork, it was have identified a strategy that could be potentially exploited as an anti-tumor approach in NRAS-mutant melanoma resistant to MAPKi.

### III. DISCUSSION

Despite significant advances in treating melanoma, effective therapies for NRAS-mutant tumors are sorely needed. One approach to inhibiting NRAS that has been investigated is targeting its proximal downstream effectors, mainly the MAPK and PI3K pathways. While inhibiting either pathway alone is barely effective in NRAS-mutant tumors, inhibiting both pathways leads to unacceptable toxicities in patients. Without wishing to be bound by any theory, it is hypothesized that identifying and inhibiting convergent subnetworks, within the MAPK and PI3K super-networks, could spare the toxicities while triggering death of cancer cells. Then, therefore, a deep analysis of the signaling networks was in MAPKi-resistant NRAS-mutant melanoma. By comparing the effects of MAPK inhibition in MAPKi-S vs. MAPKi-R NRAS-mutant melanoma, a critical subnetwork downstream from the convergence of PI3K and MAPK was mapped. A S6 kinase 1/2 (S6K1/2)-PPARα subnetwork was found that the subnetwork is vital for cancer lipid metabolism. It was uncovered that S6K1/2 is regulated by both the MAPK and PI3K pathways in MAPKi-S cells; in contrast, S6K1/2 is regulated by PI3K in MAPKi-R NRAS-mutant melanoma. Further, uncoupling S6K1 and S6K2 was shown, by selective S6K2 depletion, triggers a lipid metabolic imbalance featuring ER stress and lipid peroxidation, which leads to cell death preferentially in MAPKi-R NRAS-mutant melanomas (FIG. 39E). Both the IRE1α-XBP-1 and PERK-CHOP pathways are implicated in initiating ER stress-induced apoptosis (Szegezdi et al., 2006). Upon S6K2 knockdown, ER stress predominantly activated the IRE1α-XBP-1 pathway. Moreover, cell death was suppressed by the IRE1α inhibitor Kira6, indicating that IRE1α-XBP-1 signaling plays a key role in mediating cell death elicited by S6K2 suppression. These findings underscore that MAPKi-resistant and -sensitive cells have different signaling dependencies both upstream and downstream of mTORC1 and illustrate specialized functions of S6K isoforms, highlighting the biological importance of isoform differentiation. Moreover, the identified subnetwork provides a strategy to pharmacologically induce lethal lipid peroxidation in vivo, which has been limited by the lack of compounds with sufficient bioavailability (Viswandathan et al., 2017; Hangauer et al., 2017; Tsoi et al., 2018). Of note, S6K2 KO mice are viable and develop normally (Pende et al., 2004), indicating that S6K2 is not an essential gene. Collectively, these data suggest that selective targeting of S6K2 or its effectors represents a viable opportunity for therapeutic intervention in S6K2-dependent tumors such as MAPKi-R NRAS-mutant melanoma.

These study establishes that selectively depleting S6K2 induces ER stress and lethal lipid peroxidation in MAPKi-R NRAS-mutant melanomas. This suggests that selective inhibition of S6K2 could curb melanoma through a mechanism unachievable with PI3K or mTOR inhibitors, which inhibit both S6K1 and S6K2. However, selective S6K2 inhibition has not been explored in pre-clinical melanoma models and only one S6K2-selective chemical probe has been reported(50), in part due to the high sequence similarity of S6K2 with S6K1 and the lack of S6K2 crystal structure (Pardo et al., 2013; Karlsson et al., 2015).

The combination of FNB (PPARα agonist) plus DHA (PUFA) recapitulated the activation of ER stress and XBP-1-PPARα signaling observed with genetic depletion of S6K2, promoting lipid peroxidation and cell death. Importantly, both FNB and DHA are compounds that are used in humans with manageable toxicity profiles and, therefore, could be incorporated into new treatments for melanoma. Of note, MAPKi-R and MAPKi-S NRAS-mutant melanomas exhibit different levels of PPARα, which correlate with sensitivity to MAPKi and PPARα agonists. This provides a context in which the proposed approach would be most effective.

Previous studies from our group and others have linked enhanced or persistent mTORC1/S6K1 signaling with acquired resistance to MAPKi in BRAF-mutant melanoma (Atefi et al., 2011; Corcoran et al., 2013; Villanueva et al., 2013) and to MEKi/Cdk4i in NRAS-mutant melanoma (Teh et al., 2018; Romano et al., 2018). Inhibition of the mTORC1/S6K1 axis restrains MAPK-dependent melanomas and tumors with acquired resistance to MAPKi and/or CDK4i. In contrast to strategies relying on mTOR1/S6K1 inhibition, active lipid metabolism was exploited to induce cell death (Green, 2019) by disrupting the S6K1/S6K2/PPARα subnetwork through selective S6K2 blockade. It was noted that while S6K2 blockade led to enhanced expression of proteins involved in lipid synthesis, and accumulation of PUFAS, inhibition of S6K1 minimally perturbed lipid homeostasis. These data support the notion that selective S6K2 inhibition is required to induce lethal peroxidation in melanoma. Inducing severe ER stress and lethal lipid peroxidation provides a therapeutic opportunity to offset drug resistance. Previous studies have shown that MAPKi-R melanomas display elevated levels of oxidative stress and are vulnerable to compounds that enhance ER stress (Cerezo et al., 2016; Wang et al., 2018). Moreover, activation of the UPR can promote anti-tumor immunity in a context-dependent manner (Rufo et al., 2017; Liu et al., 2023). Likewise, tumors characterized by distinctive metabolic states such as those displaying enhanced PUFA biosynthesis are innately vulnerable to lipid peroxidation (Lee et al., 2020; Doll et al., 2017). In sum, these studies establish S6K2 and its effectors as a vulnerability in NRAS-mutant melanomas that are resistant to MAPKi. These results underscore the significance of oncogenic NRAS-induced metabolic dependency and the role of S6K2 in supporting this addiction. Without wishing to be bound by any theory, it is believed that harnessing this S6K2-dependent addiction could be exploited as a potential strategy to combat MAPKi-resistant NRAS-mutant melanoma, a highly aggressive tumor type with limited treatment options.

### IV. METHODS

a. Cell Culture

Melanoma cell lines and 293T cells were grown in RPMI1640 supplemented with 5% and 10% FBS respectively. FBS was purchased from Tissue Culture Biologicals (#101; Lot #170708, 105094, 101258, 101929) or Clontech (#631107; Lot #A301117007).

b. 3D melanoma Spheroids.

Melanoma spheroids were generated and stained for live/dead cells as previously described (Echevarria-Vargas et al., 2018) with the following modifications: spheroids were embedded into a collagen mixture (10% RPMI, 1.5 mg/ml collagen, 10% FBS, and 7.5% NaHCO₃) and treated with DMSO or 50 μM Fenofibrate+7.5 μM DHA.

c. Source of Compounds and Chemicals

Cayman chemical: BVD-523 (#18298), Docosahexaenoic Acid (DHA; Cayman Chemical #90310 & MedChemExpress #HY-B2167/CS-6261), Fenofibrate (#10005368), GSH (#10007461), GSK2126458 (#17377), Kira6 (#19151), MEK-162 (#16996), SCH772984 (#19166), zVAD (zVAD(OMe)-FMK, #14463). Selleckchem: Ferrostatin-1 (#S7243), LY2584702 (#S7704), Trametinib (#S2673). Sigma-Aldrich: BrdU (5-Bromo-2′-deoxyuridine, #B5002), G418 (Sigma-Aldrich #G418-RO), PEG 300 (#202371), Propidium iodide (#P4864), RNase A (#R4642), Soybean oil (#S7381), TES (#T1375), Tween-80 (#P1754). BODIPY™ 581/591 C11 (Thermo Fisher Scientific, #D3861, Doxycycline HCl (Research Products International Crop. #D43020), MTT (BIOSYNTH #T-3450), PSVue 643 (Molecular Targeting Technologies P-1006), Puromycin (Gemini Bio Products, #400-128P).

d. Animal Studies.

All animal studies were approved by the Institutional Animal Care and Use Committee (IACUC) at the Wistar Institute and performed in accordance with institutional guidelines. Mice were housed in groups of five/cage in an Association for the Assessment and Accreditation of Laboratory Animal Care (AAALAC) certified facility. NOD/LtScidIL2Rγ-null (NSG) mice (4 to 8-week-old, ~25-30 g) were procured from The Wistar Institute. C57Bl/6 mice (4-week-old, ~25 g) were obtained from Charles River.

For in vivo xenograft studies, 1×10⁶ cells were resuspended in 1:1 RPMI1640/Matrigel (Matrigel Matrix, Corning #354230) and implanted subcutaneously into the flanks of male NSG mice. Patient-derived xenografts were minced, mixed with Matrigel and implanted into male NSG mice.

For the syngeneic mouse model, Nras-mutant tumors (WHN89) were derived from Tyr-CRE-ER^{T2} p16^{L/L} LSL-Nras^{Q61R/Q61R} (TpN^{61R/61R}) mouse model(44). TpN^{61R/61R} tumors were minced and implanted into male C57Bl/6 mice.

Tumor volume was measured using digital calipers and calculated by length×width²/2. Mice in all treatment groups were euthanized when the vehicle-treated group reached the humane endpoint (tumor volume 1500-2000 mm³).

PD0325901 treatment. Patient-derived xenografts were minced and subcutaneously injected into 8-week-old male NSG mice. When tumors reached 100-150 mm³, mice were randomly assigned into experimental groups. PD0325901 was prepared in 0.2% Tween 80+0.5% hydroxypropyl-methyl cellulose+3% DMSO in ddH2O and administered by oral gavage (1.5 or 5 mg/kg/day).

Fenofibrate combination. M93-047 cells were subcutaneously injected into 8-week-old male NSG mice. One week after tumor cell inoculation (tumor size ~40 mm³), mice were randomly assigned into five experimental groups: vehicle, fenofibrate, or fenofibrate+DHA. To study the effect of therapy in established tumors, M93-047 cells or minced patient-derived xenografts were subcutaneously injected into 4-week-old male NSG mice. When tumors reached 100-200 mm³, mice were randomly assigned into experimental groups. Syngeneic TpN^{61R/61R} tumor cells (WHN89) were subcutaneously injected into 4-week-old male C57Bl/6 mice. When tumors reached ~85 mm³, mice were randomly assigned into experimental groups and treated with vehicle, fenofibrate, DHA, or fenofibrate+DHA. Fenofibrate was prepared in 4% DMSO+40% PEG300+2% Tween 80 in ddH2O and administered by oral gavage (200 mg/kg/day). DHA was administered by oral gavage (300 mg/kg/day) in soybean oil. Mice bearing patient-derived xenografts were given control chow, or DHA (0.15% w/w) plus FNB (0.15% w/w) laced chow (BioServ).

e. Antibodies.

Primary antibodies: Actin (Sigma-Aldrich #A5441 1:10000), COX-2 (PTGS2) (Santa Cruz Biotechnology, Inc. #sc-19999 1:200. FLAG (Sigma-Aldrich #F4049, 1 μg/mL/1:1000), HA (Sigma-Aldrich #H9658, flow cytometry 1:2000), 4-HNE (R&D Systems #MAB3249, flow cytometry 1:100, IF 10 μg/mL/1:100, IHC 20 μg/mL/1:50), NCoR1 (Bethyl #A301-i 46A, WB 1:1000; Sigma-Aldrich #PLA0170; Bethyl #A301-145A, PLA 10 mg/mL), 8-OHdG (Santa Cruz Biotechnology, Inc. #sc-66036, flow cytometry 1:100, IF 1:100, IHC 1:200), PPARα (Santa Cruz Biotechnology, Inc. #sc-398394, WB 1:500, PLA 1:100), S6K2 (Abcam #ab76960, WB 1:500; Abnova #H00006199-B02P, PLA 2.5 μg/mL), pIRE1W (Novus #2323, 1:2000). All other antibodies were purchased from Cell Signaling Technology. Dilution 1:1000, unless otherwise specified. pAkt (T308; #4056), BrdU (#5292, flow cytometry 1:200), pERK (T202/Y204; #4370), pMEK (S217/221; #9121), pRSK (T359/5363; #9344), S6 (#2317), pS6 (S235/236; #4858), pS6 (S240/244; #5364, WB 1:500, flow cytometry 1:100), S6K1 (#2708), pS6K1 (T389; #9234), S6K2 (#14130 WB 1:1000, PLA 1:50), XBP1s (#12782), BIP (#3177 WB), p-eIF2α (#3597 WB), eIF2α (#2103), ATF4 (#11815), CHOP (#2895, 1:500), PERK (#3192).

Secondary antibodies: WB: 1:10000. IRDye 680 Conjugated Goat (polyclonal) Anti-Rabbit IgG (H+L) (LI-COR #926-32211). IRDye 800CW Conjugated Goat (polyclonal) Anti-Mouse IgG (H+L) (LI-COR #926-32220). Flow cytometry/IF secondary antibodies were purchased from Life Technologies; 1:500. Alexa Fluor 488 goat anti-rabbit IgG (H+L) (#A11008). Alexa Fluor 594 goat anti-rabbit IgG (H+L) (#A11012). Alexa Fluor 647 goat anti-rabbit IgG (H+L) (#A21244). Alexa Fluor 488 goat anti-mouse IgG (H+L) (#A11029). Alexa Fluor 594 goat anti-rabbit IgG (H+L) (#A11032).

f. Plasmids and Lentivirus.

Packaging plasmids pS-PAX2 and pMD2.G (a gift from Didier Trono (Addgene plasmid #12260, #12259)) were used to transfect 293T cells and produce lentiviral particles. Melanoma cells were transduced with lentivirus and selected with the appropriate antibiotics as previously described (Reyes-Uribe et al., 2018).

Plasmids: HA-tagged S6K1^{T389EΔCT} in pSLIK inducible vector was a gift from Kevin James (G418 selection, 2 mg/mL; Addgene #58516). Protein expression was induced with doxycycline (0.25 μg/mL). Flag-tagged S6K1^{T389S} in pLJM vector was a gift from David Sabatini (puromycin selection, 2 μg/mL; Addgene #48800). HA-tagged S6K2^{T388E}(Addgene #17731) was subcloned into pLUT-poly inducible vector (puromycin selection, 2 μg/mL; gift from Dr. Meenhard Herlyn, The Wistar Institute). S6K1 and S6K2 short hairpin RNAs in pLKO.1 vector. S6K1 shRNA (sh1: TRCN0000003158, sh4: TRNC0000003161, sh5: TRNC0000003162). S6K2 shRNA (sh1: TRNC0000000729, sh4: TRNC00000010540, sh5: TRCN0000010541, sh6: TRCN0000199513, sh7: TRCN 0000432273).

g. Immunoblotting.

Cells were lysed with RIPA buffer (50 mM Tris-HCl, pH 7.4, 150 mM NaCl, 1% NP-40, 0.5% sodium deoxycholate, 1 mM EDTA, 2.5 mM sodium pyrophosphate, 0.05% or 0.1% SDS) supplemented with sodium vanadate (0.2 mM) and protease inhibitor cocktail (Sigma-Aldrich #11697498001). Immunoblotting was performed as previously described (Echevarria-Vargas et al., 2018). Immunoblots were imaged with an Odyssey Infrared Imaging System (LI-COR Biosciences).

h. Immunostaining

Flow cytometry. Cells were fixed in 90% ice cold EtOH/PBS and stored at −20° C. Cells were washed twice with PBS followed by 1 h incubation at RT with incubation buffer (1% BSA/PBS) containing primary antibodies (1:500 pS6 S240/244 Cell Signaling Technology #5364; 1:1000 HA Sigma-Aldrich #H9658). Samples were washed once (1×PBS), incubated for 30 min at RT with incubation buffer containing secondary antibodies diluted to 1:500 (goat anti-mouse Alexa Fluor 488 ThermoFisher #A11029; goat anti-rabbit Alexa Fluor 647 #A21244), washed (1×PBS) and resuspended in incubation buffer for analysis by flow cytometry (BD LSRII 14-color flow cytometer analyser; Alexa Fluor 488 and APC filter sets). Data analyses were performed using FlowJo software to calculate pS6 positivity. For 4-HNE staining, cells were fixed with 4% paraformaldehyde for 15 min, washed with PBS and stored at 4° C. Before immunostaining, cells were permeabilized with 0.5% Triton X-100/PBS for 10 min.

Immunohistochemistry. Tumors were fixed with 10% buffered formalin (MedSupply Partners #8BUFF-FORM 10%), embedded in paraffin and cut into 4 mm consecutive sections. Deparaffinized sections were steamed in citrate buffer (pH 6) for 20 min or incubated in pressure cooker with DAKO EDTA (pH9, Agilent Dako #S2367) for 20 min to retrieve antigens. Samples were treated with 3% H₂O₂ solution for 10 min and then Triton X-100 (0.3% in PBS) for 15 min at RT. Samples were blocked with 5% horse serum in PBS for 1 h at RT, incubated with primary antibodies (4-HNE 1:50, 8-OHdG 1:200 in 4% BSA/PBS) overnight at 4° C., and incubated with secondary antibodies for 30 min at RT. All wash steps were performed with 0.5% PBST. Antigens were detected using diaminobenzidine (DAB). Slides were counterstained with hematoxylin. IHC images were acquired using a Nikon 80i upright microscope.

i. Proximity Ligation Assay

Cells were grown on chamber slides (Lab-Tek II #154534), fixed with 4% paraformaldehyde for 10 min and washed three times with PBS. Proximity ligation assay was performed using Duolink In Situ Red Starter Kit Mouse/Rabbit (Sigma-Aldrich #DU092101) per the manufacturer's instructions. Slides were mounted and analyzed on a Leica TCS SP5 II scanning spectral confocal microscope.

j. qRT-PCR

Cell pellets were homogenized using QIAshredder (QIagen #79656) and RNA extracted with PureLink RNA mini kit (Thermo Fisher Scientific, #12183018A). DNA was removed by on-column PureLink DNase treatment (Thermo Fisher Scientific, #12185010). First-strand cDNA was synthesized using Maxima cDNA kit (Thermo Scientific™ #K1642). Real time qPCR was performed using SYBR green PCR Master Mix (Applied Biosystems #4385612). Tumor samples for qRT-PCR were preserved in RNAlater solution (Invitrogen #AM7020).

k. Cell Viability Assays

MTT assay. Cells were seeded in 96-well plates (2500 cells/well) and treated with drugs by adding 2× compounds (prepared in fresh growth medium) to the cells. After 72 h, cell viability was determined by standard MTT Assays. Absorbance was determined at 490 nm using a BioTek plate reader.

CellTiter-Glo assay. Cells were seeded in 384-well plates (500 cells/well) and treated with drugs using the Janus MDT Nanohead. Viability was measured after 72 h using CellTiter-Glo (Promega). Data were normalized where 100% viability equals luminescence of DMSO-treated cells, and 0% viability equals luminescence of 1 μM bortezomib-treated cells.

l. Cell Proliferation Assay

Colorimetric detection. BrdU incorporation was performed per the manufacturer's instructions (BrdU Cell Proliferation Assay Kit; Cell Signaling Technology #6813). Briefly, cells were seeded in 96-well plates (2500 cells/well) one day before drug treatment. Cells were treated with drugs for 72 h, followed by BrdU labelling for 16 h and 30 min incubation in fixing/denaturing solution at RT. Cells were then incubated with primary antibody for 1 h at RT, washed three times followed by incubation with HRP-conjugated antibody solution at RT for 30 min. Colorimetric reactions for BrdU incorporation were started by incubation with TMB substrate (10 min at RT) and terminated by adding STOP solution; signal was determined by absorbance at 450 nm.

m. Cell Death Assay.

Floating and attached cells were harvested, washed and stained with PSVue 643/PI or Annexin V-FITC/PI. Annexin V staining was performed as described previously (45), with 15 min Annexin V incubation. For PSVue/PI staining, cells were incubated in 3 μM PSVue 643/TES for 10 min at RT, washed in TES buffer and resuspended in PI (5 μg/mL)/TES for 15 min at RT. Cell death (Annexin V⁺/PSVue⁺ or PI⁺) was quantified by FACS (LSRII 18 color analyzer, BD Biosciences) and analyzed using FlowJo software v.10.7.2.

n. Measurement of ROS Production.

Cells were trypsinized, washed with PBS and incubated with H2DCFDA [2′,7′-dichlorodihydrofluorescein diacetate, Thermo Fisher Scientific](10 μM in PBS) for 25 min at 37° C. Cells were then washed once with PBS to remove the dye and incubated in phenol red-free growth medium for 10 min at 37° C. Following incubation, cells were placed on ice, protected from light and immediately analyzed by flow cytometry.

o. Lipid Peroxidation Measurement.

Cells were harvested and incubated with Bodipy 581/591 C11 (5 μM in growth medium; Thermo Fisher Scientific) for 30 min at 37° C. Cells were washed 5 times with PBS and then analyzed by flow cytometry.

p. Proteomics

Sample preparation and LC-MS/MS analysis. M93-047 cells were transduced with vector control, S6K1 shRNA (sh1/4 or sh5) or S6K2 shRNA (sh1 or sh4) and lysed four days post-transduction with SDS buffer (50 mM Tris-HCl pH 7.5, 150 mM NaCl, 1% SDS and 1 mM EDTA) supplemented with protease inhibitors (150 mM PMSF, 1 mg/mL Pepstatin A and 1 mg/mL Leupeptin). In-gel trypsin digestion was performed using 15 mg (Beer et al., 2011).

LC-MS/MS of peptides was performed using a Waters nanoACQUITY UPLC in-line with a Thermo Q Exactive Plus mass spectrometer. For each sample, 1 mg of tryptic digest was loaded onto a 180 mm×2 cm nanoACQUITY UPLC Symmetry C18 trap column with 5 mm particle size (Waters #186006527) followed by analytical separation on a 1.7 mm×2.5 cm ACQUITY UPLC Peptide BEH C18 column with 1.7 μm particle size (Waters #186003546). Samples were analyzed in data-dependent mode using a 245 min LC gradient with water (solvent A) and acetonitrile (solvent B) containing 0.1% formic acid: 5-30% B over 225 min, 30-80% B over 5 min, 80% B hold for 10 min and return to initial conditions for 5 min. Full MS spectra were recorded at a resolution of 70,000 using a 400-2000 m/z scan range. Data-dependent MS/MS was performed on the top 20 most abundant ions selected with an isolation width of 1.5 m/z and were recorded at a resolution of 17,500.

Data analysis. RAW files were processed using MaxQuant 1.5.1.2 in a single run (Cox et al., 2008). Database searches were performed against the UniProt human sequence database (Jun. 29, 2017; 159,819 sequences) and an in-house database of common laboratory contaminants (Jul. 28, 2014; 3,671 sequences) with Trypsin/P specificity, a maximum of 2 missed cleavages and a minimum peptide length of 7 residues. Precursor and fragment mass tolerances were set to 4.5 ppm and 20 ppm, respectively. Protein N-terminal acetylation and methionine oxidation were set as variable modifications. Cysteine carbamidomethylation was set as a fixed modification. A maximum of 5 modifications were allowed per peptide. Peptide and protein false discovery rates were both set to 1%. Match between runs was enabled with a 0.7 min match time window and 20 min alignment time window. Protein quantification was based on unique peptides only. Label-free protein quantitation (LFQ) to normalize between samples was performed with a minimum peptide ratio of 1. Statistical analysis was performed using Perseus 1.5.0.31 (Tyanova et al., 2016). Protein groups identified by a single unique peptide or corresponding to contaminants were removed. LFQ intensities were log 2 transformed to reduce outlier effects, and missing values were imputed from a downshifted normal distribution. Statistical significance between conditions after pooling data from the two different shRNAs for the same S6K was defined as Student's t-test p-value less than 0.05 and absolute fold-change greater than 2 based on log 2-transformed LFQ intensities. Where indicated, correction for multiple testing was performed by permutation-based false discovery rate (FDR). Canonical pathway analysis was performed using QIAGEN's Ingenuity® Pathway Analysis software (IPA®, QIAGEN Redwood City, www.qiagen.com/ingenuity) with all identified proteins as the reference dataset.

q. Lipidomics

Sample preparation and LC-MS/MS analysis. M93-047 cells were transduced with vector control, S6K1 shRNA or S6K2 shRNA. Four days post transduction, lipids were extracted from cells grown in 10 cm plates using a modified Folch method (Folch et al., 1957). Briefly, media was removed from the plates and the cells were washed twice with PBS. Cells were quenched with ice-cold MeOH and transferred to a glass tube. Splash (Avanti Polar Lipids #330707) internal standard was added to each sample. Ice-cold 0.88% NaCl in water and CHCl₃ were added to a final ratio of 2:1:1 CHCl₃:MeOH:0.88% NaCl. Extracts were then sonicated and centrifuged. The lower organic phase was transferred to a new glass tube. The upper aqueous phase was re-extracted with synthetic organic phase, and the re-extracted organic phase was pooled with the initial extract. The pooled organic phases were dried under nitrogen, and lipids were reconstituted in 1:8:1 CHCl₃:MeOH:H₂O. Global lipid extracts from triplicate plates were analyzed by reversed-phase LC-MS/MS on a Thermo Q Exactive HF-X in both positive and negative polarities. For each polarity, 1% (2 μL) of the resuspended lipidome was loaded onto a 150 μm×2.1 cm Accucore C30 column with a 2.6 μm particle size (Thermo Fisher Scientific #27826-152130). The lipidome was analyzed using data-dependent acquisition on a 40-minute gradient with 1:1 acetonitrile:water with 5 mM ammonium formate and 0.1% formic acid (solvent A) and 88:10:2 isopropanol:acetonitrile:water with 5 mM ammonium formate and 0.1% formic acid (solvent B). The gradient is as follows: 0-60% B over 10 minutes, 60-85% B over 10 minutes, 85-100% over 10 minutes and holding for 5 minutes and re-equilibrating to initial conditions over 5 minutes. Full MS spectra were recorded at a resolution of 120,000 using a 300-1,200 m/z scan range in positive mode and 250-1,200 m/z scan range in negative mode. Data-dependent MS/MS scans were recorded at a resolution of 15,000 for the top 20 most abundant ions isolated with a 0.4 m/z isolation width. Stepped collision energy (NCE) of 20/30 was used in positive mode and 20/30/40 was used in negative mode.

Data analysis. Quantitation for global lipids was performed using Lipid Search 4.1 (Thermo Fisher Scientific). Briefly, raw files were processed using Lipid Search software in a single batch. Lipids were identified based on mass, retention time and predicted fragmentation patterns. All identified lipids are aligned and quantified with a 5 ppm mass tolerance and a retention time window of 0.2 minutes. Manual filtering included removing low scoring lipid identifications and unlikely adducts based on standards. To quantify lipid distribution, the abundance (peak area) of each lipid species in a sample was normalized to total lipid peak area in that sample. Statistical significance was defined as a Student's t-test p-value less than 0.05.

r. RNA Seq

Sample preparation. Cells were lysed in Tri-Reagent (Sigma-Aldrich, #T9424) and RNA extracted using the Direct-zol MiniPrep Kit (Zymo Research, #R2050) with on-column DNAse I treatment. Total RNA (200 ng) was used to prepare 3′ QuantSeq mRNASeq libraries (Lexogen, #KO152x96) as per protocol. Fourteen PCR cycles were used for amplification during the library preparation. Libraries were checked for quality using the TapeStation High Sensitivity D5000 ScreenTape (Agilent Technologies, #5067-5592). Quantitation of library was done using qPCR (Roche #KK4835). Sequencing was done on the NextSeq 500 (Illumina) per manufacturer's instructions using a 75 cycle High Output sequencing kit. A final concentration of 1.9 μM was loaded onto the flowcell.

Data analysis. RNA-seq data was aligned using bowtie2 (Langmead & Salzberg, 2012) against hg19 version of the human genome and RSEM v1.2.12 software (Li & Dewey, 2011) was used to estimate raw read counts and RPKM using Ensemble transcriptome. DESeq2 (Love et al., 2014) was used to estimate significance of differential expression between each shRNA and control condition. Genes with expression changes passing FDR<5% threshold were considered significant. Only genes that overlapped between the two shRNAs for S6K1 (sh1/4 and sh5) or S6K2 (sh1 and sh4) were analyzed. Heatmaps were normalized by DESeq2 log 2 scaled counts relative to average across all samples. Gene set enrichment analysis was done using QIAGEN's Ingenuity® Pathway Analysis software and significance of enrichment for Upstream Regulators was defined at p-value<0.05.

s. Reverse Phase Protein Array

Cells were lysed with 1% Triton X-100, 50 mM HEPES, pH 7.4, 150 mM NaCl, 1.5 mM MgCl₂, 1 mM EGTA, 10% glycerol, protease and phosphatase cocktail (Sigma-Aldrich #11873580001 and 04906837001). Lysates were denatured in sample buffer (10% glycerol, 2% SDS, 0.06 M Tris-HCl, pH 6.8, 1/40th volume of 2-ME) for 5 min, 95° C. RPPA was performed by the MD Anderson Center RPPA core facility as previously described (Tibes et al., 2006).

t. TCGA Data, S6K1 and S6K2 Correlation and Survival.

To determine the relationship between S6K2 and S6K1 expression in human melanoma patient tumors, log 2-transformed mRNA expression values for S6K1 and S6K2 were extracted from the TCGA Skin Cutaneous Melanoma (SKCM) dataset (n=443) via cBioPortal (accessed 5 Feb. 2022).

Kaplan-Meier survival curves for high vs. low S6K1 or S6K2 expression in melanoma patients (n=470) were obtained using the TCGA skin cutaneous melanoma dataset. The analysis was performed using R₂: Genomics Analysis and Visualization Platform (http://r2.ame.nl) to stratify by first (low, n=115) vs. last (high, n=115) quartiles. P values correspond to log-rank tests comparing the two Kaplan-Meier curves.

u. Tumor Growth Rate Analysis.

For the analysis of longitudinal data from in vivo tumor growth experiments, linear mixed effects models were applied to compare tumor growth rates between treatment groups. For multiple comparisons, FDR adjusted P-value <0.05 was considered significant.

v. Quantification and Statistical Analysis

Data were analyzed and plotted using GraphPad Prism or Microsoft Excel. Data are presented as mean±SD or median with interquartile range as indicated in each figure legend; replicate numbers (n) are indicated in the figure legends. In graphs showing mean of multiple independent experiments, values of technical replicates were first averaged to represent each experiment, and ‘n’ denotes how many independent experiments were performed. In graphs showing one representative experiment, ‘n’ denotes number of biological replicates. Statistical differences between two experimental groups were calculated using the unpaired two-tailed Student's t-test, unless otherwise specified.

### REFERENCES

The following references, to the extent that they provide exemplary procedural or other details supplementary to those set forth herein, are specifically incorporated herein by reference:
Alman za et al., FEBS J 286:241-278 (2019).
Beer et al., J Proteome Res 10:1126-1138 (2011).
Burd et al., Cancer Discov 4:1418-1429 (2014).
Cerezo et al., Cancer Cell 29:805-819 (2016).
Corcoran et al., Sci Transl Med 5:196ra198 (2013).
Cox & Mann, Nat Biotechnol 26:1367-1372 (2008).
Doll et al., Nat Chem Biol 13:91-98 (2017).
Echevarria-Vargas et al., EMBO Mol Med 10, (2018).
Folch et al., J Biol Chem 226:497-509 (1957).
Friedmann Angeli et al., Nat Cell Biol 16:1180-1191 (2014).
Gerstenecker et al., Cancers (Basel) 13, (2021).
Green, Cell 177:1094-1107 (2019).
Han & Kaufman, J Lipid Res 57:1329-1338 (2016).
Handbook of Pharmaceutical Salts: Properties, and Use, Stahl and Wermuth Eds., Verlag Helvetica Chimica Acta, 2002.
Hangauer et al., Nature 551:247-250 (2017).
Hodi et al., J. Clin. Oncol. 26(12): 2046-2051, 2008.
Jiang et al., Clin. Cancer Res. 14(23): 7726-7732, 2008.
Kansikan et al., Chem. Rev., 118(3):989-1068, 2018.
Karlsson et al., PLoS One 10:e0145013 (2015).
Kersten, Mol Metab 3:354-371 (2014).
Kim et al., Hepatology 55:1727-1737 (2012).
Langle-Rouault et al., J Virol., 72(7): 6181-6185, 1998.
Langmead & Salzberg, Nat Methods 9:357-359 (2012).
Lee et al., Proc Natl Acad Sci USA 117:32433-32442 (2020).
Levitskaya et al., Proc Natl Acad Sci USA., 94(23):12616-12621, 1997.
Li & Dewey, BMC Bioinformatics 12:323 (2011).
Lian et al., J Cancer 9:1527-1537 (2018).
Liu et al., Cell Rep Med 4:101206 (2023).
Love et al., Genome Biol 15:550 (2014).
Lu et al., J. Clin. Invest., 121(10):4015-29, 2011.
Lutzky et al., Pigment Cell Melanoma Res. 21 (4):492-493, 2008.
Magtanong et al., Cell Death Differ 23:1099-1109 (2016).
Pende et al., Mol Cell Biol 24:3112-3124 (2004).
Rakhshandehroo et al., PPAR Res 2010, (2010).
Reagan-Shaw et al., FASEB J., 22(3):659-661, 2008.
Reyes-Uribe et al., Oncogene 37:4058-4072 (2018).
Romano et al., Cancer Discov 8:556-567 (2018).
Rufo et al., Trends in Cancer 3:643-658 (2017).
Sengupta et al., J Invest Dermatol 133:1731-1741 (2013).
Shimada et al., Nat Chem Biol 12:497-503 (2016).
Sivachandran et al., PLoS Pathog., 4:10, 2008.
Szegezdi et al., EMBO Rep 7:880-885 (2006).
Teh et al., Cancer Discov 8:568-581 (2018).
Tibes et al., Mol Cancer Ther 5:2512-2521 (2006).
Tsoi et al., Cancer Cell 33:890-904 e895 (2018).
Tyanova et al., Nat Methods 13:731-740 (2016).
Villanueva et al., Cell Rep 4:1090-1099 (2013).
Viros er al., PLoS Med 5(6): 120, 2008.
Viswanathan et al., Nature 547:3-457 (2017).
Wang et al., Cell 173:1413-1425.e1414 (2018).
Yang et al., Cell 156:317-331 (2014).

What is claimed:

1. A compound of the formula: 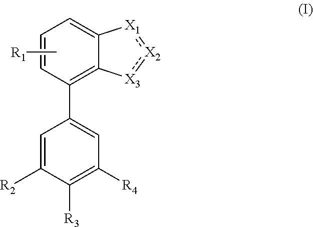
wherein:
R₁ is hydrogen, hydroxy, halo or amino;
R₂, R₃, and R₄ are each independently hydrogen, hydroxy, alkylamino_{(C≤8)}, substituted alkylamino_{(C≤8)}, dialkylamino_{(C≤8)}, substituted dialkylamino_{(C≤8)}, or
—NR_{b}C(X₄)(C(R_{c}R_{d}Rₑ))_{y}, wherein
R_{b} is hydrogen, alkyl_{(C≤12)}, or substituted alkyl_{(C≤12)};
R_{c}, R_{d} and Rₑ are each independently absent, halo, hydrogen, hydroxy or amino;
X₄ is O or NR_{f}, wherein R_{f} is hydrogen, alkyl_{(C≤8)}, or substituted alkyl_{(C≤8)};
y is 1, 2 or 3; or
—NR_{b}S(O)₂(C(R_{c}R_{d}Rₑ))_{y}, wherein
R_{b} is hydrogen, alkyl_{(C≤12)}, or substituted alkyl_{(C≤12)};
R_{c}, R_{d} and Rₑ are each independently absent, halo, hydrogen, hydroxy or amino;
y is 1, 2 or 3; or
—CNR_{b}C(X₄)(C(R_{c}R_{d}Rₑ))_{y}, wherein
R_{b} is hydrogen, alkyl_{(C≤12)}, or substituted alkyl_{(C≤12)};
R_{c}, R_{d} and Rₑ are each independently absent, halo, hydrogen, hydroxy or amino;
X₄ is O or NR_{f}, wherein R_{f} is hydrogen, alkyl_{(C≤8)}, or substituted alkyl_{(C≤8)};
y is 1, 2 or 3; or
A group of the formula: 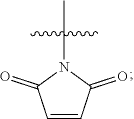 [SMILES: *N1C(=O)C=CC1=O]
provided that R₂, R₃, and R₄ are not each hydrogen; and
X₁, X₂, and X₃ are each independently O, S, C(O), CH, CH₂ or NR_{g}, wherein R_{g} is hydrogen, alkyl_{(C≤8)}, or substituted alkyl_{(C≤8)};
or a pharmaceutically acceptable salt thereof.

2. The compound of claim 1, further defined as: 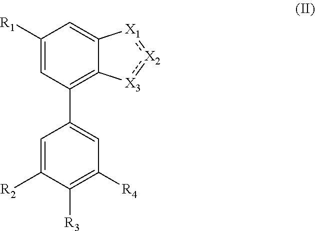
wherein:
R₁ is hydrogen or hydroxy;
R₂, R₃, and R₄ are each independently hydrogen, hydroxy, alkylamino_{(C≤8)}, substituted alkylamino_{(C≤8)}, dialkylamino_{(C≤8)}, substituted dialkylamino_{(C≤8)}, or
—NR_{b}C(X₄)(C(R_{c}R_{d}Rₑ))_{y}, wherein
R_{b} is hydrogen, alkyl_{(C≤12)}, or substituted alkyl_{(C≤12)};
R_{c}, R_{d} and Rₑ are each independently absent, halo, hydrogen, hydroxy or amino;
X₄ is O or NR_{f}, wherein R_{f} is hydrogen, alkyl_{(C≤8)}, or substituted alkyl_{(C≤8)};
y is 1, 2 or 3; or
—NR_{b}S(O)₂(C(R_{c}R_{d}Rₑ))_{y}, wherein
R_{b} is hydrogen, alkyl_{(C≤12)}, or substituted alkyl_{(C≤12)};
R_{c}, R_{d} and Rₑ are each independently absent, halo, hydrogen, hydroxy or amino;
y is 1, 2 or 3; or
—CNR_{b}C(X₄)(C(R_{c}R_{d}Rₑ))_{y}, wherein
R_{b} is hydrogen, alkyl_{(C≤12)}, or substituted alkyl_{(C≤12)};
R_{c}, R_{d} and Rₑ are each independently absent, halo, hydrogen, hydroxy or amino;
X₄ is O or NR_{f}, wherein R_{f} is hydrogen, alkyl_{(C≤8)}, or substituted alkyl_{(C≤8)};
y is 1, 2 or 3; or
a group of the formula: 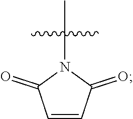 [SMILES: *N1C(=O)C=CC1=O]
provided that R₂, R₃, and R₄ are not each hydrogen; and
X₁, X₂, and X₃ are each independently O, S, C(O), CH or CH₂;
or a pharmaceutically acceptable salt thereof.

3. The compound of either claim 1 or claim 2, further defined as: 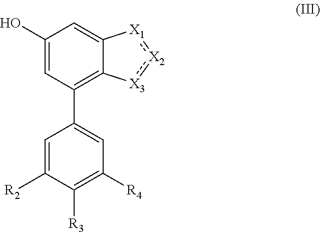
wherein:
R₂, R₃, and R₄ are each independently hydrogen, hydroxy, alkylamino_{(C≤8)}, substituted alkylamino_{(C≤8)}, dialkylamino_{(C≤8)}, substituted dialkylamino_{(C≤8)}, or
—NR_{b}C(X₄)(C(R_{c}R_{d}Rₑ))_{y}, wherein
R_{b} is hydrogen, alkyl_{(C≤12)}, or substituted alkyl_{(C≤12)};
R_{c}, R_{d} and Rₑ are each independently absent, halo, hydrogen, hydroxy or amino;
X₄ is O or NR_{f}, wherein R_{f} is hydrogen, alkyl_{(C≤8)}, or substituted alkyl_{(C≤8)};
y is 1, 2 or 3; or
—NR_{b}S(O)₂(C(R_{c}R_{d}Rₑ))_{y}, wherein
R_{b} is hydrogen, alkyl_{(C≤12)}, or substituted alkyl_{(C≤12)};
R_{c}, R_{d} and Rₑ are each independently absent, halo, hydrogen, hydroxy or amino;
y is 1, 2 or 3; or
—CNR_{b}C(X₄)(C(R_{c}R_{d}Rₑ))_{y}, wherein
R_{b} is hydrogen, alkyl_{(C≤12)}, or substituted alkyl_{(C≤12)};
R_{c}, R_{d} and Rₑ are each independently absent, halo, hydrogen, hydroxy or amino;
X₄ is O or NR_{f}, wherein R_{f} is hydrogen, alkyl_{(C≤8)}, or substituted alkyl_{(C≤8)};
y is 1, 2 or 3; or
A group of the formula: 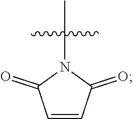 [SMILES: *N1C(=O)C=CC1=O]
provided that R₂, R₃, and R₄ are not each hydrogen; and
X₁, X₂, and X₃ are each independently O, S, C(O), CH or CH₂;
or a pharmaceutically acceptable salt thereof.

4. The compound according to any one of claims 1-3, further defined as: 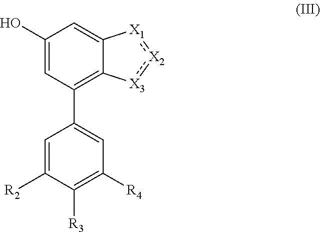
wherein:
R₂, R₃, and R₄ are each independently hydrogen, hydroxy, alkylamino_{(C≤8)}, substituted alkylamino_{(C≤8)}, dialkylamino_{(C≤8)}, substituted dialkylamino_{(C≤8)}, or
—NR_{b}C(X₄)(C(R_{c}R_{d}Rₑ))_{y}, wherein
R_{b} is hydrogen, alkyl_{(C≤12)}, or substituted alkyl_{(C≤12)};
R_{c}, R_{d} and Rₑ are each independently absent, halo, hydrogen, hydroxy or amino;
X₄ is O or NR_{f}, wherein R_{f} is hydrogen, alkyl_{(C≤8)}, or substituted alkyl_{(C≤8)};
y is 1, 2 or 3;
provided that R₂, R₃, and R₄ are not each hydrogen; and
X₁, X₂, and X₃ are each independently O, S, C(O), CH or CH₂;
or a pharmaceutically acceptable salt thereof.

5. The compound according to any one of claims 1-4, further defined as: 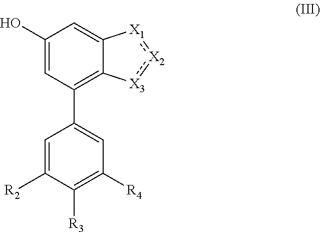
wherein:
R₂, R₃, and R₄ are each independently hydrogen, hydroxy, alkylamino_{(C≤8)}, substituted alkylamino_{(C≤8)}, dialkylamino_{(C≤8)}, substituted dialkylamino_{(C≤8)}, or
—NR_{b}S(O)₂(C(R_{c}R_{d}Rₑ))_{y}, wherein
R_{b} is hydrogen, alkyl_{(C≤12)}, or substituted alkyl_{(C≤12)};
R_{c}, R_{d} and Rₑ are each independently absent, halo, hydrogen, hydroxy or amino;
y is 1, 2 or 3;
provided that R₂, R₃, and R₄ are not each hydrogen; and
X₁, X₂, and X₃ are each independently O, S, C(O), CH or CH₂;
or a pharmaceutically acceptable salt thereof.

6. The compound according to any one of claims 1-5, further defined as: 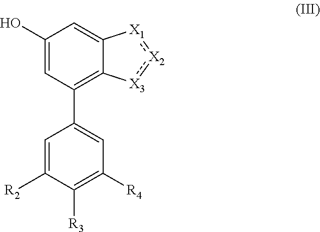
wherein:
R₂, R₃, and R₄ are each independently hydrogen, hydroxy, alkylamino_{(C≤8)}, substituted alkylamino_{(C≤8)}, dialkylamino_{(C≤8)}, substituted dialkylamino_{(C≤8)}, or
—NR_{b}C(O)(C(R_{c}R_{d}Rₑ))_{y}, wherein
R_{b} is hydrogen, alkyl_{(C≤12)}, or substituted alkyl_{(C≤12)};
R_{c}, R_{d} and Rₑ are each independently absent, halo, hydrogen, hydroxy or amino;
y is 1, 2 or 3;
provided that R₂, R₃, and R₄ are not each hydrogen; and
X₁, X₂, and X₃ are each independently O, S, C(O), CH or CH₂;
or a pharmaceutically acceptable salt thereof.

7. The compound of either claim 1 or claim 2, wherein R₁ is hydroxy.

8. The compound according to any one of claims 1-7, wherein R₂ is hydrogen.

9. The compound according to any one of claims 1-4 and 7, wherein R₂ is —NR_{b}C(X₄)(C(R_{c}R_{d}Rₑ))_{y}.

10. The compound of claim 9, wherein X₄ is O.

11. The compound of claim 9, wherein R_{b} is hydrogen.

12. The compound of claim 9, wherein y is 1.

13. The compound of claim 9, wherein R_{c} and R_{d} are hydrogen.

14. The compound of claim 9, wherein Rₑ is halo.

15. The compound of claim 14, wherein Rₑ is chloro.

16. The compound according to any one of claims 1-15, wherein R₃ is hydrogen.

17. The compound according to any one of claims 1-4 and 7, wherein R₃ is —NR_{b}C(X₄)(C(R_{c}R_{d}Rₑ))_{y}.

18. The compound of claim 17, wherein X₄ is O.

19. The compound of claim 17, wherein R_{b} is hydrogen.

20. The compound of claim 17, wherein y is 1.

21. The compound of claim 17, wherein R_{c} and R_{d} are hydrogen.

22. The compound of claim 17, wherein Rₑ is halo.

23. The compound of claim 22, wherein Rₑ is chloro.

24. The compound according to any one of claims 1-4 and 7, wherein R₃—N(H)S(O)₂CH₂(Cl).

25. The compound according to any one of claims 1-24, wherein R₄ is hydrogen.

26. The compound according to any one of claims 1-25, wherein X₁ is S.

27. The compound according to any one of claims 1-25, wherein X₁ is O.

28. The compound according to any one of claims 1-25, wherein X₁ is NH.

29. The compound according to any one of claims 1-28, wherein X₂ is C(O).

30. The compound according to any one of claims 1-28, wherein X₂ is CH.

31. The compound according to any one of claims 1-30, wherein X₃ is O.

32. The compound according to any one of claims 1-30, wherein X₃ is CH.

33. The compound according to any one of claims 1-32, wherein the compound is further defined as: 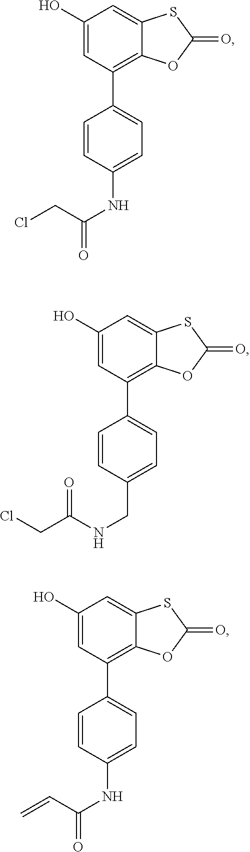 [SMILES: O=C(CCl)Nc1ccc(-c2cc(O)cc3sc(=O)oc23)cc1 ; O=C(CCl)NCc1ccc(-c2cc(O)cc3sc(=O)oc23)cc1 ; C=CC(=O)Nc1ccc(-c2cc(O)cc3sc(=O)oc23)cc1] 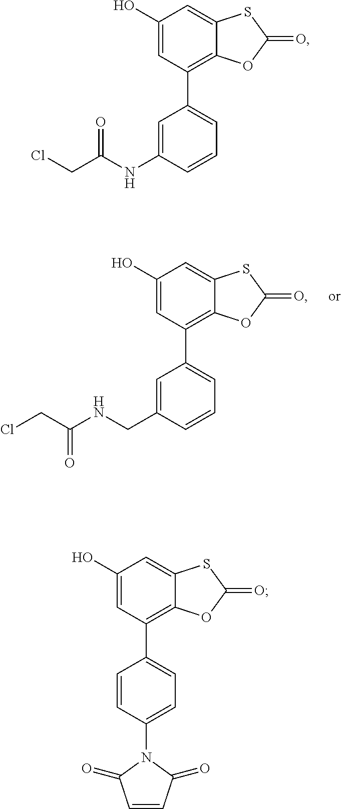 [SMILES: O=C(CCl)Nc1cccc(-c2cc(O)cc3sc(=O)oc23)c1 ; O=C(CCl)NCc1cccc(-c2cc(O)cc3sc(=O)oc23)c1 ; O=C1C=CC(=O)N1c1ccc(-c2cc(O)cc3sc(=O)oc23)cc1]
or a pharmaceutically acceptable salt thereof.

34. A pharmaceutical composition comprising:
(A) a compound according to any one of claims 1-33; and
(B) a pharmaceutically acceptable excipient.

35. The pharmaceutical composition of claim 34, wherein the pharmaceutical composition is formulated for administration: orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, parenterally, rectally, subconjunctival, subcutaneously, sublingually, topically, transbuccally, transdermally, vaginally, in cremes, in lipid compositions, via a catheter, via a lavage, via continuous infusion, via infusion, via inhalation, via injection, via local delivery, or via localized perfusion.

36. The pharmaceutical composition of claim 35, wherein the pharmaceutical composition is formulated for oral, intraarterial, or intravenous administration.

37. The pharmaceutical composition according to any one of claims 34-36, wherein the pharmaceutical composition is formulated as a unit dose.

38. A method of treating or preventing a disease or disorder in a patient in need thereof comprising administering to the patient a therapeutically effective amount of a compound or composition according to any one of claims 1-37.

39. The method of claim 38, wherein the disease or disorder is cancer.

40. The method of claim 39, wherein the cancer is a melanoma.

41. The method of claim 40, wherein the melanoma is acral lentiginous melanoma.

42. The method according to any one of claims 38-41, wherein the patient is of Asian descent.

43. The method according to any one of claims 38-41, wherein the patient is of sub-Saharan African descent.

44. The method according to any one of claims 38-43, wherein the patient is a mammal.

45. The method of claim 44, wherein the mammal is a human.

46. The method according to any one of claims 38-45, wherein the method further comprises a second therapy.

47. The method of claim 46, wherein the second therapy is a chemotherapeutic agent, immunotherapy, or a radiotherapy.

48. The method of claim 47, wherein the second therapy is an inhibitor of histone demethylase (KDM).

49. The method of claim 48, wherein the second therapy is an inhibitory nucleic acid for a histone demethylase.

50. The method according to any one of claims 38-49, wherein the disease or disorder is a cancer with a mutation in the RAS gene.

51. The method of claim 50, wherein the cancer with a mutation in the RAS gene is a melanoma.

51.1. The method of claim 50 or claim 51, wherein the RAS gene is NRAS.

51.2. The method according to any one of claims 38-51.1, wherein the disease or disorder is resistant to one or more therapeutic agents.

51.3. The method of claim 51.2, wherein the disease or disorder is cancer is resistant to one or more therapeutic agents.

51.4. The method of either claim 51.2 or claim 51.3, wherein the therapeutic agent is a therapeutic agent that inhibits MAPK.

51.5. The method according to any one of claims 52-64, wherein the inhibition of the S6K2 results in the treatment of a melanoma that contains a mutation in NRAS and is resistant to one or more MAPK inhibitors.

51.6. The method of claim 51.5, wherein the inhibition of the S6K2 results in the treatment of a melanoma that contains a mutation in NRAS and is resistant to all MAPK inhibitors.

52. A method of inhibiting the activity of ribosomal protein S6 kinase B2 (S6K2) in a patient comprising administering to the patient a therapeutically effective amount of a compound or a pharmaceutical composition according to any one of claims 1-37.

53. The method of claim 52, wherein the compound or composition shows a 50% decrease in the inhibition for ribosomal protein S6 kinase B1 (S6K1) relative to the inhibition of S6K2.

54. The method of claim 53, wherein the inhibition of S6K1 is less than 25% of the inhibition of S6K2.

55. The method of either claim 53 or claim 54, wherein the inhibition of S6K1 is less than 5% of the inhibition of S6K2.

56. The method according to any one of claims 52-55, wherein the inhibition of S6K2 is sufficient to treat a disease or disorder.

57. The method of claim 56, wherein the disease or disorder is cancer.

58. The method of claim 57, wherein the cancer is skin cancer.

59. The method of claim 58, wherein the skin cancer is a melanoma.

60. The method according to any one of claims 57-59, wherein the cancer contains one or more mutations in the RAS family of genes.

61. The method of claim 60, wherein the mutation is a mutation in NRAS.

62. The method according to any one of claims 56-61, wherein the disease or disorder is resistant to one or more therapeutic agents.

63. The method of claim 62, wherein the cancer is resistant to one or more therapeutic agents.

64. The method of either claim 62 or claim 63, wherein the therapeutic agent is a therapeutic agent that inhibits MAPK.

65. The method according to any one of claims 52-64, wherein the inhibition of the S6K2 results in the treatment of a melanoma that contains a mutation in NRAS and is resistant to one or more MAPK inhibitors.

66. The method of claim 65, wherein the inhibition of the S6K2 results in the treatment of a melanoma that contains a mutation in NRAS and is resistant to all MAPK inhibitors.

67. A method of depleting a cell of ribosomal protein S6 kinase B2 (S6K2) in a patient comprising administering to the patient a therapeutically effective amount of a compound or a pharmaceutical composition according to any one of claims 1-37.

68. The method of claim 67, wherein the compound or composition shows a 50% decrease in the depletion of ribosomal protein S6 kinase B1 (S6K1) relative to the depletion of S6K2.

69. The method of claim 68, wherein the depletion of S6K1 is less than 25% of the depletion of S6K2.

70. The method of either claim 68 or claim 69, wherein the depletion of S6K1 is less than 5% of the depletion of S6K2.

71. The method according to any one of claims 67-70, wherein the depletion of S6K2 is sufficient to treat a disease or disorder.

72. The method of claim 71, wherein the disease or disorder is cancer.

73. The method of claim 72, wherein the cancer is skin cancer.

74. The method of claim 73, wherein the skin cancer is a melanoma.

75. The method according to any one of claims 71-74, wherein the cancer contains one or more mutations in the RAS family of genes.

76. The method of claim 75, wherein the mutation is a mutation in NRAS.

77. The method according to any one of claims 67-76, wherein the disease or disorder is resistant to one or more therapeutic agents.

78. The method of claim 77, wherein the cancer is resistant to one or more therapeutic agents.

79. The method of either claim 77 or claim 78, wherein the therapeutic agent is a therapeutic agent that inhibits MAPK.

80. The method according to any one of claims 67-79, wherein the depletion of the S6K2 results in the treatment of a melanoma that contains a mutation in NRAS and is resistant to one or more MAPK inhibitors.

66. The method of claim 65, wherein the inhibition of the S6K2 results in the treatment of a melanoma that contains a mutation in NRAS and is resistant to all MAPK inhibitors.
